(19) United States
(12) Patent Application Publication
(10) Pub. No.: US 2005/0260272 A1
(43) Pub. Date: Nov. 24, 2005

(54) Method of forming microparticles that include a bisphosphonate and a polymer

(75) Inventors: Maria C. Figueiredo, Somerville, MA (US); Rajesh Kumar, Marlborough, MA (US); Maura J. Maloney, Hingham, MA (US); Kristin Prinn, Somerville, MA (US); David S. Scher, Hudson, MA (US); Gregory C. Troiano, Weymouth, MA (US); Thean Y. Yeoh, Foxboro, MA (US); Stephen E. Zale, Hopkinton, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Alkermes Controlled Therapeutics, Inc., Cambridge, MA (US)

(21) Appl. No.: 11/122,796

(22) Filed: May 5, 2005

Related U.S. Application Data
(60) Provisional application No. 60/568,467, filed on May 5, 2004.

(51) Int. Cl. A61K 9/14; A61K 9/50; B01J 13/02; B01J 13/04

(52) U.S. Cl. 424/489; 264/4.1

(57) ABSTRACT

Microparticles that include a bisphosphonate and a polymer are produced by a method that includes forming a water-in-oil emulsion by mixing an aqueous solution of the bisphosphonate with a combination of a biocompatible polymer and a polymer solvent. At least one aqueous liquid can be mixed with the water-in-oil emulsion to form a water-in-oil-in-water emulsion and to extract the polymer solvent from the polymer, thereby forming the microparticles. Methods of treating a patient in need of therapy include administering the microparticles described to the patient. In one embodiment, the microparticles are formulated for the sustained release of the bisphosphonate.

### RELATED APPLICATION

This application claims the benefit of U.S. Provisional Application No. 60/568,467, filed on May 5, 2004, the entire teachings of which are incorporated herein by reference.

### BACKGROUND OF THE INVENTION

Many illnesses or conditions require administration of a constant or sustained level of an active agent to provide the desired prophylactic, therapeutic, or diagnostic effect. This can be accomplished through a multiple dosing regimen or by employing a system that releases the active agent in a sustained fashion.

Attempts to sustain medication levels include the use of biodegradable compositions, such as biocompatible polymers having incorporated therein one or more active agents. The use of these biodegradable polymer/active agent compositions, for example, in the form of microparticles or microcarriers, can provide sustained release of active agents by utilizing the inherent biodegradability of the polymer. The ability to provide a sustained level of the active agent can result in improved patient compliance and therapeutic effects.

However, such biodegradable polymer/active agent compositions can exhibit high release of the active agent over the first twenty-four hours, often referred to as a “burst.” In some instances, this burst can result in an undesirable increase in levels of the active agent and minimal release of the active agent thereafter. In addition, due to the high concentrations of the active agent within and localized around these biodegradable polymer/active agent compositions in vivo, local irritation, inflamation and injection site swelling can result.

In view of the above, improved methods for the formation of microparticles and pharmaceutical compositions containing the microparticles are needed. For example, a need exists for methods of preparing biodegradable polymer/active agent compositions wherein the burst of agent can be reduced and/or wherein an improved release profile, e.g., a longer period of release, can be provided.

### SUMMARY OF THE INVENTION

The present invention relates to methods of forming microparticles that include a bisphosphonate and a polymer. The invention also includes microparticles produced by these methods and methods of treating a patient in need of therapy that include administering the microparticles described herein to the patient. In one embodiment, the microparticles are formulated for the sustained release of the bisphosphonate.

The method of forming microparticles that include a bisphosphonate and a polymer can include the step of forming a water-in-oil emulsion by mixing an aqueous solution of the bisphosphonate with a combination of a biocompatible polymer and a polymer solvent. At least one aqueous liquid can be mixed with the water-in-oil emulsion to form a water-in-oil-in-water emulsion and to extract the polymer solvent from the polymer, thereby forming the microparticles. In one aspect of the invention, the aqueous solution of the bisphosphonate consists essentially of water and the bisphosphonate.

In one embodiment, a method of forming microparticles includes forming a water-in-oil emulsion by mixing an aqueous solution of the bisphosphonate with a combination of a poly(lactide) or a poly(lactide-co-glycolide) polymer and a polymer solvent, wherein the molar ratio of the lactide component to the glycolide component in the polymer is at least about 65:35. Then, at least one aqueous liquid can be mixed with the water-in-oil emulsion to form a water-in-oil-in-water emulsion and to extract the polymer solvent from the polymer, thereby forming the microparticles.

In another embodiment, a method of forming microparticles includes forming a water-in-oil emulsion by mixing an aqueous solution of the bisphosphonate with a combination of a biocompatible polymer and a polymer solvent, wherein the concentration of the bisphosphonate in the aqueous solution is greater than the room temperature solubility limit of the bisphosphonate; and mixing at least one aqueous liquid with the water-in-oil emulsion to form a water-in-oil-in-water emulsion and to extract the polymer solvent from the polymer, thereby forming the microparticles.

Another method of forming microparticles that include a bisphosphonate and a polymer includes the step of preparing an aqueous mixture of the bisphosphonate and a surfactant. A water-in-oil emulsion can be formed by mixing the aqueous mixture of the bisphosphonate and a surfactant with a combination of a biocompatible polymer and a polymer solvent, a water-in-oil-in-water emulsion can be formed by mixing the water-in-oil emulsion with an aqueous liquid, and the polymer solvent can be removed from the polymer to form the microparticles.

The present invention also includes a method of forming microparticles that include a bisphosphonate and a polymer wherein a bisphosphonate suspension is formed in a combination consisting essentially of a biocompatible polymer and a polymer solvent; and at least one aqueous liquid is mixed with the bisphosphonate suspension to form a solid-in-oil-in-water emulsion and to extract the polymer solvent from the polymer, thereby forming the microparticles.

In one specific embodiment, the method of forming microparticles that include a bisphosphonate and a polymer includes the steps of forming a water-in-oil emulsion by mixing an aqueous solution of the bisphosphonate with a combination of a biocompatible polymer and a polymer solvent, wherein the concentration of the bisphosphonate in the aqueous solution is greater than the room temperature (e.g., about 21 to about 23° C.) solubility limit of the bisphosphonate; forming a water-in-oil-in-water emulsion by mixing an aqueous liquid with the water-in-oil emulsion; and extracting the polymer solvent from the polymer into another aqueous liquid, thereby forming the microparticles.

The present invention includes microparticles formed by the methods described herein and also pharmaceutical compositions that contain the microparticles, e.g., pharmaceutical compositions for the sustained release of a bisphosphonate. The present invention also relates to microparticles that are gamma-irradiated. In one embodiment, the microparticles are gamma-irradiated with about 15 to about 45 KiloGrays (kGy) of gamma radiation. For example, in one specific embodiment, the microparticles are gamma-irradiated with about 16 kGy of gamma radiation. In another specific embodiment, the microparticles are gamma-irradiated with about 26 kGy of gamma radiation.

The present invention also relates to microparticles consisting essentially of a biocompatible polymer and at least about 2 weight percent of risedronate or a salt thereof, e.g., at least about 3 weight percent of risedronate or a salt thereof. In one embodiment, the present invention includes microparticles that consist essentially of a bisphosphonate and a biocompatible polymer wherein the microparticles have an in vitro 24-hour cumulative bisphosphonate release of less than about 15 weight percent. The in vitro 24-hour bisphosphonate (e.g., risedronate) release can be in a phosphate buffered saline composition such as a phosphate buffered saline composition at 37° C. containing 0.02 weight percent polysorbate 20.

The invention described herein also includes microparticles consisting essentially of a bisphosphonate and a biocompatible polymer wherein the microparticles cause a reduced local site reaction in vivo upon parenteral administration to a patient as compared to a local site reaction caused by a parenteral administration to the patient of a bisphosphonate not formed into microparticles with a biocompatible polymer. In one embodiment, microparticles consisting essentially of a bisphosphonate and a biocompatible polymer cause a local site reaction in vivo upon parenteral administration to a patient that is substantially similar to a local site reaction caused by placebo microparticles that include the biocompatible polymer. In one aspect of the invention, microparticles consisting essentially of a bisphosphonate and a biocompatible polymer have clinically acceptable local tolerability in vivo upon administration to a patient. Additionally, methods for treating a patient that include administering the microparticles of the present invention are described herein.

In one embodiment, the present invention includes microparticles comprising a poly(d,l-lactide-co-gylcolide) polymer having about 75 mol % d,l-lactide, about 25 mol % glycolide, and a lauryl ester end group; and risedronate or a salt thereof; wherein the volume median diameter of the microparticles is about 20 to about 60 microns, for example, about 45 to about 55 microns, about 35 to about 45 microns, or about 25 to about 35 microns. In another embodiment, the microparticles comprise a poly(d,l-lactide-co-gylcolide) polymer having about 65 mol % d,l-lactide, about 35 mol % glycolide, and a lauryl ester end group; and risedronate or a salt thereof; wherein the volume median diameter of the microparticles is about 40 to about 60 microns, for example, about 45 to about 55 microns. In yet another embodiment, the microparticles comprise a poly(d,l-lactide) polymer having a methyl ester end group; and risedronate or a salt thereof; wherein the volume median diameter of the microparticles is about 40 to about 60 microns, for example, about 45 to about 55 microns. In some embodiments, the microparticles include risedronate, or a salt thereof, at a concentration of about 3 to about 6 percent by weight.

The methods described herein can provide for efficient, facile and cost effective formation of microparticles having desirable physical and chemical properties. For example, the microparticles prepared according to the methods described herein can exhibit a reduced initial release of a bisphosphonate, can provide a higher sustained level of the bisphosphonate, and/or can provide a longer duration of release of the bisphosphonate in vivo than generally is provided by known release systems.

The microparticles described herein can be administered to a patient by subcutaneous injection. In some embodiments, subcutaneous injection of microparticles can provide sustained levels of the active agent and can therefore result in improvements in patient compliance and/or therapeutic effects. For example, in some embodiments, subcutaneously administered microparticles can eliminate certain dosing requirements associated with an oral formulation such as, for example, reduced bioavailability associated with food effects, gastrointestinal adverse effects and/or intolerance, and recommendations for upright posture associated with gastrointestinal effects.

The microparticles of the present invention can be administered to a patient with a resulting improved local tolerability at the site of administration. Without being held to any particular theory, it is believed that improved local tolerability results, at least in part, from a reduced initial bisphosphonate release from the microparticles in vivo than generally occurs in known systems. In one embodiment, the microparticles described herein can reduce or substantially prevent adverse reaction to the bisphosphonate at an administration site that can otherwise occur using other means for bisphosphonate delivery. The microparticles described herein can be particularly suited for the delivery of bisphosphonates such as risedronate which can produce significant adverse effects at a site of administration when administered, for example, in a bulk state, e.g., as a solution.

In some embodiments, the microparticles formed using the methods described herein can be administered to patients using relatively small gauge needles. For example, in some embodiments, the microparticles can be effectively delivered to a patient using a 23 or 25 gauge needle. In other embodiments, the microparticles can be effectively delivered using even smaller needles. In other embodiments, the microparticles can be effectively delivered to a patient using a needle-free injection device.

In some embodiments, the microparticles described herein can provide higher sustained levels and/or longer durations of release of the bisphosphonate in vivo. For example, it is thought that since the microparticles can produce a reduced initial bisphosphonate release from the microparticles in vivo than is generally provided by known systems, more of the bisphosphonate can be available in the microparticles for later release and thus can provide higher levels of bisphosphonate and/or longer duration of release from the microparticles. The duration of release of a bisphosphonate from the present microparticles can be longer than the duration of release that can be provided by other known bisphosphonate compositions. For example, in some embodiments, a bisphosphonate can be released from the microparticles in vivo for up to about 3 months to about 6 months or more.

Without being held to any particular theory, it is believed that one factor in the microparticles providing higher sustained levels and/or longer durations of release of the bisphosphonate in vivo is the crystal form and/or size of the bisphosphonate. Further, it is believed that by practicing the methods described herein for producing microparticles, microparticles can be formed while controlling the bisphosphonate crystal form and/or size.

The microparticles of the present invention can include simple combinations of the bisphosphonate and a polymer, while having improved properties in vivo, as compared to known bisphosphonate compositions. For example, practice of the methods of the present invention can produce microparticles that consist essentially of a biocompatible polymer and a bisphosphonate while providing reduced initial release of a bisphosphonate, higher sustained levels of the bisphosphonate, and/or longer durations of release of the bisphosphonate in vivo. The microparticles described herein can provide improved bisphosphonate release characteristics in vivo without additional bisphosphonate release-modifying components.

The present methods can be used to form microparticles with a high bisphosphonate loading efficiency. For example, the present methods can be used to form microparticles with bisphosphonate loading efficiencies of about 60, 70, 80, or about 90 weight percent or more as compared to theoretical estimates of loading. The present microparticles can be formed having relatively large loadings of water soluble bisphosphonates, such as risedronate sodium, while using a process that includes the use of relatively large quantities of aqueous liquids.

Practice of the methods for forming microparticles described herein can result in lower manufacturing costs, e.g., materials, capital, and labor costs, as compared to known methods for forming sustained release bisphosphonate compositions. For example, the methods described herein can reduce the quantity of organic solvents needed to form microparticles. By reducing the quantity of organic solvent needed to form microparticles, operation and waste disposal costs can be reduced.

### BRIEF DESCRIPTION OF THE DRAWINGS

FIG. 1 is a plot of percent cumulative in vitro release of risedronate from several microparticle formulations, formed using various methods and having a targeted risedronate sodium content of 5 weight percent, versus time (in days).

FIG. 2 is a plot of mean (n=3) risedronate blood serum concentration (in nanograms/millimeter (ng/mL)) versus time (in days) post subcutaneous administration of several microparticle formulations, formed using various methods and having a targeted risedronate sodium content of 5 weight percent, to Sprague-Dawley rats with a normalized dose of 20 milligrams risedronate sodium per kilogram (based on actual risedronate sodium microparticle load).

FIG. 3 is a plot of mean (n=3) cumulative area under the curve (AUC), as a percentage of equivalent subcutaneous bolus injection, versus time (in days) post subcutaneous administration of several microparticle formulations, formed using various methods and having a targeted risedronate sodium content of 5 weight percent, to Sprague-Dawley rats with a normalized dose of 20 milligrams risedronate sodium per kilogram (based on actual risedronate sodium microparticle load).

FIG. 4 is a plot of percent cumulative in vitro release of risedronate from several microparticle formulations, formed using W/O/W and W/O/O emulsion methods and having a targeted risedronate sodium content of 5 weight percent, versus time (in days).

FIG. 5 is a plot of mean (n=3) cumulative AUC, as a percentage of equivalent subcutaneous bolus injection, versus time (in days) post subcutaneous administration of several microparticle formulations, containing a poly(d,l-lactide-co-glycolide) polymer having 50 mol % d,l-lactide, 50 mol % glycolide, formed using various methods, and having a targeted risedronate sodium content of 5 weight percent, to Sprague-Dawley rats with a normalized dose of 20 milligrams risedronate sodium per kilogram (based on actual risedronate sodium microparticle load).

FIG. 6 is a plot of mean (n=3) risedronate blood serum concentration (in ng/mL) versus time (in days) post subcutaneous administration of several microparticle formulations, each containing a poly(d,l-lactide-co-glycolide) polymer having 50 mol % d,l-lactide, 50 mol % glycolide, formed using various methods, and having a targeted risedronate sodium content of 5 weight percent, to Sprague-Dawley rats with a normalized dose of 20 milligrams risedronate sodium per kilogram (based on actual risedronate sodium microparticle load).

FIG. 7 is a plot of mean (n=3) risedronate blood serum concentration (in ng/mL) versus time (in days) post subcutaneous administration of several microparticle formulations, formed using a W/O/W emulsion method and having a targeted risedronate sodium content of 5 weight percent, to Sprague-Dawley rats with a normalized dose of 20 milligrams risedronate sodium per kilogram (based on actual risedronate sodium microparticle load).

FIG. 8 is a plot of mean (n=3) cumulative AUC, as a percentage of equivalent subcutaneous bolus injection, versus time (in days) post subcutaneous administration of several microparticle formulations, formed using a W/O/W emulsion method and having a targeted risedronate sodium content of 5 weight percent, to Sprague-Dawley rats with a normalized dose of 20 milligrams risedronate sodium per kilogram (based on actual risedronate sodium microparticle load).

FIG. 9 is a plot of mean (n=3) cumulative AUC, as a percentage of equivalent subcutaneous bolus injection, versus time (in days) post subcutaneous administration of two microparticle formulations, formed using a W/O/W emulsion method and having a targeted risedronate sodium content of 5 weight percent, to Sprague-Dawley rats with a normalized dose of 20 milligrams risedronate sodium per kilogram (based on actual risedronate sodium microparticle load).

FIG. 10A is a plot of mean (n=3) risedronate blood serum concentration (in ng/mL) versus time (in days) post subcutaneous administration of two microparticle formulations, formed using W/O/W and S/O/W emulsion methods and having a targeted risedronate sodium content of 5 weight percent, to Sprague-Dawley rats with a normalized dose of 20 milligrams risedronate sodium per kilogram (based on actual risedronate sodium microparticle load).

FIG. 10B is an exploded view of the data of FIG. 10A for about the first two days post administration of the microparticles.

FIG. 11 is a plot of mean (n=6) risedronate blood serum concentration (in ng/mL) versus time (in days) post subcutaneous administration of two MEDISORB® 6535 DL PLG LOW IV polymer microparticle formulations, formed using two W/O/W emulsion methods differing in production scale and both having a targeted risedronate sodium content of 5 weight percent, to Sprague-Dawley rats with a normalized dose of 20 milligrams risedronate sodium per kilogram (based on actual risedronate sodium microparticle load).

FIG. 12 is a plot of mean risedronate blood serum concentration (in ng/mL) versus time (in days) post subcutaneous administration of three MEDISORB® 7525 DL PLG LOW IV polymer microparticle formulations, formed using two W/O/W emulsion methods differing in production scale and both having a targeted risedronate sodium content of 5 weight percent, to Sprague-Dawley rats with a normalized dose of 20 milligrams risedronate sodium per kilogram (based on actual risedronate sodium microparticle load).

FIG. 13 is a plot of mean risedronate blood serum concentration (in ng/mL) versus time (in days) post subcutaneous administration of two MEDISORB® 7525 HIGH IV polymer microparticle formulations, formed using two W/O/W emulsion methods differing in production scale and having a targeted risedronate sodium content of 5 weight percent, to Sprague-Dawley rats with a normalized dose of 20 milligrams risedronate sodium per kilogram (based on actual risedronate sodium microparticle load).

FIG. 14 is a plot of mean risedronate blood serum concentration (in ng/mL) versus time (in days) post subcutaneous administration of two MEDISORB® 8515 DL PLG 6A polymer microparticle formulations, formed using two W/O/W emulsion methods differing in production scale and having a targeted risedronate sodium content of 5 weight percent, to Sprague-Dawley rats with a normalized dose of 20 milligrams risedronate sodium per kilogram (based on actual risedronate sodium microparticle load).

FIG. 15 is a plot of mean risedronate blood serum concentration (in ng/mL) versus time (in days) post subcutaneous administration of two MEDISORB® 100 DL 4M polymer microparticle formulations, formed using two W/O/W emulsion methods differing in production scale and having a targeted risedronate sodium content of 5 weight percent, to Sprague-Dawley rats with a normalized dose of 20 milligrams risedronate sodium per kilogram (based on actual risedronate sodium microparticle load).

FIG. 16 is a plot of mean (n=3) risedronate blood serum concentration (in ng/mL) versus time (in days) post subcutaneous administration of several MEDISORB® 6535 DL PLG LOW IV polymer microparticle formulations to Sprague-Dawley rats with a normalized dose of 20 milligrams risedronate sodium per kilogram (based on actual risedronate sodium microparticle load).

FIG. 17 is a plot of mean (n=3) cumulative AUC, as a percentage of equivalent subcutaneous bolus injection, versus time (in days) post subcutaneous administration of several MEDISORB® 6535 DL PLG LOW IV polymer microparticle formulations to Sprague-Dawley rats with a normalized dose of 20 milligrams risedronate sodium per kilogram (based on actual risedronate sodium microparticle load).

FIG. 18 is a plot of mean (n=3) risedronate blood serum concentration (in ng/mL) versus time (in days) post subcutaneous administration of several MEDISORB® 100 DL 4M polymer microparticle formulations to Sprague-Dawley rats with a normalized dose of 20 milligrams risedronate sodium per kilogram (based on actual risedronate sodium microparticle load).

FIG. 19 is a plot of mean (n=3) cumulative AUC, as a percentage of equivalent subcutaneous bolus injection, versus time (in days) post subcutaneous administration of several MEDISORB® 100 DL 4M microparticle formulations to Sprague-Dawley rats with a normalized dose of 20 milligrams risedronate sodium per kilogram (based on actual risedronate sodium microparticle load).

FIG. 20 is a plot of mean (n=3) risedronate blood serum concentration (in ng/mL) versus time (in days) post subcutaneous administration of several MEDISORB® 7525 HIGH IV polymer microparticle formulations to Sprague-Dawley rats with a normalized dose of 20 milligrams risedronate sodium per kilogram (based on actual risedronate sodium microparticle load).

FIG. 21 is a plot of mean (n=3) cumulative AUC, as a percentage of equivalent subcutaneous bolus injection, versus time (in days) post subcutaneous administration of several MEDISORB® 7525 HIGH IV microparticle formulation to Sprague-Dawley rats with a normalized dose of 20 milligrams risedronate sodium per kilogram (based on actual risedronate sodium microparticle load).

FIG. 22 is a plot of mean (n=6) risedronate blood serum concentration (in ng/mL) versus time (in days) post subcutaneous administration of a MEDISORB® 7525 HIGH IV polymer microparticle formulations to Sprague-Dawley rats using two needle gauges with a normalized dose of 10 milligrams risedronate sodium per kilogram (based on actual risedronate sodium microparticle load).

FIG. 23 is a plot of mean (n=6) cumulative AUC, as a percentage of equivalent subcutaneous bolus injection, versus time (in days) post subcutaneous administration of a MEDISORB® 7525 HIGH IV microparticle formulation to Sprague-Dawley rats using two needle gauges with a normalized dose of 10 milligrams risedronate sodium per kilogram (based on actual risedronate sodium microparticle load).

FIG. 24 is a plot of mean (n=4) cumulative area under the curve (AUC), as a percentage of equivalent subcutaneous bolus injection, versus time (in days) post subcutaneous administration of a microparticles subjected to varying amounts of gamma radiation to Sprague-Dawley rats with a normalized dose of 20 milligrams risedronate sodium per kilogram (based on actual risedronate sodium microparticle load).

The features and other details of the method of the invention will now be more particularly described with reference to the accompanying drawings and pointed out in the claims. It should be understood that the particular embodiments of the invention are shown by way of illustration and not as limitations of the invention. The principal features of this invention can be employed in various embodiments without departing from the scope of the invention.

The present invention relates to methods of forming microparticles that include a bisphosphonate and a polymer. The methods of forming microparticles can include the step of forming a water-in-oil emulsion by mixing an aqueous solution of the bisphosphonate with a combination of a biocompatible polymer and a polymer solvent. At least one aqueous liquid can be mixed with the water-in-oil emulsion to form a water-in-oil-in-water emulsion and to extract the polymer solvent from the polymer, thereby forming the microparticles.

A “microparticle,” as that term is used herein, includes a polymer having a bisphosphonate, or a salt thereof, incorporated therein. The polymer can include a biocompatible polymer such as, for example, a poly(lactic acid) or a poly(lactic acid-co-glycolic acid) copolymer. The microparticles can be used to deliver the bisphosphonate to a patient in need thereof such as, for example, in a sustained manner. The microparticles can be of any shape, for example, spherical, non-spherical or irregularly shaped, and are suitable for administration by any means (e.g., by injection such as by needle or needle-free delivery or by inhalation). The microparticles can have a particle size of less than about one millimeter, for example, ranging from about 1 micron to about 1000 microns.

In one embodiment, the microparticles are of a size suitable for injection. Microparticles suitably sized for injection range from about 10 microns or less to about 200 microns or more. In some embodiments, microparticles suitably sized for injection range from about 15, 20, 25, 30, or about 35 microns to about 65, 70, 75, 80, 85, 90, 95, or about 100 microns. For example, microparticles suitably sized for injection can range from about 40 or about 45 microns to about 50, 55, or about 60 microns.

The microparticles can be homogeneous or heterogeneous, for example, the microparticles can have a homogeneous or heterogeneous distribution of the bisphosphonate. In some embodiments, the microparticles can further include excipients such as, for example, surfactants, carbohydrates (e.g., monosaccharides and polysaccharides), release modifying agents, stabilizers, one or more additional therapeutic, prophylactic, or diagnostic agents, and any combination thereof. In other embodiments, the microparticles consist essentially of bisphosphonate and polymer.

The microparticles can be produced aseptically or terminally sterilized by gamma-irradiation such as by exposing microparticles to cobalt 60 gamma radiation. Such a terminal sterilization process can be desirable in that it can yield a sterile microparticle product without aseptic process validation. A terminally sterilized microparticle product can have the further advantages of a reduction in batch rejection due to sterility concerns and also parametric lot release. The desired range of gamma radiation exposure for a terminally sterilized product is about 15 kGy to about 45 kGy. For example, microparticles can be exposed to about 10 to about 20 kGy, e.g., 16 kGy, of gamma radiation. In other embodiments, microparticles are exposed to about 20 to about 30 kGy, e.g., 26 kGy, of gamma radiation.

As used herein, the term “particle size” refers to a number median diameter or a volume median diameter as determined by conventional particle size measuring techniques known to those skilled in the art such as, for example, laser diffraction, photon correlation spectroscopy, sedimentation field flow fractionation, disk centrifugation, electrical sensing zone method, or size classification such as sieving. The “number median diameter” reflects the distribution of particles (by number) as a function of particle diameter. The “volume median diameter” is the median diameter of the volume weighted size distribution, also referred to as D_{v,50}. The volume median diameter reflects the distribution of volume as a function of particle diameter. One example of a device that can be used to measure particle size (e.g., volume median diameter) is a Coulter LS Particle Size Analyzer (e.g., Model 130) (Beckman Coulter, Inc. Fullerton, Calif.). “Particle size” can also refer to the minimum dimension of a population of particles. For example, particles that are size classified by sieving can have a minimum dimension that is based on the size of the holes contained in the sieve.

As used herein and as generally recognized in the art, the term “water-in-oil emulsion” (“W/O emulsion”) refers to an emulsion that includes a discontinuous phase, e.g., a “water” phase, or predominantly aqueous phase, and a continuous phase, e.g., an “oil” phase, or predominantly organic liquid phase, such as a predominantly polymer solvent phase. The oil phase is at least partially immiscible with the water phase. The term “water-in-oil-in-water emulsion” (“W/O/W emulsion”) refers to an emulsion that includes an inner emulsion discontinuous phase, e.g., a “water-in-oil emulsion,” or an inner emulsion, and a continuous phase, e.g., a “water” phase, or predominantly aqueous phase. The oil phase is at least partially immiscible with the water phases.

As used herein and as generally recognized in the art, the term “solid-in-oil-in-water emulsion” (“S/O/W emulsion”) refers to an emulsion that includes a dispersion of a solid material (e.g., a bisphosphonate) in an “oil” phase, or predominantly organic liquid phase, such as a predominantly polymer solvent phase as the discontinuous phase, and a continuous phase, e.g., a “water” phase, or predominantly aqueous phase. The oil phase is at least partially immiscible with the water phase.

Likewise, as used herein and as generally recognized in the art, the term “water-in-oil-in-oil emulsion” (“W/O/O emulsion”) refers to an emulsion that includes an inner emulsion discontinuous phase, e.g., a “water-in-oil emulsion,” or an inner emulsion, and a continuous phase, e.g., an “oil” phase, or predominantly organic phase. The term “solid-in-oil-in-oil emulsion” (“S/O/O emulsion”) refers to an emulsion that includes a dispersion of a solid material (e.g., a bisphosphonate) in a first “oil” phase, or organic liquid phase, such as a predominantly polymer solvent phase as the discontinuous phase and a continuous phase, e.g., a second “oil” phase, or predominantly organic liquid phase. The first and second oil phases are at least partially immiscible with each other.

The methods of forming microparticles described herein can include the step of forming a water-in-oil emulsion by mixing an aqueous solution of the bisphosphonate with a combination of a biocompatible polymer and a polymer solvent.

Bisphosphonates are a group of synthetic pyrophosphates characterized by a “Phosphorous-Carbon-Phosphorous”-type backbone. The bisphosphonates are potent inhibitors of bone resorption and ectopic calcification. “Bisphosphonate,” as the term is used herein, includes compounds represented by Chemical Structure I: 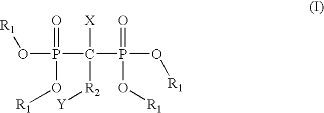
wherein, R₁ is independently, H, alkyl, aryl or heteroaryl; X is H, —OR₁ or halogen; R₂ is H, O, S, N, (CH₂)ₙ, branched alkylene, branched or straight alkenylene or alkynylene; n is an integer from about 0 to about 18; Y is H, R₁, halogen, amino, cyano or amido group; and pharmaceutically acceptable salts thereof.

As used herein, “alkyl” refers to a straight chain or branched, substituted or unsubstituted C₁-C₁₈ hydrocarbon group. Examples of suitable alkyl groups include, but are not limited to, methyl, ethyl, propyl, butyl, pentyl, isopropyl, isobutyl, and tert-butyl. As used herein, “halogen” refers to chlorine, bromine, iodine, fluorine, and astatine. The term “aryl” as used herein refers to unsubstituted and substituted aromatic hydrocarbons. The term “heteroaryl” as used herein refers to unsubstituted or substituted aryl groups wherein at least one carbon of the aryl group is replaced with a heteroatom (e.g., N, O or S). Suitable substituents, include, for example, but are not limited to, halogen, —OH, alkoxy, amino, amido, —SH, cyano, —NO₂, —COOH, —COH, —COOR₁.

Bisphosphonates suitable for use in the present invention include, but are not limited to, (1-hydroxyethylidene)bis-phosphonate (i.e., etidronate); (dichloromethylene)bis-phosphonate (i.e., clodronate); (((4-chlorophenyl)thio)-methylene)bis-phosphonate (i.e., tiludronate); (3-amino-1-hydroxypropylidene)bis-phosphonate (i.e., pamidronate); dimethyl pamidronate; (4-amino-1-hydroxybutylidene)bis-phosphonate (i.e., alendronate); (1-hydroxy-3-(methylpentylamino)propylidene)bis-phosphonate (i.e., ibandronate); (1-hydroxy-2-(3-pyridinyl)ethylidene)bis-phosphonate) (i.e., risedronate); (1-hydroxy-2-(1H-imidazole-1-yl)ethylidene)bis-phosphonate (i.e., zoledronate); (1-hydroxy-2-imidazo-(1,2-a)pyridin-3-ylethylidene)bis-phosphonate (i.e., YH 529); ((cycloheptylamino)-methylene)bis-phosphonate (i.e., icadronate); (3-(dimethylamino)-1-hydroxypropylidene)bis-phosphonate (i.e., olpadronate); (6-amino-1-hydroxyhexylidene)bis-phosphonate (i.e., neridronate); (1-hydroxy-3-(methylpentylamino)propylidene)bis-phosphonate (i.e., EB-1053); pharmaceutically acceptable salts; and combinations thereof.

A number of bisphosphonates such as alendronate sodium, risedronate sodium, pamidronate disodium, etidronate disodium, and tiludronate disodium are currently used for the treatment of moderate to severe Paget's disease and hypercalcemia associated with malignant neoplasms, treatment of osteolytic bone lesions associated with multiple myeloma and treatment of osteoporosis. In one embodiment, the bisphosphonate is selected from the group consisting of alendronate, risedronate, pamidronate, etidronate, tiludronate, pharmaceutically acceptable salts, and combinations thereof.

In one embodiment, the bisphosphonate is risedronate, a compound represented by Chemical Structure II: 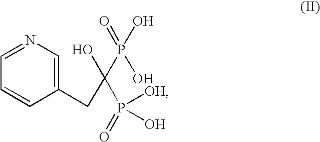
or a pharmaceutically acceptable salt thereof. For example, the bisphosphonate can be (1-hydroxy-2-(3-pyridinyl)ethylidene)bis(phosphonic acid) monosodium salt, or risedronate sodium. In one embodiment, the bisphosphonate is a hydrate.

Bisphosphonates suitable for use in the invention include those described in U.S. Pat. No. 4,705,651 to Staibano; U.S. Pat. No. 4,327,039 to Blum, et al.; U.S. Pat. Nos. 5,312,954 and 5,196,409 to Breuer, et al., U.S. Pat. No. 5,412,141 to Nugent, U.S. Pat. Nos. 4,922,007 and 5,019,651 to Kieczykowski, et al., U.S. Pat. No. 5,583,122 to Benedict, et al., U.S. Pat. No. 6,080,779 to Gasper, et al., U.S. Pat. No. 6,117,856 to Benderman, et al., U.S. Pat. No. 6,162,929 to Foricher, et al. and U.S. Pat. No. 5,885,473 to Papapoulos, et al., the entire contents of each of which are incorporated herein by reference.

The aqueous solution of the bisphosphonate can be formed by dissolving a bisphosphonate in an aqueous medium. In one embodiment, the concentration of the bisphosphonate in the aqueous solution is less than or equal to the room temperature (e.g., about 21 to about 23° C.) solubility limit of the bisphosphonate. In another embodiment, the concentration of the bisphosphonate in the aqueous solution is greater than the room temperature solubility limit of the bisphosphonate. For example, an aqueous solution containing a concentration of bisphosphonate that is greater than the room temperature solubility limit of the bisphosphonate can be prepared by heating a mixture of the bisphosphonate and an aqueous medium, e.g., water. In some embodiments, the concentration of the bisphosphonate, e.g., risedronate sodium, in the aqueous solution is at least about 50 milligrams/milliliter (mg/mL), for example, at least about 75, at least about 100 or at least about 125 mg/mL. In one embodiment, the concentration of the bisphosphonate in the aqueous solution is at least about twice the room temperature solubility limit of the bisphosphonate. In other embodiments, the concentration of the bisphosphonate in the aqueous solution can be at least about 2.5, 2.75, or at least about 3 times the room temperature solubility limit of the bisphosphonate.

The aqueous medium and/or the aqueous solution can further include an additive. For example, the aqueous solution can include a surfactant, preferably a non-ionic surfactant. The surfactant can include, but is not limited to, polyvinyl alcohol (PVA), poloxamers, polysorbates, sorbitan fatty acid esters, and polyvinylpyrrolidone (PVP). Suitable poloxamers include poloxamer 188 (poloxamer 188 includes block copolymers of ethylene oxide and propylene oxide), e.g., Pluronic F68. Suitable polysorbates include polyethylene glycol sorbitan monolaurate, e.g., polysorbate 20 such as Tween® 20 (Tween® is a trademark of ICI Americas, Inc.). Suitable sorbitan fatty acid esters include, for example, sorbitan monolaurate (Span 20), sorbitan monopalmitate (Span 40), sorbitan monostearate (Span 60), sorbitan tristearate (Span 65), sorbitan monooleate (Span 80), and sorbitan trioleate (Span 85).

The aqueous medium and/or the aqueous solution can include a surfactant in a concentration of, for example, at least about 0.05% (w/v), at least about 0.1% (w/v), at least about 0.15% (w/v), or at least about 0.2% (w/v). In one embodiment, the aqueous medium and/or the aqueous solution includes a surfactant in a concentration ranging from about 0.05 to about 0.15% (w/v) such as about 0.07 to about 0.13% (w/v), or about 0.09 to about 0.11% (w/v). For example, the aqueous medium can include a surfactant at a concentration of about 0.1% (w/v). In one embodiment, one or more other excipients such as carbohydrates, amino acids, fatty acids, and bulking agents can be added to the aqueous solution so that the excipient(s) can be present in the subsequently formed microparticles, for example, to maintain the potency of the bisphosphonate over the duration of release or to modify polymer degradation and bisphosphonate release. The excipient(s) can be suspended or dissolved in the aqueous solution. However, the microparticles described herein can provide suitable bisphosphonate release characteristics in vivo when formed without using additives such as excipients. Thus, in one embodiment, the aqueous solution consists essentially of water and the bisphosphonate.

In some embodiments, the aqueous solution further contains one or more therapeutic, prophylactic, or diagnostic agents, e.g., a biologically active agent, in addition to the bisphosphonate. Suitable additional therapeutic, prophylactic, or diagnostic agents include, but are not limited to, bone morphogenic proteins (BMPs), osteogenic proteins, parathyroid hormone (PTH), calcitonin, estrogens and selective estrogen receptor modulators (SERMs).

The mixture of the bisphosphonate and the aqueous medium can be heated to increase the concentration of the bisphosphonate in a given quantity of aqueous liquid. In one embodiment, the mixture of the bisphosphonate and the aqueous liquid are heated to a temperature higher than room temperature (e.g., about 21 to about 23° C.), such as to at least about 50, 55, 60, 65, 70, 75, 80, 85, 90 or to at least about 95° C. In one embodiment, the temperature of the aqueous solution is at least about 50° C. In another embodiment, the temperature of the aqueous solution is at least about 75° C., e.g., about 75° C. to about 85° C. such as about 80° C.

In one embodiment, the aqueous solution is at about room temperature (e.g., about 21 to about 23° C.) for subsequent use in forming the water-in-oil emulsion. For example, a mixture of a bisphosphonate and an aqueous medium can be heated to form an aqueous solution and the aqueous solution is then cooled, e.g., to about room temperature, prior to forming a water-in-oil emulsion. The aqueous solution can be a supersaturated solution of the bisphosphonate. In other embodiments, the aqueous solution is an unsaturated or a saturated solution of the bisphosphonate.

As used herein, a “solution” is a mixture of one or more substances, referred to as the solute(s), dissolved in one or more other substances, referred to as the solvent(s).

The combination of a biocompatible polymer and a polymer solvent can be formed, for example, by mixing a biocompatible polymer with an appropriate solvent. Suitable biocompatible polymers include biodegradable and non-biodegradable polymers and blends and copolymers thereof, as described herein. A polymer is biocompatible if the polymer and any degradation products of the polymer are non-toxic to the patient and also possess no significant deleterious or untoward effects on the patient's body, such as a significant immunological reaction at a site of administration.

“Biodegradable,” as defined herein, means the composition will degrade or erode in vivo to form smaller chemical species. Degradation can result, for example, by enzymatic, chemical and physical processes. Suitable biocompatible, biodegradable polymers include, for example, polylactides, polyglycolides, poly(lactide-co-glycolide)s, poly(lactic acid)s, poly(glycolic acid)s, polycarbonates, polyesteramides, polyanydrides, poly(amino acid)s, polyorthoesters, polydioxanones, poly(alkylene alkylate)s, copolymers or polyethylene glycol and polyorthoester, biodegradable polyurethane, blends thereof, and copolymers thereof.

Suitable biocompatible, non-biodegradable polymers include non-biodegradable polymers such as, for example, polyacrylates, polymers of ethylene-vinyl acetates and other acyl substituted cellulose acetates, non-biodegradable polyurethanes, polystyrenes, polyvinylchloride, polyvinyl flouride, poly(vinyl imidazole), chlorosulphonate polyolefins, polyethylene oxide, blends thereof, and copolymers thereof, such as PLG-co-EMPO described in U.S. patent application Ser. No. 09/886,394 entitled “Functionalized Degradable Polymer” and filed on Jun. 22, 2001, the entire contents of which is hereby incorporated by reference.

Further, the terminal functionalities or pendant groups of the biocompatible polymers can be modified, for example, to modify hydrophobicity, hydrophilicity and/or to provide, remove or block moieties which can interact with the biologically active agent via, for example, ionic or hydrogen bonding.

In one embodiment, the biocompatible polymer is at least one member selected from the group consisting of polylactides, polyglycolides, poly(lactide-co-glycolide)s, poly(lactic acid)s, poly(glycolic acid)s, polycarbonates, polyesteramides, polyanhydrides, poly(amino acid)s, polyorthoesters, polyacetals, polycyanoacrylates, polyetheresters, polycaprolactone, polydioxanones, poly(alkylene alkylate)s, polyurethanes, and blends and copolymers thereof.

In a preferred embodiment of the present invention, the polymer used is a poly(lactic acid-co-glycolic acid) (“PLG”) copolymer. The poly(lactic acid-co-glycolic acid) polymer includes d-, l-, or racemic forms of the polymer. For example, in some embodiments, poly(d,l-lactic acid-co-glycolic acid) can be used. In some embodiments, the poly(lactic acid-co-glycolic acid) includes an acid end group, e.g., a free carboxyl end group. In other embodiments, the poly(lactic acid-co-glycolic acid) contains an ester end group, e.g., an alkyl ester end group such as a methyl ester end group or a lauryl ester end group.

In some embodiments, the molar ratio of a lactide component to a glycolide component of the polymer can range from about 50:50 to about 100:0. For example, the polymer can be a poly(lactide-co-glycolide) which has a lactide to glycolide ratio of about 65:35 to about 85:15, e.g., the polymer can have a lactide to glycolide ratio of about 65:35, 75:25 or 85:15. In a preferred embodiment, the method of forming the microparticles includes the step of forming a water-in-oil emulsion by mixing an aqueous solution of the bisphosphonate with a combination of a poly(lactide-co-glycolide) polymer and a polymer solvent, wherein the molar ratio of the lactide component to the glycolide component in the polymer is at least about 65:35. For example, the molar ratio of the lactide component to the glycolide component of the polymer can be about 65:35 to about 85:15.

The inherent viscosity of the polymer, measured in chloroform at 25° C., can range, for example, from about 0.3 to about 0.9 deciliters/gram (dL/g). In one embodiment, the inherent viscosity of the polymer is no more than about 0.65 dL/g. In another embodiment, the inherent viscosity of the polymer is about 0.8 to about 0.9 dL/g.

Acceptable molecular weights for biocompatible polymers used in this invention can be determined by a person of ordinary skill in the art taking into consideration factors such as the desired polymer degradation rate, physical properties such as mechanical strength, and the rate of dissolution of polymer in the solvent. Typically, an acceptable range of molecular weight is from about 2,000 daltons to about 2,000,000 daltons.

Polymer suitable for use in the present invention include, but are not limited to, MEDISORB® 5050 PLG 4A, MEDISORB® 5050 DL PLG 5A, MEDISORB® 5050 DL PLG HIGH IV, MEDISORB® 8515 DL PLG 6A, MEDISORB® 100 DL 4M, MEDISORB® 6535 DL PLG LOW IV, MEDISORB® 6535 DL PLG HIGH IV, MEDISORB® 7525 DL PLG LOW IV, all of which are commercially available from Lakeshore Biomaterials, Inc. (Birmingham, Ala.) (MEDISORB is a trademark of Alkermes, Inc.). MEDISORB® 7525 DL PLG HIGH IV polymer, a poly(d,l-lactide-co-glycolide) polymer having 75 mol % d,l-lactide, 25 mol % glycolide, a lauryl ester end group, and an IV of typically about 0.8 to about 0.9 dL/g, is also suitable for use in the present invention and can be obtained from various manufacturers, e.g., Alkermes, Inc., by special order.

The combination of the biocompatible polymer and the polymer solvent can include a polymer solvent selected from a variety of common organic solvents including halogenated aliphatic hydrocarbons such as chloroform, methylene chloride, methylchloroform and the like; aromatic hydrocarbon compounds; halogenated aromatic hydrocarbon compounds; cyclic ethers such as tetrahydrofuran and the like; alcohols, water, acetone, ethyl acetate and the like. The polymer solvent is typically a material which will at least partially dissolve the polymer, which is substantially chemically inert with respect to the bisphosphonate, and which is substantially immiscible with the aqueous phase(s) of the emulsion(s). In one embodiment, the polymer solvent is represented by the chemical structure, R₃COOR₄, wherein R₃ and R₄ are, independently, alkyl groups having from about 1 to about 4 carbon atoms. For example, the polymer solvent can be ethyl acetate.

In one embodiment, the concentration of the polymer in the combination of the polymer and polymer solvent can range from about 1 to about 50 weight percent polymer. For example, the concentration of the polymer in the combination can range from about 5 to about 40, about 10 to about 25, or about 12 to about 20 weight percent polymer such as about 12 to about 19 or about 15 to about 18 weight percent polymer.

In some embodiments, the combination of the polymer and polymer solvent further contains one or more therapeutic, prophylactic, or diagnostic agents, e.g., a biologically active agent, in addition to the bisphosphonate. Suitable additional therapeutic, prophylactic, or diagnostic agents include, but are not limited to, bone morphogenic proteins (BMPs), osteogenic proteins, parathyroid hormone (PTH), calcitonin, estrogens and selective estrogen receptor modulators (SERMs).

The aqueous solution of the bisphosphonate and the combination of the biocompatible polymer and the polymer solvent, described supra, can be mixed to form the water-in-oil emulsion. Any of the techniques known in the art for forming a water-in-oil emulsion can be used. For example, forming the water-in-oil emulsion can include mixing the aqueous solution with the combination of the polymer and the polymer solvent using rotor-stator mixing or sonication. In one embodiment, both the aqueous solution of the bisphosphonate and the combination of the biocompatible polymer and the polymer solvent are at substantially the same temperature, e.g., room temperature such as about 21° C. to about 23° C., just prior to the time of mixing or at the time of mixing. In another embodiment, the temperature of the aqueous solution of the bisphosphonate is higher than the temperature of the combination of the biocompatible polymer and the polymer solvent just prior to the time of mixing or at the time of mixing. For example, the aqueous solution of the bisphosphonate can be at least about 50, 55, 60, 65, 70, 75, 80, 85 or to at least about 90° C. In one embodiment, the temperature of the aqueous solution is at least about 75° C., e.g., about 75° C. to about 85° C. such as about 80° C. In one embodiment, the combination of the biocompatible polymer and the polymer solvent is at room temperature just prior to the time of mixing or at the time of mixing. Alternatively, the temperature of the combination of the biocompatible polymer and the polymer solvent can be higher than room temperature just prior to the time of mixing or at the time of mixing with the aqueous solution.

The quantity of aqueous solution mixed with the combination of the polymer and the polymer solvent will vary depending upon the targeted loading of bisphosphonate in the microparticles, among other factors. For example, about 1.5 to about 5 mL of an about 150 to about 50 mg/mL aqueous solution of bisphosphonate can be mixed with about 20 to 40 mL of an about 5 to about 20 weight percent polymer combination of polymer and polymer solvent to produce microparticles having a targeted load of 5 weight percent bisphosphonate. In one embodiment, a quantity of aqueous solution having about 5 g bisphosphonate is mixed with a combination of polymer and polymer solvent having about 95 g polymer to ultimately produce about 100 g of microparticles with an about 5 weight percent bisphosphonate load.

The methods for forming microparticles described herein can also include the step of mixing at least one aqueous liquid with the water-in-oil emulsion to form a water-in-oil-in-water emulsion and to extract the polymer solvent from the polymer, thereby forming the microparticles. Suitable static mixers and methods for their use are described in U.S. Pat. No. 5,654,008, issued to Herbert, et al., on Aug. 5, 1997, and U.S. Pat. No. 6,331,317, issued to Lyons, et al., on Dec. 18, 2001, the entire contents of both of which are incorporated herein by reference. In one embodiment, the static mixer is a 0.25 inch (in) (about 0.64 centimeter (cm)) outside diameter, 34 element, static mixer constructed of 316 stainless steel, for example, static mixer model no. 04669-60 obtainable from Cole-Parmer Instrument Co. (Vernon Hills, Ill.). Another static mixer that can be used is an Interfacial Surface Generator (ISG). For example, an 1/8 inch ISG or a 1/16 inch ISG can be used to practice the methods of the present invention. One supplier of ISG equipment is Ross Engineering (Savannah, Ga.).

A controlled and steady flow through the static mixer can be achieved by one of ordinary skill in the art. The rate of flow through the static mixer can be varied to achieve desired microparticle attributes. Suitable methods for achieving desired flow through the static mixer include the use of pressure transfer, peristaltic pumps, gear pumps, and positive displacement rotary lobe pumps. In one embodiment, a Watson-Marlow peristaltic pump is used. Another pump that may be used is a Cole-Parmer gear pump.

In one embodiment, a 0.25 in (about 0.64 cm) outside diameter, 12 in long (about 30.5 cm) static mixer is used, and an aqueous liquid stream at a flow rate of about 200 to about 6000 millimeters/minute (mL/min), e.g., about 300 to about 3000 mL/min or about 700 to about 1500 mL/min, and a water-in-oil emulsion stream at a flow rate of about 20 to about 1500 mL/min, e.g., about 70 to about 140 mL/min, are directed into the static mixer for a combined stream flow rate of about 220 to about 7500 mL/minute. In one embodiment, the flow rates can be selected considering the geometry of the static mixer and the viscosity of the streams to influence the size of the produced microparticles. For example, high flow rates, e.g., about 700 to about 1500 mL/min used with the above-described 12 inch static mixer, can be used to produce microparticles that are suitable for administration through small gauge needles or through needle-free injection devices.

The aqueous liquid can be water or an aqueous solution. In one embodiment, the aqueous liquid includes a surfactant such as polyvinyl alcohol (PVA). The concentration of the surfactant in the aqueous liquid can range from about 0.5 to about 5 weight percent such as about 1 to about 3 weight percent. In one particular embodiment, the aqueous liquid contains about 1 weight percent surfactant such as, for example, PVA. In one embodiment, the aqueous solution can contain a polymer solvent as described supra. The concentration of the polymer solvent in the aqueous liquid can range from about 5 to about 10 weight percent. In one particular embodiment, the aqueous liquid contains about 6 to about 7 weight percent polymer solvent such as, for example, ethyl acetate. In one embodiment, the aqueous liquid is saturated with the polymer solvent that is used to form the oil-in-water emulsion, supra. The aqueous liquid can include both a surfactant and a polymer solvent. For example, the aqueous liquid can contain about 1 weight percent surfactant and about 6 to about 7 weight percent polymer solvent.

In one embodiment, the step of mixing at least one aqueous liquid with the water-in-oil emulsion includes mixing an aqueous liquid with the water-in-oil emulsion to form a water-in-oil-in-water emulsion and then mixing the water-in-oil-in-water emulsion with an aqueous liquid extraction medium. The aqueous liquid extraction medium can be contained, for example, in a quench tank. In one embodiment, the water-in-oil-in-water emulsion is formed using a static mixer which discharges the emulsion into a quench tank containing the aqueous liquid extraction medium, e.g., a stirred quench tank containing the aqueous liquid extraction medium. In another embodiment, the aqueous liquid extraction medium is mixed with the emulsion in a continuous process whereby polymer solvent is continuously extracted from the polymer. For example, the aqueous liquid extraction medium can be supplied to a process wherein the water-in-oil-in-water emulsion is continuously formed and the polymer solvent is continuously extracted from the polymer.

Typically, the liquid aqueous extraction medium is water but can include aqueous solutions such as aqueous solutions of sodium chloride, polyoxyethylene sorbitan monolaureate and/or polyoxyethylene sorbitan monooleate. The liquid aqueous extraction medium can include any of the liquid aqueous extraction media known in the art. Preferably, the liquid aqueous extraction medium has a capacity for the polymer solvent contained in the water-in-oil-in-water emulsion of at least about 1 weight percent, for example, at least about 5 to about 10 weight percent or at least about 6 to about 7 weight percent, when capacity is measured at room temperature (e.g., about 21 to about 23° C.).

In preferred embodiments, at least about 90 weight percent of the polymer solvent is extracted from the polymer to form the microparticles. For example, in some embodiments, at least about 95, 97, or at least about 99 weight percent of the polymer solvent is extracted from the polymer to form the microparticles. The residual polymer solvent in the microparticles following mixing the water-in-oil emulsion with the at least one aqueous liquid is preferably less than about 10 weight percent such as less than about 5, 3 or less than about 1 weight percent.

The present invention includes a method of forming microparticles that include a bisphosphonate and a polymer wherein the method includes: (a) forming a water-in-oil emulsion by mixing an aqueous solution of the bisphosphonate with a combination of a biocompatible polymer and a polymer solvent, wherein the concentration of the bisphosphonate in the aqueous solution is greater than the room temperature solubility limit of the bisphosphonate; (b) forming a water-in-oil-in-water emulsion by mixing a first aqueous liquid with the water-in-oil emulsion; and (c) extracting the polymer solvent from the polymer into a second aqueous liquid, thereby forming the microparticles. In one embodiment, forming the water-in-oil-in-water emulsion includes mixing the water-in-oil emulsion with the first aqueous liquid, e.g., water or a water solution such as an aqueous surfactant solution, in a static mixer as described supra. The second aqueous liquid can include an aqueous liquid extraction medium, also described supra.

The present invention includes a method of forming microparticles that include a bisphosphonate and a polymer that comprises the steps of: (a) preparing an aqueous mixture of the bisphosphonate and a surfactant; (b) forming a water-in-oil emulsion by mixing the aqueous mixture with a combination of a biocompatible polymer and a polymer solvent; (c) forming a water-in-oil-in-water emulsion by mixing the water-in-oil emulsion with an aqueous liquid; and (d) removing the polymer solvent from the polymer, thereby forming the microparticles. For example, the polymer solvent can be removed from the polymer by extracting the polymer solvent using an aqueous liquid, e.g., a liquid aqueous extraction medium, described supra. In another embodiment, the polymer solvent is removed from the polymer using any of the techniques known in the art for removing a solvent from a polymer to form microparticles. For example, the polymer solvent can be removed from the polymer by liquid evaporation or sublimation.

The present invention also includes a method of forming microparticles using a solid-in-oil-in-water emulsion process. In one embodiment, a method of forming microparticles that include a bisphosphonate and a polymer comprises the steps of: (a) forming a bisphosphonate suspension in a combination consisting essentially of a biocompatible polymer and a polymer solvent; and (b) mixing at least one aqueous liquid with the bisphosphonate suspension to form a solid-in-oil-in-water emulsion and to extract the polymer solvent from the polymer, thereby forming the microparticles. The bisphosphonate suspension can be produced by dispersing a bisphosphonate in a combination of a biocompatible polymer and a polymer solvent. In one embodiment, an ultra sonication probe, e.g., Sonics and Materials, Inc., Model No. CV17 (Danbury, Conn.); a rotor-stator homogenizer, e.g., an IKA Model No. T25S6 rotor/stator homogenizer (IKA Works USA, Wilmington, N.C.); or a high pressure homogenizer, e.g., an Avestin high pressure homogenizer, Model C-5 (Ottawa, Canada), is used to disperse the bisphosphonate in the combination of the biocompatible polymer and polymer solvent.

In one embodiment, the bisphosphonate is milled prior to being suspended in the combination of the biocompatible polymer and polymer solvent. For example, a Standard Micron-Master Mill, e.g., a 1 inch model, (Jet Pulverizer Co., Moorestown, N.J.) can be used to mill the bisphosphonate. The bisphosphonate can be milled, for example, to a mean particle size of less than about 15 microns such as to a mean particle size of less than about 10, 5, or less than about 3 microns.

Alternatively, the bisphosphonate can be prepared, prior to being suspended in the combination of the biocompatible polymer and polymer solvent, by a process that includes lyophilization. Lyophilized risedronate can be prepared by methods known in the art such as bulk freeze drying, spray drying, spray-freeze drying, rotary evaporation vacuum drying, and supercritical fluid drying and those described in U.S. Pat. No. 6,284,283 issued to Costantino, et al., on Sep. 4, 2001, incorporated herein by reference in its entirety. Spray-freeze drying in particular is suitable for production of dried solids that, according to the processing conditions, can yield powders having micron to sub-micron particle sizes, for instance, mean particle sizes of less than 10 microns (e.g., Costantino, et al., U.S. Pat. No. 6,284,283). For example, lyophilized risedronate can be prepared by spraying an aqueous solution of risedronate (e.g., 1 to 5 mg/ml concentration) into a freezing medium (e.g., liquid nitrogen) using an atomization technique (e.g., single fluid, high pressure nozzle), transferring the frozen slurry into a container (e.g., Lyoguard trays, W. L. Gore and Associates, Del.), and drying the frozen slurry in a lyophilizer. Suitable lyophilizers are well known in the art (e.g., suitable models are available from FTS Systems, Stone Ridge, N.Y.). The lyophilized risedronate can be suitable for processing to produce microparticles by a variety of pharmaceutical processing methods such as described herein.

At least one aqueous liquid and the bisphosphonate suspension can be mixed to form a solid-in-oil-in-water emulsion and to extract the polymer solvent from the polymer. In one embodiment, an aqueous liquid and the bisphosphonate suspension are mixed using a static mixer as described supra. The polymer solvent can be extracted from the polymer using an aqueous liquid such as a liquid extraction medium as described supra.

The methods of the present invention can also include the step of isolating the microparticles. For example, the microparticles can be separated, e.g., filtered, from liquids, e.g., aqueous liquid(s), in which they are formed or contained. In one aspect of the method, the microparticles are formed by mixing at least one aqueous liquid with the water-in-oil emulsion to form a water-in-oil-in-water emulsion and to extract the polymer solvent from the polymer. The microparticles can then be isolated from the aqueous liquid. For example, in one embodiment, a water-in-oil-in-water emulsion is transferred to a quench tank containing an aqueous liquid extraction medium. The microparticles formed in the quench tank can be isolated by collecting the microparticles from the aqueous liquid extraction medium, e.g., by filtering the microparticles from the liquid contained in the quench tank. For example, the aqueous liquid containing the microparticles can be directed through a sieve, thereby separating the microparticles from the aqueous liquid. In one instance, a filter dryer can be used to filter and dry the microparticles. Sources for suitable filter dryers or dryer components include Martin Kurz & Co., Inc. (Mineola, N.Y.), Pope Scientific Inc. (Saukville, Wis.), and National Filter Media Corporation (Salt Lake City, Utah).

Typically, the collected microparticles can contain residual polymer solvent, residual aqueous liquid and/or other residual substances from the microparticle formation process (e.g., residual surfactants). In one embodiment, the step of isolating the microparticles includes the step of washing the collected microparticles. The collected microparticles can be washed using, for example, deionized water.

In one embodiment, residual wash water, residual polymer solvent, residual aqueous liquid, and/or other residual substances are then removed by drying the microparticles, for example, by using lyophilization or vacuum drying. In one instance, a filter dryer can be used to dry the microparticles. Sources for suitable filter dryers or dryer components include Martin Kurz & Co., Inc. (Mineola, N.Y.), Pope Scientific Inc. (Saukville, Wis.), and National Filter Media Corporation (Salt Lake City, Utah). Alternatively, a freeze/filter dryer similar to that described in U.S. patent application Ser. No. 10/304,058, filed on Nov. 26, 2002, entitled “Method and Apparatus for Filtering and Drying a Product,” incorporated in its entirety herein by reference, can be used to isolate the microparticles.

The present invention includes the use of continuous, batch, and semi-batch processes, and combinations thereof, to perform the methods described supra.

The present invention also relates to microparticles that are formed by the methods described herein. The microparticles include a biocompatible polymer such as, for example, poly(lactic acid) or a poly(lactic acid-co-glycolic acid) copolymer, and a bisphosphonate. In some embodiments, excipients, e.g., carbohydrates, amino acids, fatty acids, and bulking agents, can be present in the microparticles, for example, to maintain the potency of the bisphosphonate over the duration of release or to modify polymer degradation and bisphosphonate release. However, some excipients can increase initial burst of bisphosphonate from the microparticles upon administration (e.g., by increasing water uptake) and the microparticles described herein can provide suitable bisphosphonate release characteristics in vivo when formed without using excipients. Thus, in other embodiments, the microparticles consist essentially of the bisphosphonate and the biocompatible polymer.

The microparticles described herein can contain from about 0.01% (w/w) to about 30% (w/w) of the bisphosphonate (based on dry weight of the microparticles). The amount of bisphosphonate loading can vary depending upon the desired effect of the bisphosphonate, the planned release levels, and the time span over which the bisphosphonate is to be released. A preferred range of bisphosphonate loading is about 0.1% (w/w) to about 25% (w/w), for example, about 0.5% (w/w) to about 15% (w/w), or about 1% (w/w) to about 10% (w/w), e.g., about 1% (w/w) to about 5% (w/w), about 5% (w/w) to about 10% (w/w), about 3% (w/w) to about 8% (w/w), or about 4% (w/w) to about 6% (w/w). In some embodiments, the bisphosphonate loading ranges from about 2% (w/w) to about 6% (w/w) or from about 3% (w/w) to about 5% (w/w). For example, in one embodiment, the bisphosphonate is risedronate sodium and the risedronate sodium can be present in the microparticles at a concentration ranging from about 3% (w/w) to about 6% (w/w).

The present invention includes microparticles consisting essentially of a biocompatible polymer and at least about 3 weight percent of risedronate or a salt thereof. In one embodiment, the microparticles have an in vitro 24-hour risedronate release of less than about 15 weight percent, e.g., less than about 10 or less than about 5 weight percent, in a phosphate buffered saline composition at 37° C. The microparticles, upon administration to a patient, also can have an in vivo duration of risedronate release from the microparticles of at least about 30 days such as at least about 45 days, 60, 75, or at least about 90 days.

The present invention also includes microparticles consisting essentially of a bisphosphonate and a biocompatible polymer wherein the microparticles have an in vitro 24-hour cumulative bisphosphonate release of less than about 15 weight percent. In one embodiment, the microparticles can have an in vitro 24-hour bisphosphonate (e.g., risedronate) release of less than about 10 or less than about 5 weight percent. The in vitro 24-hour bisphosphonate release can be, for example, in a buffered composition such as in a phosphate buffered saline composition at 37° C. containing 0.02 weight percent polysorbate 20.

The present invention also includes microparticles consisting essentially of a bisphosphonate and a biocompatible polymer. In some embodiments, the microparticles consisting essentially of a bisphosphonate and a biocompatible polymer also contain residual quantities of materials used in the production of the microparticles. For example, in some embodiments, the microparticles consisting essentially of a bisphosphonate and a biocompatible polymer also contain residual quantities of water, solvents, and surfactants, among other materials. In one embodiment, the microparticles can cause a reduced local site reaction in vivo upon parenteral administration to a patient as compared to a local site reaction caused by a parenteral administration to the patient of a bisphosphonate not formed into microparticles with a biocompatible polymer. The local site reaction can include, but is not limited to, inflammation (e.g., foreign body inflammation, mixed cell inflammation, swelling, redness, and/or welting) and/or exudation. In one aspect of the invention, the microparticles can cause a local site reaction in vivo upon parenteral administration to a patient that is substantially similar to a local site reaction caused by placebo microparticles that include the biocompatible polymer. In one embodiment, the microparticles can have clinically acceptable local tolerability in vivo upon administration to a patient. For example, the microparticles can cause a local site reaction in vivo upon parenteral administration to a patient that is substantially similar to a local site reaction caused by placebo microparticles that include the biocompatible polymer. In another embodiment, the microparticles can cause a local site reaction in vivo upon parenteral administration to a patient that is substantially reduced as compared to a local site reaction caused by a parenteral administration to the patient of a bisphosphonate not formed into microparticles with a biocompatible polymer. In one embodiment, the microparticles can have clinically acceptable local tolerability in vivo upon administration to a human patient.

The present invention also relates to a pharmaceutical composition for the administration of a bisphosphonate. The pharmaceutical composition includes the microparticles of the present invention having a therapeutically effective amount of a bisphosphonate contained therein. The microparticles and microparticle-containing pharmaceutical compositions of the present invention can provide sustained release of the bisphosphonate contained therein. Thus, the microparticles described herein can be used to provide a therapeutically, prophylactically, and/or diagnostically effective amount of the bisphosphonate to a patient for a sustained period. The microparticles formed by the method of the present invention can provide increased therapeutic, prophylactic, and/or diagnostic benefits by reducing fluctuations of the bisphosphonate concentration in blood, by providing a more desirable release profile, and/or by potentially lowering the total amount of bisphosphonate needed to provide a therapeutic, prophylactic, and/or diagnostic benefit without the need for additional components.

As used herein, a “therapeutically effective amount,” a “prophylactically effective amount,” and a “diagnostically effective amount” refer to the amount of the sustained release composition needed to elicit the desired therapeutic, prophylactic or diagnostic biological response following administration of the microparticles or a microparticle-containing pharmaceutical composition to a patient. “Patient,” as that term is used herein, refers to the recipient of bisphosphonate therapy. Mammalian and non-mammalian patients are included. In a specific embodiment, the patient is a mammal, such as a human, canine, murine, feline, bovine, ovine, swine or caprine. In a preferred embodiment, the patient is a human.

“Sustained release,” as that term is used herein, is a release of the bisphosphonate from the microparticles or from a pharmaceutical composition that includes the microparticles which occurs over a period which is longer than the period during which a biologically significant amount of the bisphosphonate would be available following direct administration of the bisphosphonate, e.g., a solution or suspension of the bisphosphonate. In one embodiment, a sustained release is a release of the bisphosphonate which occurs over a period of at least about one day such as, for example, at least about 2, 4, 6, 8, 10, 15, 20, 30, 60, or at least about 90 days. A sustained release of the bisphosphonate can be a continuous or a discontinuous release, with relatively constant or varying rates of release. The continuity of release and level of release can be affected by the type of polymer composition used (e.g., monomer ratios, molecular weight, block composition, and varying combinations of polymers), bisphosphonate loading, selection of excipients to produce the desired effect, and/or methods of microparticle production.

“Sustained release” is also referred to in the art as “modified release,” “prolonged release,” “long acting release (‘LAR’),” or “extended release.” “Sustained release,” as used herein, also encompasses “sustained action” or “sustained effect.” “Sustained action” and “sustained effect,” as those terms are used herein, refer to an increase in the time period over which the bisphosphonate performs its therapeutic, prophylactic and/or diagnostic activity as compared to an appropriate control. “Sustained action” is also known to those experienced in the art as “prolonged action” or “extended action.”

Without being bound by a particular theory, it is believed that the release of the bisphosphonate from the microparticles can occur by two different mechanisms. First, the bisphosphonate can be released by diffusion through aqueous filled channels generated in the polymer matrix, such as by the dissolution of the bisphosphonate, or by voids created by the removal of the polymer solvent during the preparation of the microparticles. A second mechanism can be the release of the bisphosphonate due to degradation of the polymer. The rate of polymer degradation can be controlled by changing polymer properties that influence the rate of hydration of the polymer. These properties include, for instance, the ratio of different constituent monomers, such as lactide and glycolide; the use of an isomer of a monomer, e.g., a l-isomer, instead of a racemic mixture; and the molecular weight of the polymer. These properties can affect hydrophilicity and crystallinity, which can control the rate of hydration of the polymer.

By altering the properties of the polymer, the contributions of diffusion and/or polymer degradation to bisphosphonate release can be controlled. For example, increasing the glycolide content of a poly(lactide-co-glycolide) polymer and decreasing the molecular weight of the polymer can enhance the hydrolysis of the polymer and thus, provide an increased bisphosphonate release due to polymer erosion.

Preferably, the microparticles formed as described herein contain a substantial population of microparticles that are administrable to a patient. These microparticles suitable for administration can be used to prepare pharmaceutical compositions of the microparticles. The pharmaceutical compositions described herein also can include microparticles selected from a general microparticle population using techniques well-known in the art. For example, microparticles that are unsuitably sized for administration to a patient (e.g., by injection) can be size-separated from microparticles that are suitable for administration, thereby producing an administrable microparticle population. For example, the microparticles having particle sizes suitable for an injectable pharmaceutical composition can be separated from microparticles that are too large for practical injection. In one embodiment, a screen or sieve can be used to size-separate the microparticles.

The present invention also includes a method of mixing microparticles, as described herein, and a physiologically acceptable diluent, thereby forming a pharmaceutical composition of microparticles. In one embodiment, injectable microparticles are mixed with a physiologically acceptable diluent to form an injectable pharmaceutical composition.

In addition to a physiologically acceptable diluent, the pharmaceutical compositions described herein may also include other pharmaceutically acceptable excipients such as, for example, stabilizers and delivery vehicles. Pharmaceutically acceptable excipients can be selected by one of ordinary skill in the art without undue experimentation. Compositions for the administration of microparticles are described, for example, in U.S. Pat. No. 6,495,164, issued to Ramstack, et al., on Dec. 17, 2002, the contents of which are incorporated herein by reference. One example of a suitable physiologically acceptable diluent is 3% carboxymethylcellulose (low viscosity) and 0.1% TWEEN® 20 in 0.9% aqueous sodium chloride. Other suitable physiologically acceptable diluents include saline, sorbitol solutions and oil formulations.

In one embodiment, one or more excipients can be mixed with the microparticles or can be constituents of a pharmaceutical composition. For example, an excipient can be blended with the microparticles prior to the size-separation of microparticles unsuitable for administration. Thus, excipient particles unsuitable for administration can also be removed from the mixture of microparticles and excipient. In another embodiment, an excipient, suitably sized for administration is blended with an administrable microparticle population prior to formation of a pharmaceutical composition or is blended with a pharmaceutical composition containing microparticles.

Suitable excipients include, for example, carbohydrates, amino acids, fatty acids, surfactants, and bulking agents. Such excipients are known to those of ordinary skill in the art. An acidic or a basic excipient is also suitable. The amount of excipient used is based on its ratio to the bisphosphonate, on a weight basis. For amino acids, fatty acids and carbohydrates, such as sucrose, trehalose, lactose, mannitol, dextran and heparin, the ratio of carbohydrate to bisphosphonate, can be between about 1:10 and about 20:1. For surfactants, the ratio of surfactant to bisphosphonate can be between about 1:1000 and about 2:1. Bulking agents typically include inert materials. Suitable bulking agents are known to those of ordinary skill in the art.

Pharmaceutical compositions containing the microparticles of the present invention can also include one or more therapeutic, prophylactic, or diagnostic agents, e.g., a biologically active agent, in addition to the bisphosphonate present in the microparticles. Suitable additional therapeutic, prophylactic, or diagnostic agents include, but are not limited to, bone morphogenic proteins (BMPs), osteogenic proteins, parathyroid hormone (PTH), calcitonin, estrogens and selective estrogen receptor modulators (SERMs).

The microparticles described herein can be administered to a patient as microparticles or can be formed into another form for administration such as a film, pellet, rod, filament, cylinder, disc, or wafer. For example, the microparticles can be agglomerated and/or compressed into one of these alternative forms. The pharmaceutical composition can include one or more of these alternative microparticle forms. In one embodiment, the microparticles can be formed into an implantable pharmaceutical composition such as a mass of the microparticles. For example, in one embodiment, the microparticles can be mechanically compressed to form an implantable mass of microparticles.

In one embodiment, the microparticles are administered to a patient via injection. Microparticles suitably sized for administration by injection or contained in a pharmaceutical composition for administration by injection are referred to herein as “injectable microparticles.” In one embodiment, the injectable microparticles can have a particle size from about 1 micron to about 1000 microns. For example, the injectable microparticles can have a particle size of less than or equal to about 1000 microns such as less than or equal to about 500, 400, 300, 200, 150, 125, 115, 110, 105, 100, 90, 80, 70, 60, 50, 40 or less than or equal to about 30 microns.

The desired injectable microparticles' particle size can be chosen for compatibility with the device used to administer the microparticles to a patient. A device used to administer the microparticles to a patient via injection can be selected based on such factors as the injection type, the location of injection, the composition of the injected materials, and the volume of injection. For example, the device used to administer the microparticles can be a syringe equipped with a needle.

The present invention further relates to a method for treating a patient in need of therapy that includes the step of administering to the patient a therapeutically effective amount of the microparticles that include a bisphosphonate and a polymer, as described herein. The microparticles and microparticle-containing pharmaceutical compositions described herein can be administered in vivo, for example, to a human or to an animal, orally or parenterally such as by injection, implantation (e.g., subcutaneously, intramuscularly, intraperitoneally, intracranially, and intradermally), administration to mucosal membranes (e.g., intranasally, intravaginally, intrapulmonary, buccally or by means of a suppository), or by in situ delivery (e.g., by enema or aerosol spray) to provide the desired dosage of bisphosphonate based on the known parameters for treatment of any given medical condition. In a particular embodiment, administration of the microparticles described herein can be to a joint, for example, the articular space of a joint. For example, the microparticles can be administered to the articular space of a knee, shoulder, ankle or hip. The microparticles can be also be administered onto or into a bone. Other methods of administering microparticles to a patient that include a polymer and a bisphosphonate, suitable for use with the microparticles of the present invention, are described in U.S. Pat. No. 6,558,702, issued to Dasch, et al., on May 6, 2003, the entire contents of which are incorporated herein in their entirety. In some embodiments, one or more therapeutic, prophylactic, or diagnostic agents are co-administered with the microparticles. Co-administered agents can be administered to a patient contemporaneously with delivery of the microparticles or can be administered to a patient prior to or following administration of the microparticles. Suitable additional therapeutic, prophylactic, or diagnostic agents can include, but are not limited to, bone morphogenic proteins (BMPs), osteogenic proteins, parathyroid hormone (PTH), calcitonin, estrogens and selective estrogen receptor modulators (SERMs).

The microparticles and pharmaceutical compositions described herein can be administered to a patient using any dosing schedule which achieves the desired therapeutic, prophylactic and/or diagnostic levels for the desired period of time. For example, microparticles or a pharmaceutical composition can be administered and the patient monitored until levels of the bisphosphonate being delivered return to baseline. Following a return to baseline, the microparticles or pharmaceutical composition can be administered again. Alternatively, the subsequent administration of the microparticles or pharmaceutical composition can occur prior to achieving baseline levels in the patient. In one embodiment, the burst of the bisphosphonate is decreased in vivo upon administration to a patient. In one embodiment, the in vivo release of the bisphosphonate is sustained.

The microparticles and pharmaceutical compositions described herein can be used for the treatment of diseases, for example, associated with bone resorption or joint inflammation. In some embodiments, the microparticles and pharmaceutical compositions can be used in treatments for hypercalcemia (e.g., hypercalcemia of malignancy), rheumatoid arthritis, osteoporosis (e.g., menopausal, senile, disease induced and drug induced), Paget's disease of bone (i.e., osteitis deformans) or other bone diseases or conditions such as those characterized by bone resorption.

Exemplification

The invention will now be further and specifically described by the following examples which are not intended to be limiting.

Materials

The following polymers were used in the experiments described infra:
MEDISORB® 5050 DL PLG 4A (hereinafter “5050 4A”), a poly(d,l-lactide-co-glycolide) polymer having 50 mol % d,l-lactide, 50 mol % glycolide, an acid end group, and an inherent viscosity (IV), measured in chloroform at 25° C., of about 0.38 to about 0.48 dL/g.
MEDISORB® 5050 DL PLG 5A (hereinafter “5050 5A”), a poly(d,l-lactide-co-glycolide) polymer having 50 mol % d,l-lactide, 50 mol % glycolide, an acid end group, and an IV of about 0.66 to about 0.80 dL/g.
MEDISORB® 5050 DL PLG HIGH IV (hereinafter “5050 HIGH IV”), a poly(d,l-lactide-co-glycolide) polymer having 50 mol % d,l-lactide, 50 mol % glycolide, a lauryl ester end group, and an IV of about 0.66 to about 0.80 dL/g.
MEDISORB® 7525 DL PLG HIGH IV (hereinafter “7525 HIGH IV”), a poly(d,l-lactide-co-glycolide) polymer having 75 mol % d,l-lactide, 25 mol % glycolide, a lauryl ester end group, and an IV of about 0.75 to about 0.9 dL/g.
MEDISORB® 8515 DL PLG 6A (hereinafter “8515 6A”), a poly(d,l-lactide-co-glycolide) polymer having 85 mol % d,l-lactide, 15 mol % glycolide, an acid end group, and an IV of about 0.59 dL/g.
MEDISORB® 100 DL 4M (hereinafter “100 4M”), a poly(d,l-lactide) polymer having a methyl ester end group, and an IV of about 0.48 dL/g.
MEDISORB® 6535 DL PLG LOW IV (hereinafter “6535 LOW IV”), a poly(d,l-lactide-co-glycolide) polymer having 65 mol % d,l-lactide, 35 mol % glycolide, a lauryl ester end group, and an IV of about 0.50 to about 0.65 dL/g.
MEDISORB® 6535 DL PLG HIGH IV (hereinafter “6535 HIGH IV”), a poly(d,l-lactide-co-glycolide) polymer having 65 mol % d,l-lactide, 35 mol % glycolide, a lauryl ester end group, and an IV of about 0.66 to about 0.80 dL/g.
MEDISORB® 7525 DL PLG LOW IV (hereinafter “7525 LOW IV”),a poly(d,l-lactide-co-glycolide) polymer having 75 mol % d,l-lactide, 25 mol % glycolide, a lauryl ester end group, and an IV of about 0.50 to about 0.65 dL/g.

Each of the above-described polymers are available from Lakeshore Biomaterials, Inc. (Birmingham, Ala.)

Risedronate sodium hydrate, (1-hydroxy-2-(3-pyridinyl)ethylidene) bis(phosphonic acid) monosodium salt, described in U.S. Pat. No. 6,410,520 issued to Cazer, et al., on Jun. 25, 2002, incorporated herein in its entirety, and also referred to in the Exemplification as risedronate sodium bulk drug substance (BDS), was obtained from Procter & Gamble Pharmaceuticals (Cincinnati, Ohio). Alternatively, risedronate sodium can be made using the methods described in U.S. Pat. No. 5,583,122, cited supra.

Experimental Methods

Microparticle Yield

Microparticles were harvested and sieved through a 150 micron screen prior to in vitro and in vivo experiments. The weight of sieved microparticles was divided by the batch starting weight to calculate yield.

In Vitro Release of Risedronate Sodium and Alendronate Sodium

In vitro studies were performed to evaluate the release of risedronate sodium or alendronate sodium from microparticles. Release of risedronate sodium or alendronate sodium from microparticles into a buffer solution was monitored at 1, 4, and 24 hours and weekly thereafter. Cumulative release was calculated as a percentage of the total agent present in a given microparticle batch.

10 milligrams of microparticles and 1 mL of release buffer composition were used for each sample and duplicate samples from each batch were tested. The release buffer composition was Phosphate Buffered Saline (PBS) with 0.02 weight percent polysorbate 20 and 0.02 weight percent sodium azide at pH 7.4. The buffer was completely removed from the microparticles at each timepoint and was thereafter replaced with fresh release buffer composition. The samples were incubated at 37° C.

Risedronate sodium samples were analyzed using isocratic anion exchange High Performance Liquid Chromatography (HPLC) with ultraviolet (UV) detection at 263 nanometers (nm). Alendronate sodium samples were analyzed by a ninhydrin method, whereby alendronate was reacted with ninhydrin to form a colored derivative. The derivative was then transferred to a microtiter plate and optical density was determined. The quantity of alendronate sodium present was determined relative to a standard curve.

In Vivo Release of Risedronate Sodium in Rats

In vivo studies were preformed to evaluate the pharmacokinetic profile of risedronate sodium in rats following administration of a single subcutaneous dose of sieved microparticles.

Male Sprague-Dawley rats (400±50 grams) were obtained from Charles River Laboratories, Inc. (Wilmington, Mass.). Animals were divided into 6 test groups. Each group contained 3 rats.

Each animal was injected subcutaneously once with nominal 200 milligrams of the microparticles. Specifically, the animals were injected subcutaneously into the interscapular region after anesthesia with halothane. The injection vehicle was 3% carboxymethylcellulose (‘CMC’) (low viscosity) and 0.1% TWEEN® 20 (i.e., polyoxyethylene sorbitan monolaureate, TWEEN® is a trademark of ICI Americas, Inc.) in 0.9% aqueous sodium chloride prepared by Baxter Pharmaceutical Solutions (Bloomington, Ind.). Each test subject received a normalized dose of microparticles having about 20 milligrams risedronate sodium per kilogram of body mass. In each case, the administered dose was normalized based on the measured risedronate sodium load in the microparticles as determined by nitrogen analysis, described infra. For example, each animal received a dose comprising approximately 200 milligrams of microparticles containing about 6 to about 10 milligrams of risedronate sodium (about 3 to about 5 weight percent drug load) in a vehicle volume of 1 milliliter.

Blood samples were collected via a lateral tail vein after anesthesia with halothane. Blood samples were collected at predose and at 0.25, 0.5, 1, 2, 4, 8, and 24 hours and then at 2, 4, 7, 14, 21, 28, 35, 42, 49, 56, 63, 70, 77, 84, and 91 days after injection. The lower limit of quantification (“LLOQ”) shown in the figures was based on the average background of about 100 blank blood serum samples.

Particle Size Determination

Microparticle particle size was measured using a Coulter LS Particle Size Analyzer (Model 130, Beckman Coulter, Inc. Fullerton, Calif.). The fluid used was water with dispersant and approximately 45 to 80 milligrams of microparticles were used per analysis. The optical model (with the settings: Fluid reference index, real=1.33; Sample refractive index, real=1.59; and Sample refractive index, imaginary=0) was used to calculate geometric (volume) size statistics.

Bioburden

Microparticles were suspended in fluid thioglycolate media. The suspension was plated onto blood agar and incubated at 28-34° C. Colony forming units were read at least three days after start of incubation.

Load of Risedronate Sodium or Alendronate Sodium in Microparticles

Batch nitrogen content was determined using elemental analysis. The nitrogen result was then used to calculate risedronate sodium or alendronate sodium load in the microparticles by dividing the nitrogen in the microparticles (grams nitrogen/grams of microparticles) by the nitrogen in risedronate sodium or alendronate sodium (grams nitrogen/grams of agent).

An alternative method was sometimes performed for analysis of risedronate sodium content of the microparticles. The method involved dissolution of the microparticles followed by HPLC analysis. 10 mg microparticles were weighed into 2 mL eppendorf tubes in duplicate or triplicate. 200 microliters of an internal standard was added to the tubes, followed by 800 microliters of 1N NaOH. The tubes were rocked overnight at room temperature until complete dissolution of microparticles was achieved. The samples were then diluted in PBS and then analyzed by isocratic anion exchange High Performance Liquid Chromatography (HPLC) with ultraviolet (UV) detection at 263 nanometers (nm).

### EXAMPLE 1

The following example describes the formation of a 5 gram batch of microparticles that include risedronate sodium and a biocompatible polymer using a water-in-oil-in-water (W/O/W) emulsion process.

2.5 milliliters (mL) of reverse osmosis deionized (RODI) water was added to a 20 mL glass scintillation vial containing 290 milligrams (mg) of risedronate sodium bulk drug substance (BDS). The vial was then placed in an 80° C. water bath. The BDS was dissolved by swirling while the temperature was maintained at 80° C., thereby forming an aqueous solution of risedronate at a concentration of about 100 milligrams risedronate sodium/milliliter solution (mg/mL).

4.75 grams (g) of a MEDISORB® polymer were mixed with 21.6 to 34.8 g of ethyl acetate, thereby forming a polymer/ethyl acetate combination with about 12 to about 18 weight percent polymer. The polymer/ethyl acetate combination was poured into a stainless steel funnel with a valve on the bottom. A sonication microtip probe (Model No. CV17; Sonics and Materials, Inc., Danbury, Conn.) was placed about 1 centimeter (cm) below the surface of the polymer/ethyl acetate combination. 2.5 mL of the 80° C. aqueous solution of risedronate was drawn into a sterile 3 mL syringe with a 1.5 in (about 3.8 cm), 18 gauge needle. The sonication was turned on at 40% amplitude. The 80° C. aqueous solution of risedronate was then injected near the microtip probe over an injection time of 14 sec+/−5 sec. Sonication of the resulting mixture was continued for about 1 minute after the end of injection. Thus, a primary, or inner, emulsion (W/O) was formed.

A 0.25 in (about 0.64 cm) outside diameter, 34 element, static mixer constructed of 316 stainless steel (Model No. 04669-60; Cole-Parmer Instrument Co., Vernon Hills, Ill.) was primed for about 5 seconds by pumping a polyvinyl alcohol (PVA) solution, containing 1 weight percent PVA with 6.5 weight percent ethyl acetate, through a T-junction and into the mixer at about 700 mL/min. Following priming, the flow rate of the PVA solution was maintained at about 700 mL/min. The primary emulsion was then pumped through the other branch of the T-junction at a flow rate of 70 mL/min to combine with the PVA solution stream. The combined streams were directed into the static mixer. The primary emulsion was shunted into the PVA solution for about 30+/−5 seconds. Five seconds after the addition of the primary emulsion, the PVA solution stream was turned off.

The static mixer outlet was joined to a dip-tube which emptied into a quench tank. As the mixture of the primary emulsion and PVA solution left the static mixer, the combined stream flowed into the quench tank. The quench tank was initially charged with 3.5 liters of RODI water at room temperature. The quench tank was equipped with an impeller stirring at about 300 to about 400 rpm. Following addition of the combined primary emulsion and PVA solution streams, the quench tank was stirred for 30 minutes.

After 30 minutes of residence in the quench tank, a valve on the bottom of the quench tank was opened and the contents of the tank were directed into a 25 micron stainless steel sieve (diameter of 21 cm) to collect the microparticles. The microparticles were washed in the sieve with a continuous flow of RODI water for about 3 to 5 minutes. The microparticles were transferred to a sterile glass dish with a diameter of about 3 to 5 cm and the dish was covered with a Kimwipe. The glass dish was placed in a freezer at −80° C. for at least about 30 minutes. The glass dish was then placed in a pre-chilled lyophilizer (Model No. TD-2C-MP; FTS Systems, Inc., Stone Ridge, N.Y.) with a shelf temperature of about −40° C. The following lyophilization program was then performed: started at −40° C., 150 millitorr (mT); ramped 2.5° C./min to −10° C.; held 5 hours at 300 mT; ramped 2.5° C./min to 30° C.; and held for 2 days at 300 mT. The microparticles were then poured into a sterilized 150 micron stainless steel sieve (6 cm diameter) and the microparticles were sieved by banging and breaking microparticle masses with a spatula. The fraction of the microparticles that passed through the sieve were collected as the microparticle product.

### EXAMPLE 2

The following example describes the formation of a 5 gram batch of microparticles that include risedronate sodium and a biocompatible polymer using a solid-in-oil-in-water (S/O/W) emulsion process.

4.75 g of a MEDISORB® polymer was mixed with 21.6 to 34.6 g of ethyl acetate, thereby forming a polymer/ethyl acetate combination. 290 mg of risedronate sodium BDS (milled using a 1 inch model Standard Micron-Master Mill) were then dispersed in the polymer/ethyl acetate combination using a Sonics and Materials, Inc. Model No. CV17 sonication probe, thereby forming a suspension, i.e., a solid-in-oil (S/O) suspension, that includes a dispersed phase containing the bisphosphonate compound and a continuous phase of a polymer combination containing a biocompatible polymer and a solvent of the polymer.

A 0.25 in (about 0.64 cm) outside diameter, 34 element, static mixer constructed of 316 stainless steel (Model No. 04669-60; Cole-Parmer Instrument Co., Vernon Hills, Ill.) was primed for about 5 seconds by pumping a polyvinyl alcohol (PVA) solution, containing 1 weight percent PVA with 6.5 weight percent ethyl acetate, through a T-junction and into the mixer at about 700 mL/min. Following priming, the flow rate of the PVA solution was maintained at about 700 mL/min. The S/O suspension was then pumped through the other branch of the T-junction at a flow rate of 70 mL/min to combine with the PVA solution stream. The combined streams were directed into the static mixer. The S/O suspension was shunted into the PVA solution for about 30+/−5 seconds. Five seconds after the addition of the S/O suspension, the PVA solution stream was turned off.

The static mixer outlet was joined to a dip-tube which emptied into a quench tank. As the mixture of the S/O suspension and PVA solution left the static mixer, the combined stream flowed into the quench tank. The quench tank was initially charged with 3.5 liters of RODI water at room temperature. The quench tank was equipped with an impeller stirring at about 300 to about 400 rpm. Following addition of the combined S/O suspension and PVA solution streams, the quench tank was stirred for 30 minutes.

After about 30 to 60 minutes of residence in the quench tank, the microparticles were filtered, washed, lyophilized, and sieved as described in Example 1.

### EXAMPLE 3

The following example describes the formation of a 5 gram batch of microparticles that include risedronate sodium and a biocompatible polymer using a water-in-oil-in-oil (W/O/O) emulsion process.

290 mg of risedronate sodium BDS were mixed with 2.5 mL of water at 70° C., thereby forming an aqueous solution of risedronate at a concentration of about 100 milligrams risedronate/milliliter solution (mg/mL). 4.75 g of a MEDISORB® polymer was mixed with about 32 to about 36 mL of methylene chloride, thereby forming a polymer/methylene chloride combination. The aqueous solution of risedronate sodium was then mixed with the polymer/methylene chloride combination using a Sonics and Materials, Inc. Model No. CV17 sonication probe, thereby forming an inner water-in-oil (W/O) emulsion.

The inner water-in-oil emulsion was then added to a vessel and an equivalent volume of silicon oil (DOW CORNING® Medical Fluid, 350 CST; Dow Corning Corp., Midland, Mich.; DOW CORNING® is a trademark of Dow Corning Corp.) (e.g., about 32 to about 36 mL) was pumped into the vessel in a total of about 3 minutes with mixing at about 1,100 rpm to emulsify and thereby form a water-in-oil-in-oil (W/O/O) emulsion. The emulsion was then added to 2,800 mL of a 50/50 (by volume) heptane/ethanol mixture with mixing for 30 minutes. The resulting heptane/ethanol decanted off and 500 mL of heptane was added to the emulsion in a quench tank and the contents were mixed. After about 1 hour of residence in the quench tank at about 5° C. to about 15° C., a valve on the bottom of the quench tank was opened and the contents of the tank were directed into a 25 micron stainless steel sieve (diameter of 21 cm) to collect the microparticles. Residual heptane, ethanol and methylene chloride were removed from the microparticles by drying the microparticles using a continuous nitrogen stream for about 10 to about 24 hours at about 0° C., then for about 24 hours at about 25° C., and, finally, for at least two days at about 37° C.

### EXAMPLE 4

The following example describes the formation of a 5 gram batch of microparticles that include risedronate sodium and a biocompatible polymer using a solid-in-oil-in-oil (S/O/O) emulsion process.

4.75 g of a MEDISORB® polymer was mixed with 36.1 to about 67.9 mL of methylene chloride, thereby forming a polymer/methylene chloride combination. 250 mg of lyophilized risedronate sodium, produced by forming an aqueous solution of the BDS, spraying the solution, and lyophilizing the sprayed product, as generally described supra, were then dispersed in the polymer/methylene chloride combination using a Sonics and Materials, Inc. Model No. CV17 sonication probe, thereby forming a suspension, i.e., a solid-in-oil (S/O) suspension.

The S/O suspension was then added to a vessel and an equivalent volume of silicon oil (DOW CORNING® Medical Fluid, 350 CST; Dow Corning Corp., Midland, Mich.; DOW CORNING® is a trademark of Dow Corning Corp.) (e.g., about 32 to about 36 mL) was pumped into the vessel in a total of about 3 minutes with mixing at about 1,100 rpm to emulsify and thereby form a solid-in-oil-in-oil (S/O/O) emulsion. The emulsion was then added to 2,800 mL of a 50/50 (by volume) heptane/ethanol mixture with mixing for 30 minutes. The heptane/ethanol was decanted off and 500 mL of heptane was added to the emulsion in a quench tank and the contents were mixed. After about 1 hour of residence in the quench tank at about 5° C. to about 15° C., a valve on the bottom of the quench tank was opened and the contents of the tank were directed into a 25 micron stainless steel sieve (diameter of 21 cm) to collect the microparticles. Residual heptane, ethanol and methylene chloride were removed from the microparticles by drying the microparticles using a continuous nitrogen stream for about 10 to about 24 hours at about 0° C., then for about 24 hours at about 25° C., and, finally, for at least two days at about 37° C.

### EXAMPLE 5

Table 1 shows several microparticle formulations that were generally formed as described in Examples 1-4, above. The microparticles of each formulation were made using a target risedronate sodium load of about 5 weight percent and a target polymer content of about 95 weight percent (both based on final microparticle weight).

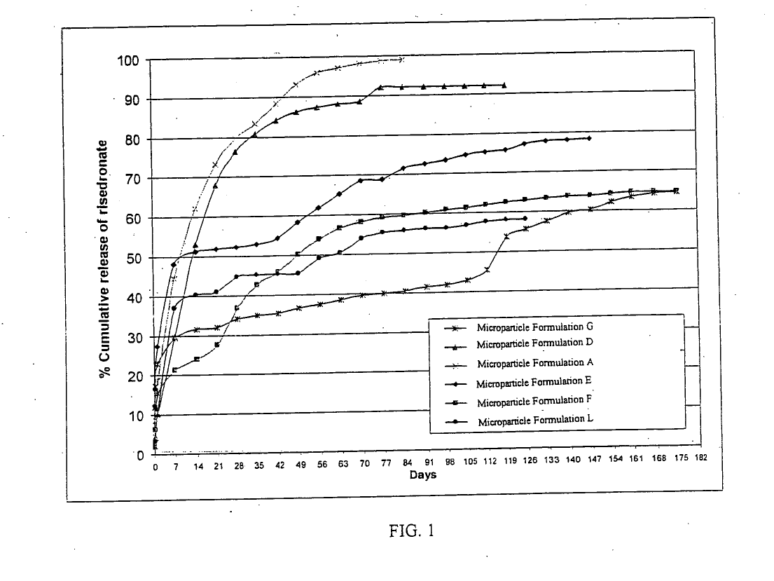

In vitro release was used to select formulations with low initial release for subsequent pharmacokinetic (PK) studies. An initial release phase was characterized by measuring drug released at 1, 4 and 24 hours. A 24 hour percent cumulative release, shown in Table 2, was calculated by summing the amount of drug released at 1, 4 and 24 hours relative to the total initial drug load in the microparticles as determined by nitrogen analysis. FIG. 1 demonstrates that the in vitro release rate of risedronate sodium was dependent on polymer type. In general, microparticles containing polymers with higher lactide content had reduced rates of risedronate release. Polymer end group and molecular weight also likely influenced rates of risedronate release.

TABLE 1
Microparticle Formulations

| | | | | Polymer |
|---|---|---|---|---|
| | | | | Concentra- |
| | | | | tion |
| | | | Risedronate | in |
| | | | Sodium | Polymer/ |
| Microparticle | Emulsion | | Content | Solvent |
| Formulation | Process | Polymer | (wt %) | Combination |
| A | W/O/O | 5050 4A | 4.5% | 9 wt % |
| B | W/O/O | 5050 5A | 4.1% | 9 wt % |
| C | W/O/O | 5050 HIGH IV | 4.3% | 10 wt % |
| D | S/O/O | 5050 4A | 4.1% | 9 wt % |
| E | S/O/O | 7525 HIGH IV | 3.9% | 5 wt % |
| F | S/O/O | 8515 6A | 4.1% | 7 wt % |
| G | S/O/O | 100 4M | 4.3% | 7 wt % |
| H | W/O/W | 6535 LOW IV | 5.0% | 15 wt % |
| I | W/O/W | 6535 LOW IV | 2.2% | 15 wt % |
| J | W/O/W | 6535 HIGH IV | 2.8% | 15 wt % |
| K | W/O/W | 6535 HIGH IV | 2.8% | 18 wt % |
| L* | W/O/W | 7525 LOW IV | 3.8% | 19 wt % |
| M | W/O/W | 7525 LOW IV | 3.6% | 15 wt % |
| N | W/O/W | 7525 LOW IV | 4.1% | 15 wt % |
| O** | W/O/W | 7525 LOW IV | 1.1% | 10 wt % |
| P** | S/O/W | 7525 LOW IV | 1.1% | 10 wt % |
| Q | W/O/W | 7525 HIGH IV | 3.6% | 12 wt % |
| R | W/O/W | 8515 6A | 4.9% | 18 wt % |
| S | W/O/W | 100 4M | 3.3% | 18 wt % |
| *These microparticles were produced as described supra except the polymer/solvent mixture was heated to 65° C. prior to formation of the inner emulsion, the aqueous risedronate solution contained 150 mg | | | | |
| # risedronate sodium/mL, and a 10° C., pH 3 citrate quench was used. | | | | |

**These microparticles were produced as described supra except the polymer/solvent mixture was heated to 65° C. prior to formation of the inner emulsion.

TABLE 2
Pharmacokinetic Data for Risedronate Sodium-Containing Microparticles

| | 24-hour In Vitro | | |
|---|---|---|---|
| Microparticle | Release | C_{MAX} | AUC_{0-1 DAY} |
| Formulation | (Cumulative %) | (ng/mL) | (ng day/mL) |
| A | 19% | 4590 ± 920 | 1770 ± 290 |
| B | 13% | 1110 ± 380 | 960 ± 210 |
| C | 5% | 960 ± 180 | 480 ± 80 |
| D | 10% | 1970 ± 750 | 860 ± 110 |
| E | 27% | 4800 ± 2220 | 2160 ± 430 |
| F | 15% | 1710 ± 200 | 800 ± 150 |
| G | 23% | 5840 ± 580 | 1590 ± 410 |
| H | 14% | 2330 ± 390 | 750 ± 80 |
| K | 10% | 2780 ± 550 | 1030 ± 130 |
| M | 4% | 2850 ± 1380 | 600 ± 250 |
| N | 5% | 1180 ± 230 | 300 ± 20 |
| P | 8% | 370 ± 30 | 470 ± 130 |
| Q | 2% | 663 ± 110 | 300 ± 60 |
| R | 3% | 464 ± 50 | 150 ± 40 |
| S | 3% | 1160 ± 180 | 360 ± 50 |

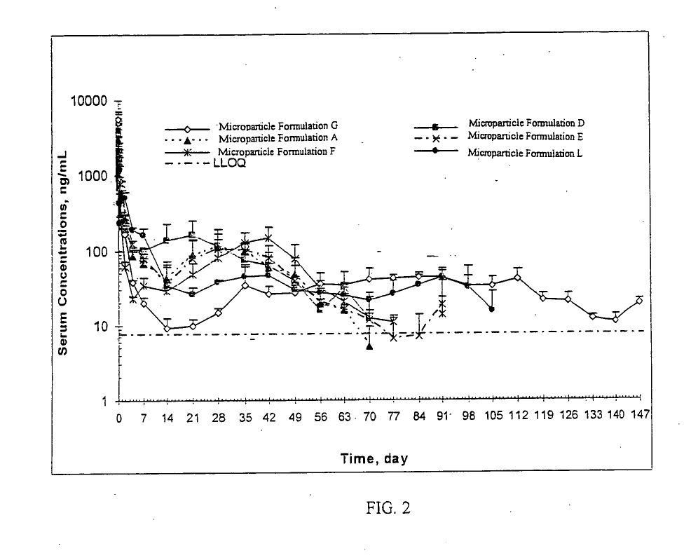

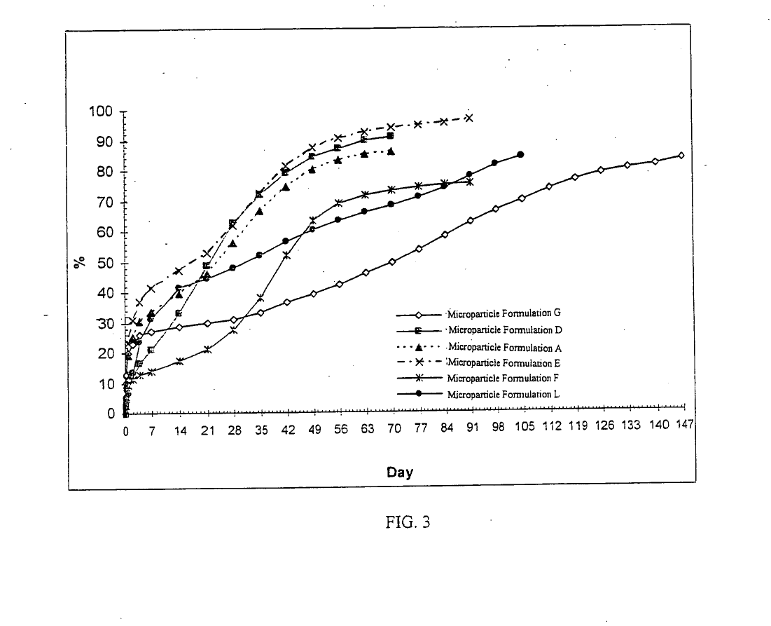

FIGS. 2 and 3 show corresponding in vivo PK serum and cumulative Area Under the Curve (AUC) profiles for selected microparticle formulations. The in vivo PK profiles were roughly similar to in vitro release profiles in magnitude of initial release, general profile shape and duration. In the sustained release phase (beyond 7 days) in vivo, the microparticle formulations with higher lactide content had reduced rates of drug release (lower serum levels) and extended duration.

Microparticle formulations containing MEDISORB® 5050 4A and 7525 HIGH IV released the drug at the fastest rate and maintained significant serum levels of risedronate sodium out through 2 months. The formulation containing MEDISORB® 8515 DL 6A had measurable levels of risedronate approaching 3 months. The MEDISORB® 7525 LOW IV formulation had measurable levels beyond 3 months and the MEDISORB® 100 4M formulation had measurable levels at 5 months. In all cases, greater than 75 weight percent of the risedronate sodium was accounted for in vivo as compared to a subcutaneous comparator and shown in FIG. 3.

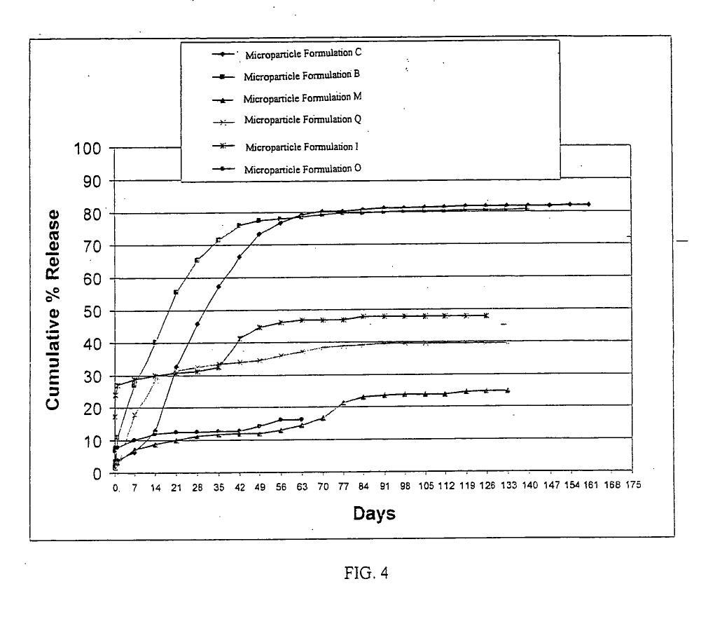

FIG. 4 shows in vitro release for five formulations. Formulations C, M, and Q all had risedronate sodium loading of greater than 3 weight percent and 24 hour in vitro releases of 5 percent of less. The in vitro profiles of W/O/W process formulations had somewhat flatter profiles and accounted for less drug than W/O/O process formulations. The loss of accounted for drug can be explained, in part, from mass loss of microparticles during in vitro assay buffer transfers. In contrast to W/O/O process formulations, the W/O/W process formulations tended to float and stick to pipette tips and tube surfaces.

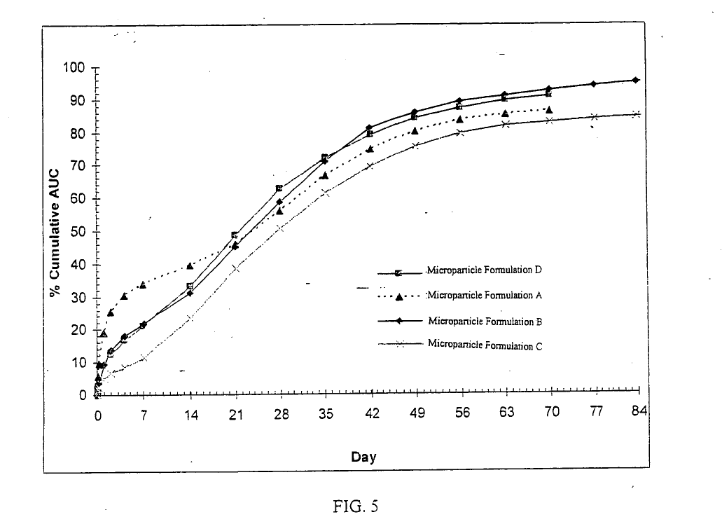

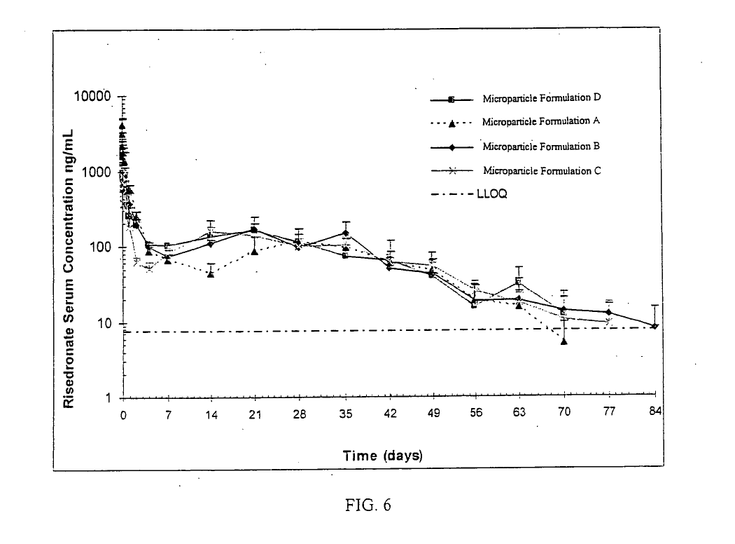

FIGS. 5 and 6 demonstrate how polymer molecular weight and end group appear to have influenced the in vivo PK profile. For the W/O/O process formulations (Formulations A, B and C), higher polymer molecular weight and a lauryl end group resulted in improved microparticle PK performance. Formulation C, containing a polymer with a lauryl ester end group, had the lowest in vivo day 1 AUC and had a release the closest to approximating a zero order release. All four formulations had similar duration, with measurable serum levels of risedronate for at least 10 weeks. FIG. 6 shows that the microparticles containing higher molecular weight polymers such as MEDISORB® 5050 5A and 5050 HIGH IV may have extended duration by about one to two weeks.

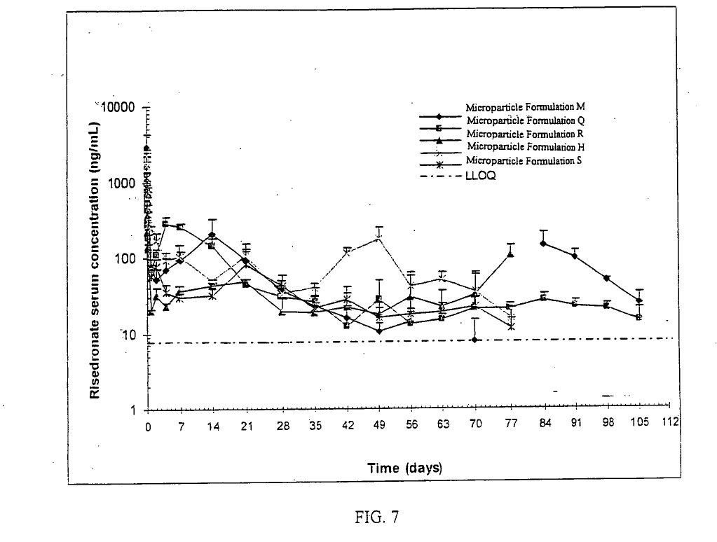

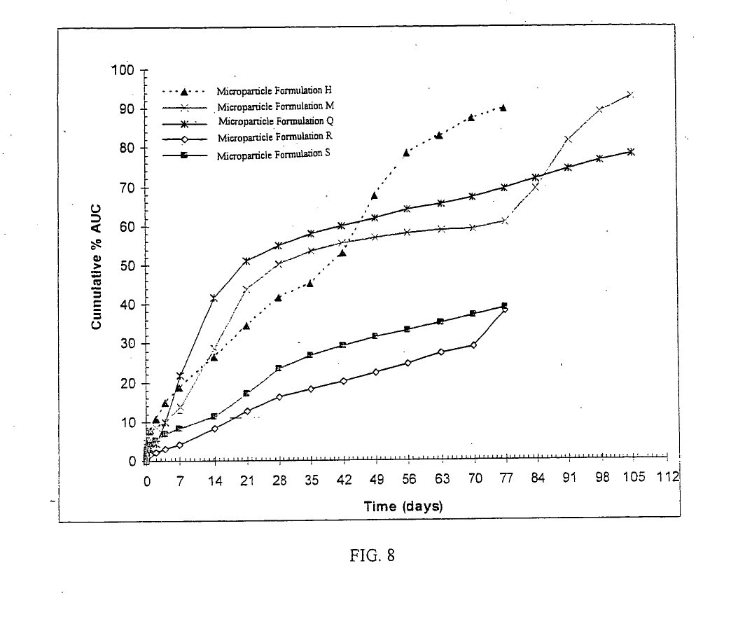

FIGS. 7 and 8 show corresponding in vivo PK serum and cumulative area under the curve (AUC) profiles for selected microparticles formed using the W/O/W emulsion process. Table 2 summarizes in vitro 24 hour percent cumulative release, maximum in vivo blood serum concentration (Cₘₐₓ), and in vivo area under the curve up to day 1 (AUC_{0-1 DAY}) for each of the microparticle formulations of Table 1. In general, microparticles produced using the using the W/O/W and S/O/W emulsion processes had lower in vitro 24 hour percent cumulative release and lower AUC_{0-1 DAY} than microparticles produced using the W/O/O and S/O/O emulsion processes. Among microparticles produced using the using the W/O/W and S/O/W emulsion processes, microparticles made using polymers having higher lactide content had typically lower rates of release of risedronate and longer duration.

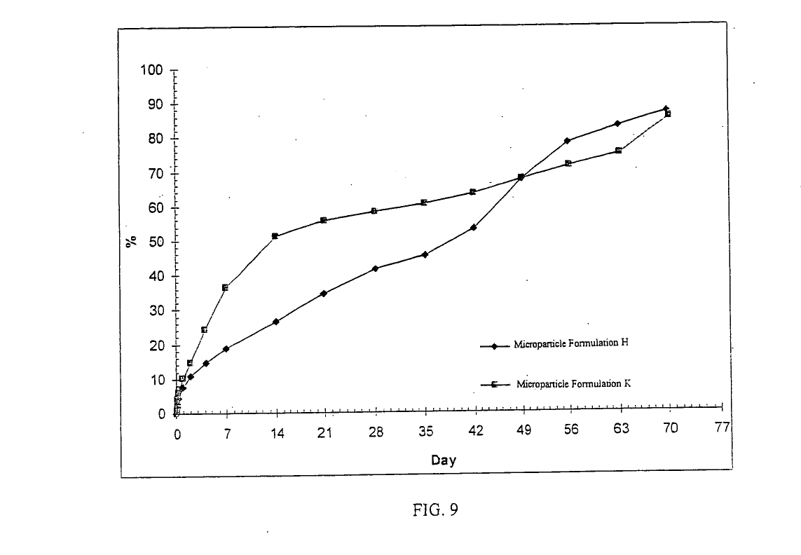

FIGS. 8 and 9 demonstrate the effect of molecular weight with lauryl end group polymers on cumulative AUC profiles. FIG. 8 shows that Microparticle Formulation Q, (containing 7525 HIGH IV) had a similar profile to Formulation M (containing 7525 LOW IV) until day 77, after which Formulation M maintained higher serum levels of the risedronate. (Note that 7525 HIGH IV has a higher molecular weight than 7525 LOW IV.)

FIG. 9 demonstrates that the microparticles having a higher molecular weight polymer (Formulation K) apparently releases risedronate faster during the first two weeks than the microparticles that contained a lower molecular weight polymer (Formulation H). Beyond two weeks, the rate of risedronate release slowed for Formulation K and was less than the rate of release of risedronate from Formulation H. Without being held to any particular theory, it is believed that slower release may have been caused by slower degradation of the higher molecular weight polymer and/or by the lower availability of risedronate for release from the microparticles after a higher first two weeks of release. It is also believed that because risedronate release was higher in the first two weeks for the microparticles containing a higher molecular weight polymer, molecular weight may play a more complex role than just control of release degradation and that there may be differences in microparticle morphology linked to polymer molecular weight that can influence early release of risedronate.

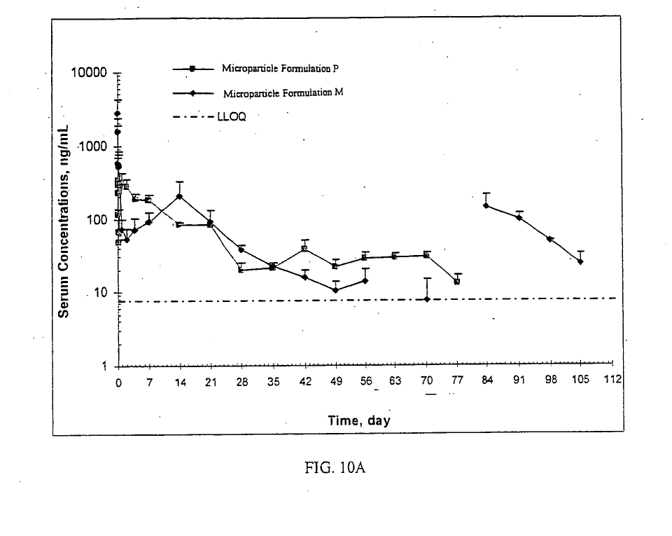

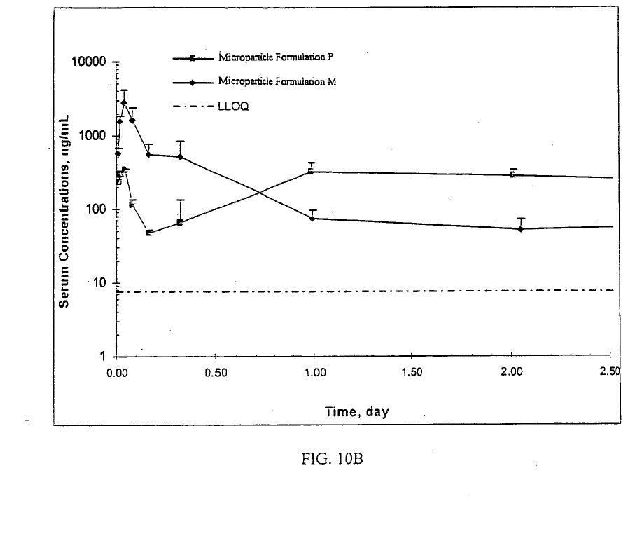

FIGS. 10A and 10B show the effect on rat blood serum concentrations of risedronate post subcutaneous administration of microparticles containing 7525 LOW IV polymer and prepared using the W/O/W emulsion and S/O/W emulsion methods. The microparticles produced using the W/O/W emulsion process (Microparticle Formulation M) had higher C_{MAX} and AUC_{0-1 DAY}. The microparticles produced using the S/O/W emulsion process (Microparticle Formulation P) had a time to maximum risedronate blood serum concentration (T_{MAX}) that occurred at about 16 hours on average versus at about one hour for the microparticles produced using the W/O/W emulsion process. Beyond about 14 days through about 60 days, the blood serum concentrations of risedronate were similar for both formulations.

In addition to the microparticles formed as described supra, microparticles were also produced using a W/O/W emulsion process as generally described in Example 1 wherein MEDISORB® 5050 4A and 5050 HIGH IV polymers were used. Since poly(d,l-lactide-co-glycolide) polymers having 50 mol % d,l-lactide, 50 mol % glycolide are generally not soluble in ethyl acetate, methylene chloride was employed instead. The resulting microparticles exhibited relatively poor incorporation (e.g., a risedronate load of less than about 3 weight percent) or a relatively poor sieve yield (e.g., had a high degree of polymer agglomeration) as compared to other microparticles that were produced as described herein.

### EXAMPLE 6

This example describes formation of several microparticle formulations (5 gram scale Microparticle Formulations H, M, Q, R and S) at a 20 gram scale. Table 3 shows process compositions for 20 gram scale Microparticle Formulations T, U, V, W and X that were produced.

TABLE 3
Process Compositions for 20 Gram Scale Microparticle Production

| Microparticle | | Polymer/Ethyl Acetate Combination: |
|---|---|---|
| Formulation | Polymer | (wt % polymer in ethyl acetate) |
| T | 6535 LOW IV | 15 |
| U | 7525 LOW IV | 15 |
| V | 7525 HIGH IV | 12 |
| W | 8515 6A | 18 |
| X | 100 4M | 18 |

10 milliliters (mL) of reverse osmosis deionized (RODI) water was added to a 20 mL glass scintillation vial containing 1157 milligrams (mg) of risedronate sodium BDS. The vial was then placed in an 80° C. water bath. The BDS was dissolved by swirling while the temperature was maintained at 80° C., thereby forming an aqueous solution of risedronate at a concentration of about 100 milligrams risedronate sodium/milliliter solution (mg/mL).

19 g of a MEDISORB® polymer were mixed with about 86.6 to about 139.3 g of ethyl acetate, thereby forming a polymer/ethyl acetate combination with 12, 15 or 18 weight percent polymer. The polymer/ethyl acetate combination was poured into a stainless steel funnel with a valve on the bottom. A sonication microtip probe (Model No. CV17; Sonics and Materials, Inc., Danbury, Conn.) was placed about 1 centimeter (cm) below the surface of the polymer/ethyl acetate combination. 10 mL of the 80° C. aqueous solution of risedronate was drawn into a sterile 3 mL syringe with a 1.5 in (about 3.8 cm) 18 gauge needle. The sonication was turned on at 40% amplitude. The 80° C. aqueous solution of risedronate was then injected near the microtip probe over an injection time of 14 sec+/−5 sec. Sonication of the resulting mixture was continued for about 4 minutes after the end of injection. Thus, a primary, or inner, emulsion (W/O) was formed.

A 0.25 in (about 0.64 cm) outside diameter, 34 element, static mixer constructed of 316 stainless steel (Model No. 04669-60; Cole-Parmer Instrument Co., Vernon Hills, Ill.) was primed for about 5 seconds by pumping a polyvinyl alcohol (PVA) solution, containing 1 weight percent PVA with 6.5 weight percent ethyl acetate, through a T-junction and into the mixer at about 700 mL/min. Following priming, the flow rate of the PVA solution was maintained at about 700 mL/min. The primary emulsion was then pumped through the other branch of the T-junction at a flow rate of 70 mL/min to combine with the PVA solution stream. The combined streams were directed into the static mixer. The primary emulsion was shunted into the PVA solution for about 30+/−5 seconds. Five seconds after the addition of the primary emulsion, the PVA solution stream was turned off.

The static mixer outlet was joined to a dip-tube which emptied into a quench tank. As the mixture of the primary emulsion and PVA solution left the static mixer, the combined stream flowed into the quench tank. The quench tank was initially charged with 15 liters of RODI water at room temperature. The quench tank was mixed with a stir bar at about 300 to about 400 rpm. Following addition of the combined primary emulsion and PVA solution streams, the quench tank was stirred for 30 minutes.

After 1 hour of residence in the quench tank, a valve on the bottom of the quench tank was opened and the contents of the tank were directed into a 25 micron stainless steel sieve (diameter of 21 cm) to collect the microparticles. The microparticles were washed in the sieve with a continuous flow of RODI water for about 3 to 5 minutes. The microparticles were transferred to a sterile glass dish with a diameter of about 12 cm and the dish was covered with a Kimwipe. The glass dish was placed in a freezer at −80° C. for at least about 30 minutes. The glass dish was then placed in a pre-chilled lyophilizer (Model No. TD-2C-MP; FTS Systems, Inc., Stone Ridge, N.Y.) with a shelf temperature of about −40° C. The following lyophilization program was then performed: started at −40° C., 150 millitorr (mT); ramped 2.5° C./min to −10° C.; held 5 hours at 300 mT; ramped 2.5° C./min to 30° C.; and held for 2 days at 300 mT. The microparticles were then poured into a sterilized 150 micron stainless steel sieve (6 cm diameter) and the microparticles were sieved by banging and breaking microparticle masses with a spatula. The fraction of the microparticles that passed through the sieve were collected as the microparticle product.

During formation of the primary W/O emulsion, differing degrees and sizes of particulates in the emulsion, depending on the polymer type used, were observed. A light microscope revealed that both polymer precipitate and drug crystals as well as water droplets (emulsion) were present in the W/O emulsion. After the W/O emulsion was removed from the sonication vessel, varying amounts of risedronate and polymer deposits were present on the vessel surface. A reduced rate of sonication energy input is thought to have contributed to this condition. This phenomenon appeared less severe when MEDISORB® 7525 LOW IV (Microparticle Formulation U) and MEDISORB® 7525 HIGH IV (Microparticle Formulation V) were used.

Table 4 shows performance characteristics of Microparticle Formulations T, U, V, W and X.

TABLE 4
Twenty Gram Scale Microparticle Performance Characteristics

| Microparticle | Risedronate Sodium Loading | 24 Hour In Vitro Release |
|---|---|---|
| Formulation | (by Nitrogen Analysis) (wt %) | (Cumulative %) |
| T | 3.3% | 9 |
| U | 4.1% | 2 |
| V | 3.3% | 3 |
| W | 3.0% | 18 |
| X | 2.7% | 11 |

Microparticle Formulations T, U and V, containing MEDISORB® 6535 LOW IV, 7525 LOW IV and 7525 HIGH IV, respectively, showed good loading of risedronate sodium and initial in vitro release.

Microparticle Formulation X, containing MEDISORB® 100 DL 4M, had a risedronate sodium loading of only 2.7%. In order to reduce the above-described particulate and precipitate formation, a series of microparticle formulations were made wherein a number of formulation process parameters were varied. Microparticle Formulation X-1 was formed as for Formulation X, described supra, except that the polymer/ethyl acetate combination used had a polymer concentration of 15 weight percent. Microparticle Formulation X-2 was formed as for Formulation X, described supra, except that the aqueous solution of risedronate was formed at 90° C. Microparticle Formulation X-3 was formed as for Formulation X, described supra, except that instead of a sonication probe, an IKA rotor/stator homogenizer, Model No. T25S6 (IKA Works USA, Wilmington, N.C.) operating at maximum speed (24,000 rpm) was used to form the inner W/O emulsion. Microparticle Formulation X-4 was formed as for Formulation X, described supra, except that the temperature of the polymer/ethyl acetate combination was about 60-65° C. Microparticle Formulation X-5 was formed as for Formulation X, described supra, except that 13.3 mL of water was used to form the aqueous solution of risedronate at a temperature of 70° C. and having a concentration of 75 mg/mL. Microparticle Formulation X-6 was formed using a S/O/W process as described supra and using risedronate sodium milled using a Standard Micron-Master Mill, 1 inch model (Jet Pulverizer Co., Moorestown, N.J.) at the maximum speed.

Table 5 shows observations and resulting microparticle characteristics for the various additional microparticle formulations. The inner emulsion relative amount and size of the precipitate was accessed visually and is shown on a relative scale with fewer “+” symbols representing fewer and/or smaller precipitates relative to the control (Microparticle Formulation X).

TABLE 5
Microparticles Produced by Varying Processing Conditions

| | Inner | | |
|---|---|---|---|
| | Emulsion | | |
| | (Relative | | |
| | Amount and | Risedronate Sodium | 24 hour In Vitro |
| Microparticle | Size of | Loading (by Nitrogen | Release |
| Formulation | Precipitate) | Analysis) | (Cumulative %) |
| X (control) | ++++ | 2.7 | 11 |
| X-1 | +++++ | 2.0 | 4 |
| X-2 | +++ | 2.8 | 7 |
| X-3 | + | 4.5 | 10 |
| X-4 | + | 3.9 | 5 |
| X-5 | ++ | 3.8 | 39 |
| X-6 | + | 4.7 | 7 |

Approaches that limited particulate/precipitate formation, while having acceptable risedronate sodium load and 24 hour in vitro release, included those that employed rotor/stator homogenization and wherein the polymer/ethyl acetate solution was heated to 60-65° C. prior to inner emulsion formation (e.g., Microparticle Formulations X-3 and X-4).

Based upon these results, the most effective approaches were applied to Microparticle Formulation W. Microparticle Formulation W-1 was formed as for Formulation W, described supra, except that instead of a sonication probe, an IKA Model No. T25S6 rotor/stator homogenizer (IKA Works USA, Wilmington, N.C.) operating at the maximum speed of 24,000 rpm was used to form the inner W/O emulsion. Microparticle Formulation W-2 was formed as for Formulation W, described supra, except that the temperature of the polymer/ethyl acetate combination was about 60-65° C. Microparticle Formulation W-3 was formed as for Formulation W, described supra, except that 13.3 mL of water was used to form the aqueous solution of risedronate at a temperature of 70° C. and having a concentration of 75 mg/mL.

Table 6 shows observations and resulting microparticle characteristics for the various microparticle formulations. The inner emulsion relative amount and size of the precipitate was accessed visually and is shown on a relative scale with fewer “+” symbols representing fewer and/or smaller precipitates relative to the control (Microparticle Formulation W).

TABLE 6
Microparticles Formed by Varying Processing Conditions

| | | Risedronate | |
|---|---|---|---|
| | Inner Emulsion | Sodium | |
| | (Relative Amount | Loading (by | 24 hour In Vitro |
| Microparticle | and Size of | Nitrogen | Release |
| Formulation | Precipitate) | Analysis) | (Cumulative %) |
| W (control) | +++ | 3.0 | 18 |
| W-1 | +++ | 4.0 | 15 |
| W-2 | + | 3.6 | 4 |
| W-3 | + | 2.9 | 33 |

The process variation used for Microparticle Formulation W-2 (i.e., the process that included heating the polymer/ethyl acetate mixture to about 60-65° C. prior to forming the inner emulsion) limited particulate/precipitate formation and yielded microparticles with acceptable risedronate load and initial in vitro release.

Table 7 shows in vitro and in vivo test results for Microparticle Formulations T, U, V, W-2 and X-4.

TABLE 7
Twenty Gram Scale Microparticle Performance

| | 24-hour In Vitro | | |
|---|---|---|---|
| Microparticle | Release | C_{MAX} | AUC_{0-1 DAY} |
| Formulation | (Cumulative %) | (ng/mL) | (ng day/mL) |
| T | 9% | 4150 ± 950 | 1060 ± 140 |
| U | 2% | 860 ± 170 | 240 ± 50 |
| V | 3% | 910 ± 110 | 430 ± 50 |
| W-2 | 4% | 2310 ± 400 | 630 ± 50 |
| X-4 | 5% | 2300 ± 550 | 390 ± 50 |

Microparticle Formulations T, U, V, W-2 and X-4 each had greater than 3 weight percent risedronate sodium load, less than 10% 24 hour in vitro release, and low bioburden and were considered acceptable for further study.

In vivo performance of Microparticle Formulations T, U, V, W-2 and X-4 (each made at the 20 gram scale) was compared to similar batches previously made at the 5 gram scale (i.e., Microparticle Formulations T, M and N, Q, R, and S, respectively). Note that process conditions for Formulations W-2 and X-4 were changed from those of Formulations W and X upon scale-up (i.e., the polymer/ethyl acetate combination temperature was increased at the 20 gram scale).

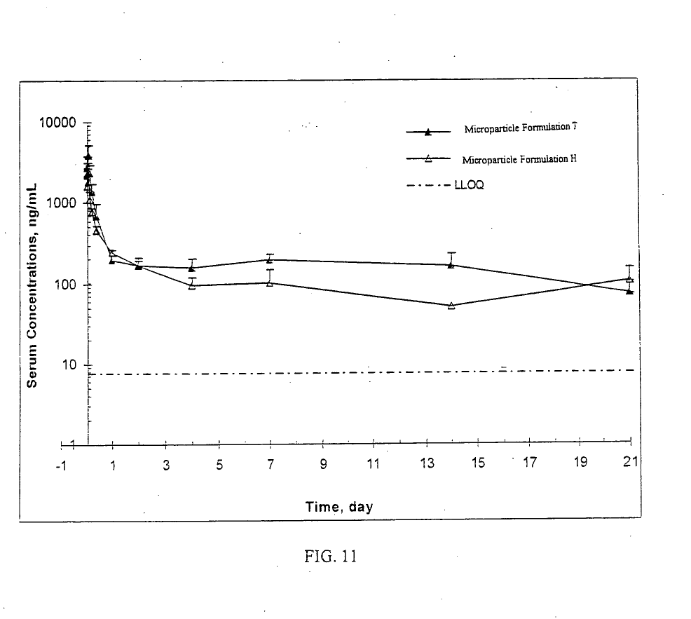

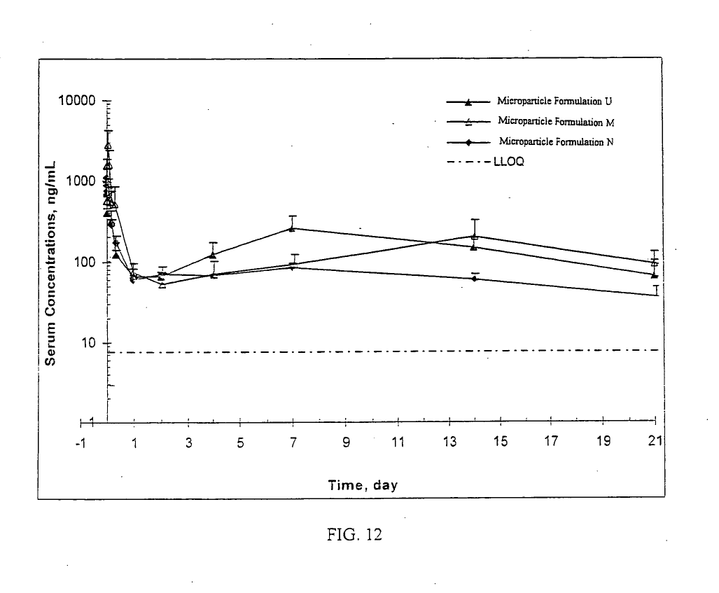

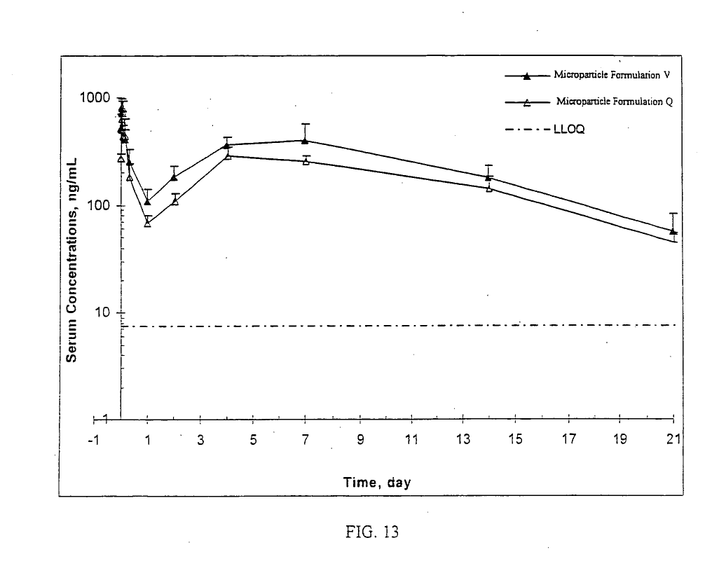

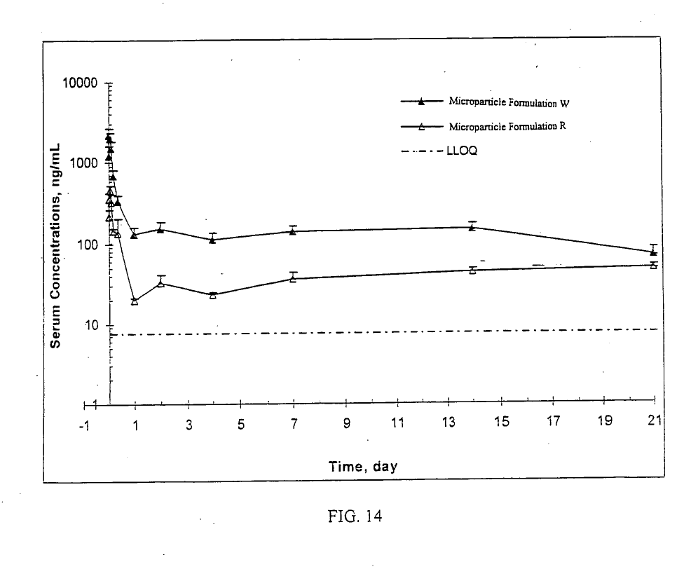

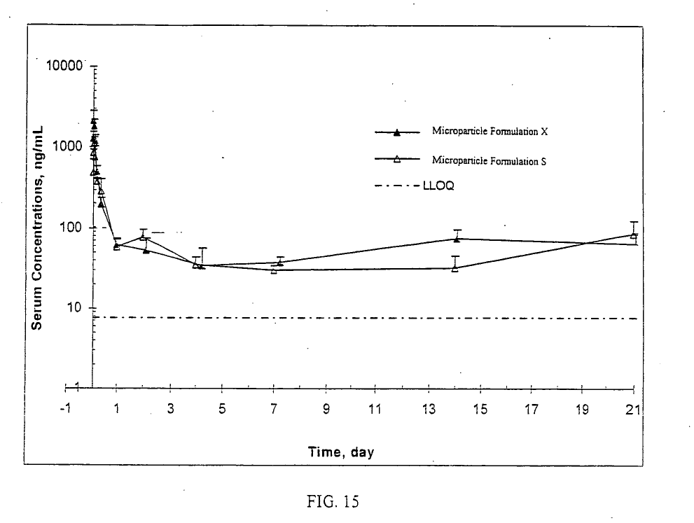

FIGS. 11 through 15 demonstrate that, through 14 days, the PK profiles for similar formulations were comparable when produced at the 5 gram versus the 20 gram scale. Exceptions were Formulations W and W-4, for which the 20 gram batches exhibited higher levels of risedronate sodium relative to the 5 gram batch through 14 days. Each of Microparticle Formulations T, M, N, Q, R, S, T, U, V, W-2 and X-4 demonstrated reproducible sustained release for the 20 gram and 5 gram batches in rat and no unacceptable local tolerability effects were observed upon administration of the microparticle formulations to the animals.

The local tolerability effects of subcutaneous doses of several microparticle formulations (Microparticle Formulations A, E, G, U, V, W-2, D, S, N, X-4, and T) and comparator placebo microparticles were assessed in rat. For a nominal 100 mg microparticle dose containing about 5 mg of risedronate sodium, all microparticle formulations tested were considered well-tolerated and (based on tolerability criteria) suitable for clinical testing. For example, at Day 14, histological observations of the injection site generally showed mild fibrous encapsulation of residual microparticles, mild to minimal foreign body and/or mixed cell inflammation and no or minimal purulent or inflammatory exudate. Comparator placebo microparticle formulations of the same polymer type, dose mass and volume generally showed mild to minimal fibrous encapsulation, mild foreign body inflammation and no/minimal purulent or inflammatory exudate. The diameter of the injection sites was measured externally, and averages for risedronate sodium groups ranged from 1.1 cm to 2.3 cm for the period Day 1 through Day 28 post-injection. Placebo microparticle injection site diameter group averages were slightly smaller, ranging from an average of 0.8 cm to 1.7 cm for the same time period.

In general, low initial release (e.g., less than 5% cumulative in vitro release at 24 hours) microparticle formulations (e.g., Microparticle Formulations U, V, and W-2) showed a milder histological response at Day 14 and smaller injection site diameters for the period 1 day through 28 days post-injection, compared to high initial release formulations (e.g., at least 19% cumulative in vitro release at 24 hours, e.g., Microparticle Formulations A, E, and G). The histological and injection site observations from the low initial release formulations were generally more consistent with observations from placebo microparticles than from the high initial release formulations. Injection site diameter measurements for low initial release formulations averaged 1.3 to 1.6 cm for the period 1 day through 28 days post-injection, similar to measurements from comparator placebo formulations, which averaged 1.2 to 1.7 cm for the same time period. High burst formulations' injection site diameters were larger than corresponding placebo microparticles, averaging 2.2 to 2.3 cm for the period 1 day through 28 days post-injection.

Table 8 contains a summary of in vivo local tolerability effects of subcutaneous doses of Microparticle Formulations A, E, G, U, V, and W-2 and comparator placebo microparticles.

TABLE 8
Local Tolerability Effects Summary

| | Group: | | |
|---|---|---|---|
| | Microparticle | Microparticle | |
| Formulations | Formulations | Placebo | |
| Criteria | U, V, and W-2 | A, E, and G | Comparators |
| Average injection | 1.6 to 1.8 cm | 2.0 to 2.2 cm | 1.4 to 1.8 cm |
| site size, Day 6 | | | |
| through Day 9 | | | |
| Average injection | 1.2 to 1.6 cm | 1.5 to 2.0 cm | 0 to 1.4 cm |
| site size, Day 14 | | | |
| through Day 15 | | | |
| Average injection | 1.3 to 1.6 cm | 2.2 to 2.3 cm | 1.2 to 1.7 cm |
| site size, | | | |
| Day 1 through | | | |
| Day 28 | | | |

Histological Observations at Day 14:

| | | | |
|---|---|---|---|
| Fibrous | mild | mild | mild |
| Encapsulation | | | |
| Foreign Body | minimal | mild, also mild mixed | mild |
| Inflammation | | cell inflammation | |
| Purulent or | no/minimal | minimal to moderate | no/minimal |
| Inflammatory | | | |
| Exudate | | | |

### EXAMPLE 7

The following example describes an experiment to determine the effect of water and risedronate sodium concentration in the inner emulsion on microparticle performance.

As described supra, microparticles were formed using a process wherein risedronate sodium is dissolved in 70 to 80° C. water at 100 mg/mL, thus reducing the quantity of water added. A first step of the method created an inner mixture by adding aqueous bisphosphonate to the polymer/solvent combination with homogenization. However, as described supra, during this step particulate formation and precipitation had been observed. In one embodiment of the invention, the concentration of bisphosphonate can be lowered below its solubility limit to reduce or substantially eliminate particulate formation and precipitation. This approach can result in more water within the inner emulsion while maintaining the quantity of bisphosphonate.

Several batches of microparticles were formed to determine the effect of water and risedronate sodium concentration in the inner emulsion on microparticle performance. Table 9 summarizes the results. Microparticle Formulations Y, Z and AA were produced as described in Example 1 (at a 5 gram scale) with appropriate variation of the ratio of the water weight (contained in the aqueous solution of risedronate) to the batch size and variation of the bisphosphonate concentration in the aqueous risedronate sodium. Microparticle Formulations BB, CC, DD and EE were produced as described in Example 6 (at a 20 gram scale) with appropriate variation of the ratio of the water weight to the batch size and variation of the risedronate sodium concentration.

TABLE 9
Microparticles Formed by Varying Inner Emulsion Processing Conditions

| | | Ratio | Aqueous | | |
|---|---|---|---|---|---|
| | | Water/ | Risedronate | Risedronate | |
| | | Batch | Sodium | Sodium | 24 hour In Vitro |
| Microparticle | | Size | Concentration | Load | Release |
| Formulation | Polymer | (wt/wt) | (mg/mL) | (wt %) | (% cumulative) |
| Y | 7525 | 0.5 | 100 | 4.3% | 4 |
| | LOW IV | | | | |
| Z | 7525 | 0.7 | 75 | 5.5% | 8 |
| | LOW IV | | | | |
| AA | 75:25 | 1.0 | 50 | 5.1% | 21 |
| | LOW IV | | | | |
| BB | 8515 6A | 0.5 | 100 | 3.0% | 18 |
| CC | 8515 6A | 0.7 | 75 | 2.9% | 33 |
| DD | 100 4M | 0.5 | 100 | 2.7% | 11 |
| EE | 100 4M | 0.7 | 75 | 3.8% | 39 |

Table 9 demonstrates that increased water presence (lower concentration of risedronate sodium) had no apparent effect on risedronate sodium load efficiency, but resulted in significant increase in initial in vitro release of the risedronate from the microparticles.

### EXAMPLE 8

This example describes a microparticle storage stability study.

Microparticle Formulations C (formed by the W/O/O emulsion process) and Q (formed by the W/O/W emulsion process), made as described supra, were stored right-side-up in borosilicate vials (glass), stoppered with rubber closures and hand crimped. The following storage conditions and times were studied: −20° C.: 1, 2 and 3 months; 4° C.: 1, 2 and 3 months; and 25° C.: 2 weeks and 1 month. The microparticles were tested for appearance, aspect (suspension/aspiration/injection), microparticle particle size, and in vitro release.

In terms of appearance, there was no change following storage. The microparticles remained a white, free-flowing powder. In the aspect test there was also no change. The microparticles continued to suspend, aspirate, and inject well. Table 10 demonstrates that there was no significant change in particle size during the study. Initial in vitro release samples at 25° C. remained unchanged as shown in Table 11. At −20 and 4° C., the release remained low.

TABLE 10
Effects of Storage on Microparticle Particle Size

| | Formulation C | | Formulation Q | |
|---|---|---|---|---|
| | Particle Size (microns) | | Particle Size (microns) | |
| Storage Condition/Time | DV₅₀ | DV₉₀ | DV₅₀ | DV₉₀ |
| Bulk Microparticles | 90 | 156 | 80 | 118 |
| t = 0 | 75 | 119 | 80 | 119 |
| −20° C./1 month | 93 | 160 | 82 | 120 |
| −20° C./3 months | 94 | 164 | 84 | 122 |
| 4° C./1 month | 92 | 159 | 83 | 120 |
| 4° C./3 months | 92 | 158 | 85 | 121 |
| 25° C./2 weeks | 94 | 165 | 84 | 123 |
| 25° C./1 month | 91 | 156 | 84 | 121 |

Results reported are an average of n=2 samples per condition except Bulk Microparticles and t=0 (n=1).

TABLE 11
Effect of Storage on Microparticle In Vitro Release

| | Formulation C | | | Formulation Q | | |
|---|---|---|---|---|---|---|
| | % Released | | | % Released | | |
| Storage | 1 | 4 | 24 | 1 | 4 | 24 |
| Condition/Time | hour | hours | hours | hour | hours | hours |
| Bulk | 1.9 | 3.1 | 4.6 | 0.6 | 1.1 | 1.9 |
| Microparticles | | | | | | |
| −20° C./1 month | 1.9 | 3.1 | 5.8 | 1.3 | 2.3 | 3.3 |
| −20° C./3 months | 2.4 | 4.0 | 7.4 | 2.2 | 4.1 | 6.2 |
| 4° C./1 month | 1.8 | 3.8 | 7.1 | 1.0 | 1.8 | 2.6 |
| 4° C./3 months | 2.8 | 4.4 | 6.4 | 2.2 | 4.0 | 6.2 |
| 25° C./2 weeks | 1.4 | 2.2 | 3.6 | 0.9 | 1.3 | 1.5 |
| 25° C./1 month | 1.9 | 3.0 | 4.5 | 1.0 | 1.8 | 2.7 |

Results reported are an average of n=2 samples per condition.

### EXAMPLE 9

This example describes the formation of 5 gram batches of microparticles containing risedronate sodium and a biocompatible polymer using a W/O/W emulsion process wherein the inner emulsion includes a surfactant.

2.5 mL of RODI water was mixed with 25 milligrams of a surfactant (i.e., polyvinyl alcohol (PVA), Pluronic F68 or TWEEN® 20) to form a 0.1% (w/v) surfactant solution. The surfactant solution was then added to a 20 mL glass scintillation vial containing 290 mg of risedronate sodium BDS. The vial was then placed in an about 80° C. water bath. The BDS was dissolved by swirling while the temperature was maintained at 80° C., thereby forming an aqueous solution of risedronate at a concentration of about 100 mg risedronate sodium/mL solution. The aqueous solution of risedronate was then allowed to cool to ambient temperature. 4.75 g of a MEDISORB® polymer were mixed with about 21.6 to about 34.8 g of ethyl acetate, thereby forming a polymer/ethyl acetate combination with about 12 to about 18 weight percent polymer. The polymer/ethyl acetate combination was poured into a stainless steel funnel with a valve on the bottom. A sonication microtip probe (Model No. CV17; Sonics and Materials, Inc., Danbury, Conn.) was placed about 1 cm below the surface of the polymer/ethyl acetate combination. 2.5 mL of the ambient temperature aqueous solution of risedronate was drawn into a sterile 3 mL syringe with a 1.5 inch (about 3.8 cm), 18 gauge needle. The sonication was turned on at 40% amplitude. The aqueous solution of risedronate was then injected near the microtip probe over an injection time of 14 sec+/−5 sec. Sonication of the resulting mixture was continued for about 1 minute after the end of injection. Thus, a primary, or inner, emulsion (W/O) was formed. The inner emulsion was observed to be stable with uniformly suspended particles that were less than about 8 microns in size, based on visual observation under a light microscope.

A 0.25 in (about 0.64 cm) outside diameter, 34 element, static mixer constructed of 316 stainless steel (Model No. 04669-60; Cole-Parmer Instrument Co., Vernon Hills, Ill.) was primed for about 5 seconds by pumping a polyvinyl alcohol (PVA) solution, containing 1 weight percent PVA with 6.5 weight percent ethyl acetate, through a T-junction and into the mixer at about 700 mL/min. Following priming, the flow rate of the PVA solution was maintained at about 700 mL/min. The primary emulsion was then pumped through the other branch of the T-junction at a flow rate of 70 mL/min to combine with the PVA solution stream. The combined streams were directed into the static mixer. The primary emulsion was shunted into the PVA solution for about 30+/−5 seconds. Five seconds after the addition of the primary emulsion, the PVA solution stream was turned off.

The static mixer outlet was joined to a dip-tube which emptied into a quench tank. As the mixture of the primary emulsion and PVA solution left the static mixer, the combined stream flowed into the quench tank. The quench tank was initially charged with 3.5 liters of RODI water at room temperature. The quench tank was equipped with an impeller stirring at about 300 to about 400 rpm. Following addition of the combined primary emulsion and PVA solution streams, the quench tank was stirred for 30 minutes.

After 30 minutes of residence in the quench tank, the contents of the tank were directed into a 25 micron stainless steel sieve (diameter of 21 cm) to collect the microparticles. The microparticles were washed in the sieve with a continuous flow of RODI water for about 3 to 5 minutes. The microparticles were transferred to a sterile glass dish with a diameter of about 3 to 5 cm and the dish was covered with a Kimwipe. The glass dish was placed in a freezer at −80° C. for at least about 30 minutes. The glass dish was then placed in a pre-chilled lyophilizer (Model No. TD-2C-MP; FTS Systems, Inc., Stone Ridge, N.Y.) with a shelf temperature of about −40° C. The following lyophilization program was then performed: started at −40° C., 150 millitorr (mT); ramped 2.5° C./min to −10° C.; held 5 hours at 300 mT; ramped 2.5° C./min to 30° C.; and held for 2 days at 300 mT. The microparticles were then poured into a sterilized 150 micron stainless steel sieve (6 cm diameter) and the microparticles were sieved by banging and breaking microparticle masses with a spatula. The fraction of the microparticles that passed through the sieve were collected as the microparticle product.

A control microparticle formulation was also prepared generally as described above, but that contained no surfactant.

The addition of surfactant to the aqueous phase of the inner emulsion resulted in significantly higher percent incorporation (wt/theory wt) of risedronate sodium of about 95 to about 100 weight percent compared to only 75 weight percent encapsulation for the control microparticles that were formed without surfactant. Also, microparticles formed using a surfactant in the inner emulsion did not have significantly affected initial in vitro release. The results of these experiments are shown in Table 12.

TABLE 12
Microparticles Formed by Including a Surfactant in an Inner Emulsion

| | | | | 24-hour |
|---|---|---|---|---|
| | | | Incorpora- | In |
| | Surfactant | Risedronate | tion | Vitro |
| | in | Sodium | Efficiency | Release |
| Microparticle | Inner | Load | (% wt/theory | (Cumulative |
| Formulation | Emulsion | (wt %) | wt) | %) |
| FF | none | 3.76 | 75 | 11 |
| GG | PVA | 4.75 | 95 | 12 |
| HH | Pluronic | 4.86 | 97 | 5 |
| | F68 | | | |
| II | TWEEN ® | 4.99 | 100 | 5 |
| | 20 | | | |

### EXAMPLE 10

This example describes the production of microparticles containing risedronate sodium and a biocompatible polymer using a W/O/W emulsion process.

2.5 milliliters (mL) of reverse osmosis deionized (RODI) water was added to a 20 mL glass scintillation vial containing 290 milligrams (mg) of risedronate sodium bulk drug substance (BDS). The vial was then placed in an 80° C. water bath. The BDS was dissolved by swirling while the temperature was maintained at 80° C., thereby forming an aqueous solution of risedronate at a concentration of about 100 milligrams risedronate sodium/milliliter solution (mg/mL).

4.75 grams (g) of a MEDISORB® polymer were mixed with 21.6 to 34.8 g of ethyl acetate, thereby forming a polymer/ethyl acetate combination with about 12 to about 18 weight percent polymer. Table 13 shows specific polymers and polymer concentrations used for several microparticle formulations.

TABLE 13
Microparticle Formulations

| | | Polymer Concentration in | |
|---|---|---|---|
| | | Polymer/Solvent | Emulsion Flow |
| Formulation | Polymer | Combination | Rate (mL/min) |
| H-1 | 6535 LOW IV | 15 wt % | 770 |
| H-2 | | | 1100 |
| H-3* | | | 1650 |
| Q-1 | 7525 HIGH IV | 12 wt % | 770 |
| Q-2 | | | 1100 |
| Q-3* | | | 1650 |
| S-1 | 100 4M | 18 wt % | 770 |
| S-2 | | | 1100 |
| S-3 | | | 1100 |
| S-4* | | | 1650 |

*Two, 34-element static mixer assemblies connected in series were used.

The polymer/ethyl acetate combination was poured into a stainless steel funnel with a valve on the bottom. A sonication microtip probe (Model No. CV17; Sonics and Materials, Inc., Danbury, Conn.) was placed about 1 centimeter (cm) below the surface of the polymer/ethyl acetate combination. 2.5 mL of the 80° C. aqueous solution of risedronate was drawn into a sterile 3 mL syringe with a 1.5 in (about 3.8 cm), 18 gauge needle. The sonication was turned on at 40% amplitude. The 80° C. aqueous solution of risedronate was then injected near the microtip probe over an injection time of 14 sec+/−5 sec. Sonication of the resulting mixture was continued for about 1 minute after the end of injection. Thus, a primary, or inner, emulsion (W/O) was formed.

A 0.25 in (about 0.64 cm) outside diameter, 34 element, static mixer constructed of 316 stainless steel (Model No. 04669-60; Cole-Parmer Instrument Co., Vernon Hills, IL) was primed for about 5 seconds by pumping a polyvinyl alcohol (PVA) solution, containing 1 weight percent PVA with 6.5 weight percent ethyl acetate, through a T-junction and into the mixer. Following priming, the flow rate of the PVA solution was maintained. The primary emulsion was then pumped through the other branch of the T-junction to combine with the PVA solution stream. The combined streams were directed into the static mixer. The primary emulsion was shunted into the PVA solution for about 30+/−5 seconds. Five seconds after the addition of the primary emulsion, the PVA solution stream was turned off. The total flow rate through the static mixer for each of several microparticle formulations is shown in Table 13.

The static mixer outlet was joined to a dip-tube which emptied into a quench tank. As the mixture of the primary emulsion and PVA solution left the static mixer, the combined stream flowed into the quench tank. The quench tank was initially charged with 3.5 liters of RODI water at room temperature. The quench tank was equipped with an impeller stirring at about 300 to about 400 rpm. Following addition of the combined primary emulsion and PVA solution streams, the quench tank was stirred for 30 minutes.

After 30 minutes of residence in the quench tank, a valve on the bottom of the quench tank was opened and the contents of the tank were directed into a 25 micron stainless steel sieve (diameter of 21 cm) to collect the microparticles. The microparticles were washed in the sieve with a continuous flow of RODI water for about 3 to 5 minutes. The microparticles were transferred to a sterile glass dish with a diameter of about 3 to 5 cm and the dish was covered with a Kimwipe. The glass dish was placed in a freezer at −80° C. for at least about 30 minutes. The glass dish was then placed in a pre-chilled lyophilizer (Model No. TD-2C-MP; FTS Systems, Inc., Stone Ridge, N.Y.) with a shelf temperature of about −40° C. The following lyophilization program was then performed: started at −40° C., 150 millitorr (mT); ramped 2.5° C./min to −10° C.; held 5 hours at 300 mT; ramped 2.5° C./min to 30° C.; and held for 2 days at 300 mT. The microparticles were then poured into a sterilized 150 micron stainless steel sieve (6 cm diameter) and the microparticles were sieved by banging and breaking microparticle masses with a spatula. The fraction of the microparticles that passed through the sieve were collected as the microparticle product.

Table 14 shows several performance characteristics of the microparticles produced using this method. Without being held to any particular theory, it is believed that by increasing flow through the static mixer, greater shear is generated during the combination of the primary emulsion stream and the PVA solution stream, resulting in smaller microparticle size.

TABLE 14
Microparticle Characteristics

| | Particle | | | |
|---|---|---|---|---|
| | Size | | | 24 hour In Vitro |
| | (microns) | | Risedronate Sodium | Release |
| Formulation | DV₅₀ | DV₉₀ | Content (wt %) | (Cumulative %) |
| H-1 | 81 | 115 | 3.3 | 3 |
| H-2 | 56 | 83 | 3.8 | 14 |
| H-3 | 41 | 61 | 5.2 | 44 |
| Q-1 | 80 | 115 | 3.6 | 3 |
| Q-2 | 58 | 86 | 4.2 | 11 |
| Q-3 | 39 | 54 | 4.3 | 21 |
| S-1 | 79 | 113 | 4.9 | 9 |
| S-2 | 56 | 81 | 4.4 | 37 |
| S-3 | 58 | 81 | 4.1 | 36 |
| S-4 | 39 | 52 | 6.2 | 41 |

By increasing the flow rate through the static mixer, smaller microparticles were produced. The initial in vitro releases of the 7525 HIGH IV formulations were considered acceptable for further experimentation. The 6535 LOW IV and the 100 4M formulations were further modified to improve initial in vitro release. Two process modifications were identified that improved initial in vitro release in the 6535 LOW IV and the 100 4M formulations. These modifications were elevating the polymer solution temperature from ambient temperature to 65° C. and/or adding 0.05% dry weight Pluronic F68 to the aqueous solution of risedronate.

### EXAMPLE 11

This example describes the production of microparticles containing risedronate sodium and a biocompatible polymer using a W/O/W emulsion process.

2.5 milliliters (mL) of reverse osmosis deionized (RODI) water was added to a 20 mL glass scintillation vial containing 290 milligrams (mg) of risedronate sodium bulk drug substance (BDS). The vial was then placed in an 80° C. water bath. The BDS was dissolved by swirling while the temperature was maintained at 80° C., thereby forming an aqueous solution of risedronate at a concentration of about 100 milligrams risedronate sodium/milliliter solution (mg/mL).

4.75 grams (g) of a MEDISORB® polymer were mixed with 21.6 to 34.8 g of ethyl acetate, thereby forming a polymer/ethyl acetate combination with about 12 to about 18 weight percent polymer. Table 15 shows specific polymers and polymer concentrations used for three microparticle formulations.

TABLE 15
Microparticle Formulations

| | | Static Mixer | Polymer Concentration |
|---|---|---|---|
| | | Flow Rate | in Polymer/Solvent |
| Formulation | Polymer | (mL/min) | Combination |
| H-4 | 6535 LOW IV | 1100 | 15 wt % |
| Q-4 | 7525 HIGH IV | 1100 | 12 wt % |
| S-5 | 100 4M | 1100 | 18 wt % |

The polymer/ethyl acetate combination was heated to about 60° C. to 65° C. The heated polymer/ethyl acetate combination was poured into a stainless steel funnel with a valve on the bottom. A sonication microtip probe (Model No. CV17; Sonics and Materials, Inc., Danbury, Conn.) was placed about 1 centimeter (cm) below the surface of the polymer/ethyl acetate combination. 2.5 mL of the 80° C. aqueous solution of risedronate was drawn into a sterile 3 mL syringe with a 1.5 in (about 3.8 cm), 18 gauge needle. The sonication was turned on at 40% amplitude. The 80° C. aqueous solution of risedronate was then injected near the microtip probe over an injection time of 14 sec+/−5 sec. Sonication of the resulting mixture was continued for about 1 minute after the end of injection. Thus, a primary, or inner, emulsion (W/O) was formed.

A 0.25 in (about 0.64 cm) outside diameter, 34 element, static mixer constructed of 316 stainless steel (Model No. 04669-60; Cole-Parmer Instrument Co., Vernon Hills, Ill.) was primed for about 5 seconds by pumping a polyvinyl alcohol (PVA) solution, heated to about 60° C. to about 65° C. and containing 1 weight percent PVA with 6.5 weight percent ethyl acetate, through a T-junction and into the mixer at a flow rate of 1000 mL/min. Following priming, the flow rate of the PVA solution was maintained at 1000 mL/min. The primary emulsion was then pumped through the other branch of the T-junction at a flow rate of 100 mL/min to combine with the PVA solution stream. The combined streams were directed into the static mixer. The primary emulsion was shunted into the PVA solution for about 30+/−5 seconds. Five seconds after the addition of the primary emulsion, the PVA solution stream was turned off. The total flow rate through the static mixer was 1100 mL/min.

The static mixer outlet was joined to a dip-tube which emptied into a quench tank. As the mixture of the primary emulsion and PVA solution left the static mixer, the combined stream flowed into the quench tank. The quench tank was initially charged with 3.5 liters of RODI water at room temperature. The quench tank was equipped with an impeller stirring at about 300 to about 400 rpm. Following addition of the combined primary emulsion and PVA solution streams, the quench tank was stirred for 30 minutes.

After 30 minutes of residence in the quench tank, a valve on the bottom of the quench tank was opened and the contents of the tank were directed into a 25 micron stainless steel sieve (diameter of 21 cm) to collect the microparticles. The microparticles were washed in the sieve with a continuous flow of RODI water for about 3 to 5 minutes. The microparticles were transferred to a sterile glass dish with a diameter of about 3 to 5 cm and the dish was covered with a Kimwipe. The glass dish was placed in a freezer at −80° C. for at least about 30 minutes. The glass dish was then placed in a pre-chilled lyophilizer (Model No. TD-2C-MP; FTS Systems, Inc., Stone Ridge, N.Y.) with a shelf temperature of about −40° C. The following lyophilization program was then performed: started at −40° C., 150 millitorr (mT); ramped 2.5° C./min to −10° C.; held 5 hours at 300 mT; ramped 2.5° C./min to 30° C.; and held for 2 days at 300 mT. The microparticles were then poured into a sterilized 150 micron stainless steel sieve (6 cm diameter) and the microparticles were sieved by banging and breaking microparticle masses with a spatula. The fraction of the microparticles that passed through the sieve were collected as the microparticle product.

Table 16 shows a comparison of microparticles produced as in Example 10 (H-3, Q-3, and S-4) and those microparticles produced using the method of the present Example (H-4, Q-4, and S-5). Although both approaches yield microparticles with similar size distributions, microparticle formulations H-4, Q-4, and S-5 had comparatively lower initial in vitro release rates.

TABLE 16
Microparticles Comparisons

| | | | Risedronate | 24 hour In Vitro |
|---|---|---|---|---|
| | Particle Size (microns) | | Sodium | Release |
| Formulation | DV₅₀ | DV₉₀ | Content (wt %) | (Cumulative %) |
| H-3 | 41 | 61 | 5.2 | 44 |
| H-4 | 39 | 48 | 4.2 | 8 |
| Q-3 | 39 | 54 | 4.3 | 21 |
| Q-4 | 37 | 47 | 3.1 | 3 |
| S-4 | 39 | 52 | 6.2 | 41 |
| S-5 | 40 | 53 | 5.7 | 14 |

### EXAMPLE 12

This example describes the production of microparticles at a 60 gram scale containing risedronate sodium and a biocompatible polymer using a W/O/W emulsion process.

30 mL of RODI water was mixed with 30 mg of the surfactant Pluronic F68 to form a 0.1% (w/v) surfactant solution. The surfactant solution was then added to a 50 mL polypropylene tube containing 3.48 g of risedronate sodium BDS. The vial was then placed in an about 80° C. water bath. The BDS was dissolved by swirling while the temperature was maintained at 80° C., thereby forming an aqueous solution of risedronate at a concentration of about 100 mg risedronate sodium/mL solution. The aqueous solution of risedronate was then allowed to cool to ambient temperature. The aqueous solution of risedronate had a concentration of about 100 mg risedronate sodium/mL solution.

57 grams (g) of 6535 LOW IV polymer were mixed with 323 g of ethyl acetate, thereby forming a polymer/ethyl acetate combination with about 15 weight percent polymer. The polymer/ethyl acetate combination was heated to about 60° C. to 65° C. in a jacketed stainless steel funnel with a valve on the bottom. AN IKA T25 homogenizer (Wilmington, N.C.) was placed about 2 centimeters (cm) above the bottom of the vessel containing the polymer/ethyl acetate combination. 30 mL of the 80° C. aqueous solution of risedronate was poured into a jacketed stainless steel 100 mL funnel with a valve on the bottom and a stainless steel tube extending to 1 cm above the polymer vessel bottom. The homogenizer was turned to a high speed (2400 rpm). The valve below the stainless steel funnel containing 80° C. aqueous solution of risedronate was then opened and drug solution was allowed to flow through the tube into the polymer solution over a time of 1 to 2 min. Homogenization of the resulting mixture was continued for about 2-4 minutes after the end of injection. Thus, a primary, or inner, emulsion (W/O) was formed.

A 0.25 in (about 0.64 cm) outside diameter, 34 element, static mixer constructed of 316 stainless steel (Model No. 04669-60; Cole-Parmer Instrument Co., Vernon Hills, Ill.) was primed for about 5 seconds by pumping a polyvinyl alcohol (PVA) solution containing 1 weight percent PVA with 6.5 weight percent ethyl acetate, through a T-junction and into the mixer at a flow rate of 1200 mL/min. Following priming, the flow rate of the PVA solution was maintained at 1200 mL/min. The primary emulsion was then pumped through the other branch of the T-junction at a flow rate of 120 mL/min to combine with the PVA solution stream. The combined streams were directed into the static mixer. The primary emulsion was shunted into the PVA solution for about 1320 seconds. Fifteen seconds after the addition of the primary emulsion, the PVA solution stream was turned off. The total flow rate through the static mixer was 1320 mL/min.

The static mixer outlet was joined to a dip-tube which emptied into a quench tank. As the mixture of the primary emulsion and PVA solution left the static mixer, the combined stream flowed into the quench tank. The quench tank was initially charged with 45 liters of RODI water at room temperature. The quench tank was equipped with an impeller stirring at about 143 rpm. Following addition of the combined primary emulsion and PVA solution streams, the quench tank was stirred for 1 hour.

After approximately 60 minutes of residence in the quench tank, a valve on the bottom of the quench tank was opened and the contents of the tank were directed into a filter dryer (ITT Sherotec, Simi Valley, Calif.) to collect the microparticles. The microparticles were washed in the filter dryer with 3×15 L of RODI water and dewatered and denatured in place. The microparticles were then split into various fractions and vacuum dried under various temperature conditions for at least three days. One portion of the microparticles (Formulation JJ) was transferred to a sterile glass dish and the dish was covered with a Kimwipe and placed in a lyophilizer (FTS) at 0° C. The remainder of the material was dried in place on a filter drier for 20 hours at 10° C. and then for 4 days at 25° C. A mixer in the filter dryer chamber was used throughout the drying process (Lightnin, Rochester, N.Y.). A portion was then removed (Formulation KK). The remaining material in the filter dryer was dried for an additional day at 30° C.

Following drying, the microparticles were poured into a sterilized 150 micron stainless steel sieve and the microparticles were sieved by banging and breaking microparticle masses with a spatula. The fraction of the microparticles that passed through the sieve were collected as the microparticle product. Table 17 illustrates the effects of vacuum drying temperature on microparticle characteristics.

Table 17 indicates that vacuum drying at 25 and 30° C. produced particles with acceptable particle size and initial in vitro release. Drying at 0° C. appears to have produced some agglomeration as indicated by the increase in DV₉₀ particle size. For comparison, a 5 gram scale batch of the microparticles (Formulation MM) was also produced from the same compositions as these 60 gram scale microparticles. The method used to produce these particles is described in Example 9. Formulations JJ, KK, and LL each had a lower 24 hour in vitro release, suggesting that vacuum drying is preferred over the freeze drying to which the microparticles of Formulation MM were subjected.

TABLE 17
Drying Effects on Microparticle Characteristics

| | | Particle Size | | 24 hour In Vitro |
|---|---|---|---|---|
| | Drying Conditions | (microns) | | Release |
| Formulation | and Temperature | DV₅₀ | DV₉₀ | (Cumulative %) |
| JJ | Dewatered on filter | 59 | 117 | 2 |
| | dryer; transferred to | | | |
| | lyophilizer; 0° C., | | | |
| | 3 days | | | |
| KK | Dewatered on filter | 57 | 98 | 2 |
| | dryer; 10° C./25° C., | | | |
| | 4 days | | | |
| LL | Dewatered on filter | 58 | 96 | 3 |
| | dryer; | | | |
| | 10° C./25° C./30° C., | | | |
| | 5 days | | | |
| MM | Filtered and frozen at | 56 | 82 | 6 |
| (5-g scale) | −80° C.; | | | |
| | −40° C./−10° C./30° C. | | | |
| | 3 days | | | |

### EXAMPLE 13

This example describes the production of microparticles at a 60 gram scale containing risedronate sodium and a biocompatible polymer using a W/O/W emulsion process.

30 milliliters (mL) of reverse osmosis deionized (RODI) water was added to a 50 mL polypropylene tube containing 3.48 grams (g) of risedronate sodium bulk drug substance (BDS). The tube was then placed in an 80° C. water bath. The BDS was dissolved by swirling while the temperature was maintained at 80° C., thereby forming an aqueous solution of risedronate at a concentration of about 100 milligrams risedronate sodium/milliliter solution (mg/mL).

57 grams (g) of a MEDISORB® polymer were mixed with 418 to 260 g of ethyl acetate, thereby forming a polymer/ethyl acetate combination with about 12 to about 18 weight percent polymer. Table 18 shows specific polymers and polymer concentrations used for three microparticle formulations.

TABLE 18
Microparticle Formulations

| | | Polymer Concentration in |
|---|---|---|
| Formulation | Polymer | Polymer/Solvent Combination |
| H-5 | 6535 LOW IV | 15 wt % |
| Q-5 | 7525 HIGH IV | 12 wt % |
| S-6 | 100 4M | 18 wt % |

The polymer/ethyl acetate combination was heated to about 60° C. to 65° C. The heated polymer/ethyl acetate combination was poured into a stainless steel funnel with a valve on the bottom. An IKA T25 homogenizer (Wilmington, N.C.) was placed about 2 centimeters (cm) above the bottom of the vessel with the polymer/ethyl acetate combination. 30 mL of the 80° C. aqueous solution of risedronate was poured into a jacketed stainless steel 100 mL funnel with a valve on the bottom and a stainless steel tube extending to 1 cm above the polymer vessel bottom. The homogenizer was turned to a high speed (2400 rpm). The valve below the stainless steel funnel containing 80° C. aqueous solution of risedronate was then opened and drug solution was allowed to flow through the tube into the polymer solution over a time of 1 to 2 min. Homogenization of the resulting mixture was continued for about 2-4 minutes after the end of injection. Thus, a primary, or inner, emulsion (W/O) was formed.

A 0.25 in (about 0.64 cm) outside diameter, 34 element, static mixer constructed of 316 stainless steel (Model No. 04669-60; Cole-Parmer Instrument Co., Vernon Hills, Ill.) was primed for about 5 seconds by pumping a polyvinyl alcohol (PVA) solution, heated to about 60° C. to about 65° C. and containing 1 weight percent PVA with 6.5 weight percent ethyl acetate, through a T-junction and into the mixer at a flow rate of 1200 mL/min. Following priming, the flow rate of the PVA solution was maintained at 1200 mL/min. The primary emulsion was then pumped through the other branch of the T-junction at a flow rate of 120 mL/min to combine with the PVA solution stream. The combined streams were directed into the static mixer. The primary emulsion was shunted into the PVA solution for about 2 to 5 min. Five seconds after the addition of the primary emulsion, the PVA solution stream was turned off. The total flow rate through the static mixer was 1320 mL/min.

The static mixer outlet was joined to a dip-tube which emptied into a quench tank. As the mixture of the primary emulsion and PVA solution left the static mixer, the combined stream flowed into the quench tank. The quench tank was initially charged with 45 liters of RODI water at room temperature. The quench tank was equipped with an impeller stirring at about 300 to about 400 rpm. Following addition of the combined primary emulsion and PVA solution streams, the quench tank was stirred for 30 minutes.

After 60 minutes of residence in the quench tank, a valve on the bottom of the quench tank was opened and the contents of the tank were directed into a custom built filter dryer (ITT Sherotec, Simi Valley, Calif.) to collect the microparticles. The microparticles were washed in the filter dryer with 3×15 L of RODI water and dewatered in place. The microparticles were then dried at 8 Torr either in a lyophilizer or in place in the filter dryer for at least three days. One batch (Formulation H-5) was transferred to a sterile glass dish, the dish was covered with a Kimwipe and the material was at dried 10° C. for 16 hours, and then at 25° C. for three days in a lyophilizer (FTS).

The other two batches (Formulations S-6 and Q-5) were dried in place on a custom built filter drier at 10° C. overnight and then for 3 days at 25° C. A mixer in the filter dryer chamber was used throughout the drying process (Lightnin, Rochester, N.Y.). The microparticles were then poured into a sterilized 8 inch, 150 micron stainless steel sieve and sieved for 10 minutes using a Gilson Sieve Shaker autosieve, Model GA-8 (Lewis Center, Ohio). The fraction of the microparticles that passed through the sieve were collected as the microparticle product.

Table 19 shows characteristics of microparticles produced using this method.

TABLE 19
Microparticle Characteristics

| | | | Risedronate | 24 hour In Vitro |
|---|---|---|---|---|
| | Particle Size (microns) | | Sodium | Release |
| Formulation | DV₅₀ | DV₉₀ | Content (wt %) | (Cumulative %) |
| H-5 | 33 | 52 | 2.8 | 9 |
| Q-5 | 32 | 46 | 3.0 | 11 |
| S-6 | 34 | 51 | 3.4 | 15 |

### EXAMPLE 14

This example describes the production of microparticles containing risedronate sodium and a biocompatible polymer using a W/O/W emulsion process.

Table 20 shows several microparticle formulations that were produced. Microparticle Formulations MM, KK, and Q-2 were produced as described supra. Formulation NN was produced at a 5 gram scale using a method similar to that described in Example 11 but instead using an ambient temperature PVA solution. Formulation OO was produced using a method similar to that described in Example 12 with vacuum drying at 25° C. Formulation PP was produced using a method similar to that described in Example 12 except that the polymer/solvent combination was not heated to 60° C. to 65° C., but was instead maintained at ambient temperature, and with vacuum drying at 25° C. Formulation QQ was produced at a 5 gram scale using a method similar to that described in Example 9 except that the polymer/solvent combination was heated to about 65° C. prior to formation of the primary emulsion. Microparticle Formulation RR was formed using a method similar to that described in Example 12 with vacuum drying at 25° C.

TABLE 20
Microparticle Formulations

| | | Polymer | Temperature | Surfactant |
|---|---|---|---|---|
| | | Concentration in | of Polymer/ | Present in |
| Formu- | | Polymer/Solvent | Solvent | Drug |
| lation | Polymer | Combination | Combination | Solution |
| NN | 6535 LOW IV | 15 wt % | 65° C. | None |
| OO | 6535 LOW IV | 15 wt % | 65° C. | None |
| MM* | 6535 LOW IV | 15 wt % | Ambient | Pluronic F68 |
| KK* | 6535 LOW IV | 15 wt % | Ambient | Pluronic F68 |
| Q-2* | 7525 HIGH IV | 12 wt % | Ambient | None |
| PP | 7525 HIGH IV | 12 wt % | Ambient | None |
| QQ | 100 4M | 18 wt % | 65° C. | Pluronic F68 |
| RR | 100 4M | 18 wt % | 65° C. | Pluronic F68 |

*Produced as described supra

Table 21 lists characteristics of the microparticles produced as described in Table 20. Formulations OO, KK and Q-2 all exhibited good initial in vitro release, particle size, and drug load. However, the drug load of Formulation PP was lower than expected. Further experiments related to the drug load of Formulation PP are described in Example 15.

TABLE 21
Microparticle Characteristics

| | | Particle | | | |
|---|---|---|---|---|---|
| | | Size | | Risedronate | 24 hour In Vitro |
| | Scale | (microns) | | Sodium | Release |
| Formulation | (g) | DV₅₀ | DV₉₀ | Content (wt %) | (Cumulative %) |
| NN | 5 | 56 | 81 | 4.7 | 7 |
| OO | 60 | 57 | 89 | 3.4 | 2 |
| MM | 5 | 56 | 82 | 4.7 | 6 |
| KK | 60 | 57 | 98 | 4.1 | 2 |
| Q-2 | 5 | 58 | 86 | 4.2 | 11 |
| PP | 60 | 49 | 73 | 1.0 | 2 |
| QQ | 5 | 52 | 75 | 5.2 | 10 |
| RR | 60 | 50 | 78 | 4.3 | 11 |

### EXAMPLE 15

This example describes the production of microparticles containing risedronate sodium and 7525 HIGH IV polymer using a W/O/W emulsion process at a 60 gram batch scale.

Table 22 shows various microparticle formulation conditions that were used to produce microparticles containing 7525 HIGH IV polymer. In general, microparticles were produced using a method similar to that of Example 13 but with the polymer/solvent combination kept at ambient temperature and with the aqueous solution of risedronate having a drug concentration of 100 mg/mL (exceptions are noted in Table 21).

TABLE 22
Microparticle Formulations

| | | Drug | |
|---|---|---|---|
| | Polymer Concentration | Addition | |
| | in Polymer/Solvent | Time | Homogenization Time |
| Formulation | Combination | (minutes) | (minutes) |
| PP (control) | 12 wt % | 2 | 4 |
| ¹PP-1 | 12 wt % | 1 | 1 |
| PP-2 | 12 wt % | 1 | 1 |
| ²PP-3 | 12 wt % | 2 | 4 |
| PP-4 | 15 wt % | 2 | 4 |
| PP-5 | 15 wt % | 1 | 1 |
| ³PP-6 | 12 wt % | 1 | 1 |
| PP-7 | 10 wt % | 1 | 1 |
| PP-8 | 18 wt % | 1 | 1 |

¹A large scale sonication homogenization method was used for this batch, Sonics
and Materials Model VC-750 generator with Model A07109PRB probe (Newtown, CT).
²Temperature of polymer/solvent combination was 60° C.
³Drug concentration was 75 mg/mL

Table 23 lists characteristics of the microparticles thus formed. Microparticles having better characteristics were those produced using methods that involved reducing the drug addition rate and homogenization time; increasing the polymer concentration (e.g., from 12 wt % to 15 wt % or 18 wt %); and reducing the drug concentration (e.g., from 100 mg/mL to 75 mg/mL).

TABLE 23
Microparticle Characteristics

| | | | 24 hour In Vitro | Risedronate |
|---|---|---|---|---|
| | Particle Size (microns) | | Release | Sodium |
| Formulation | DV₅₀ | DV₉₀ | (Cumulative %) | Content (wt %) |
| PP (control) | 49 | 73 | 2 | 1.0 |
| PP-1 | 67 | 116 | 3 | 1.4 |
| PP-2 | 49 | 73 | 3 | 2.2 |
| PP-3 | 52 | 79 | 2 | 1.4 |
| PP-4 | 62 | 99 | 2 | 3.1 |
| PP-5 | 59 | 94 | 3 | 3.7 |
| PP-6 | 55 | 86 | 3 | 4.3 |
| PP-7 | 47 | 73 | 2 | 1.1 |
| PP-8 | 49 | 105 | 2 | 4.3 |

### EXAMPLE 16

This example describes in vivo release of risedronate sodium in rats from several microparticles produced as described supra.

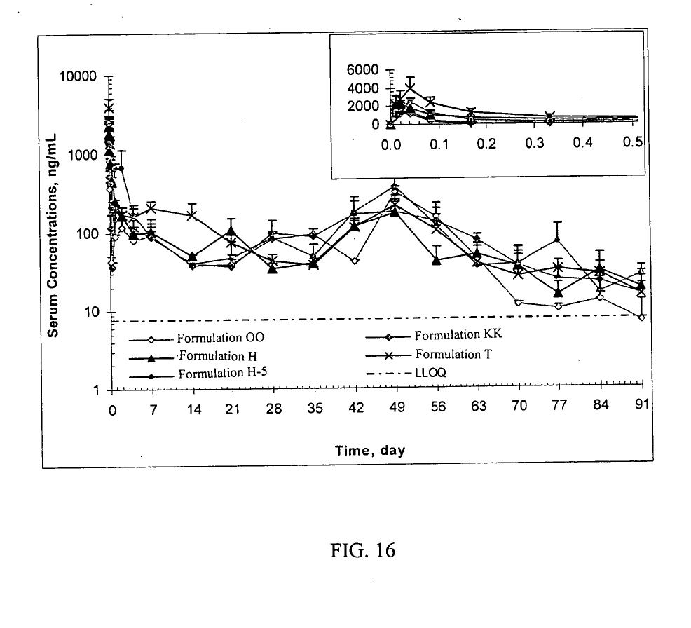

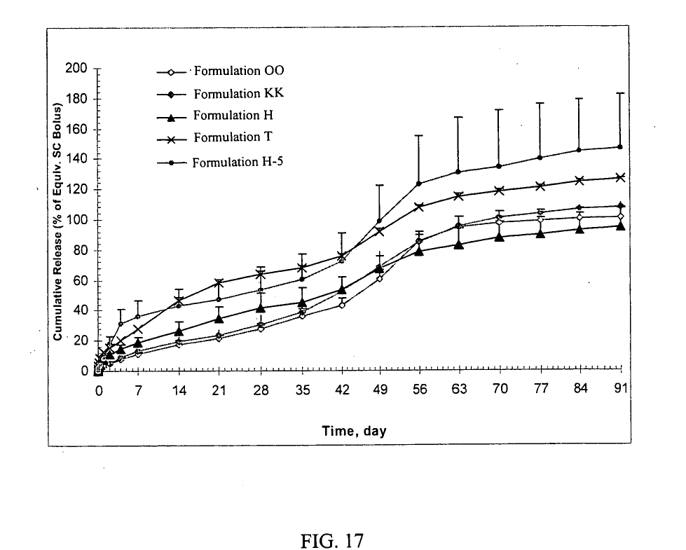

FIGS. 16, 18, and 20 show PK plots tracking sustained drug levels in rat serum as a function of time for each formulation. FIGS. 17, 19, and 21 show cumulative AUC versus time.

FIG. 16 shows a comparison of blood serum concentration in rat for Microparticle Formulations OO, H, H-5, KK, and T, each containing 6535 LOW IV polymer.

FIG. 17 shows a cumulative release profile in rat for Microparticle Formulations OO, H, H-5, KK, and T, each containing 6535 LOW IV polymer.

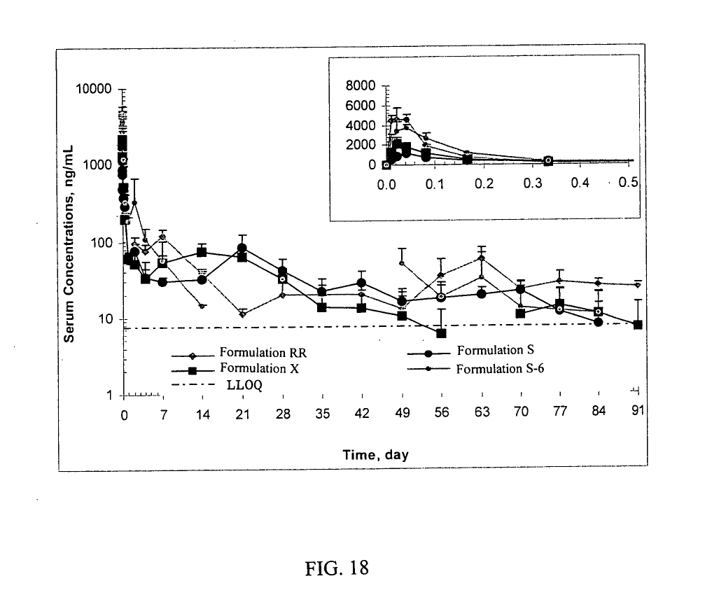

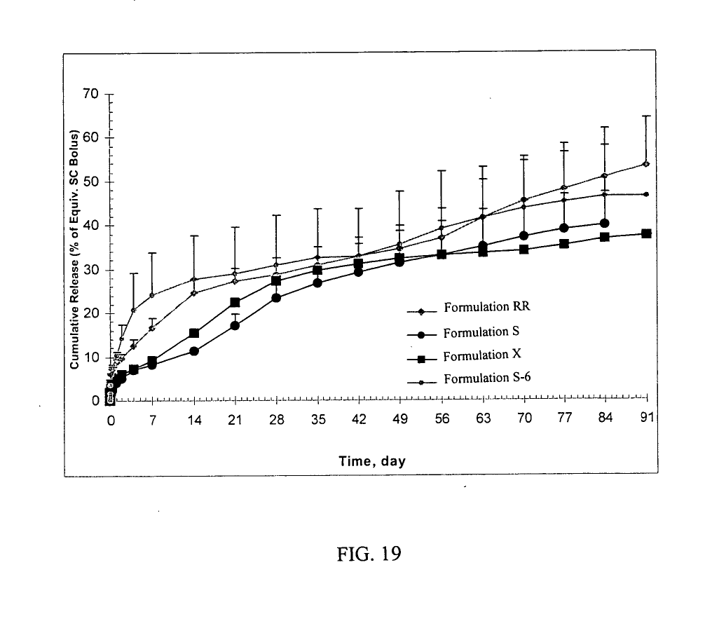

FIG. 18 shows a comparison of blood serum concentration in rat for Microparticle Formulations RR, X, S, and S-6, each containing 100 4M polymer. FIG. 19 shows a cumulative release profile in rat for Microparticle Formulations RR, X, S, and S-6, each containing 100 4M polymer.

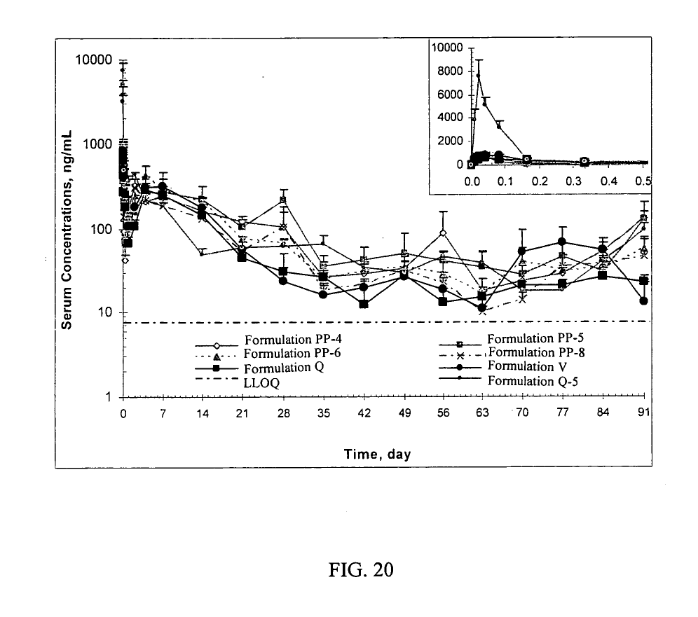

FIG. 20 shows a comparison of blood serum concentration in rat for Microparticle Formulations PP-4, PP-6, Q, PP-5, PP-8, V, and Q-5, each containing 7525 HIGH IV polymer.

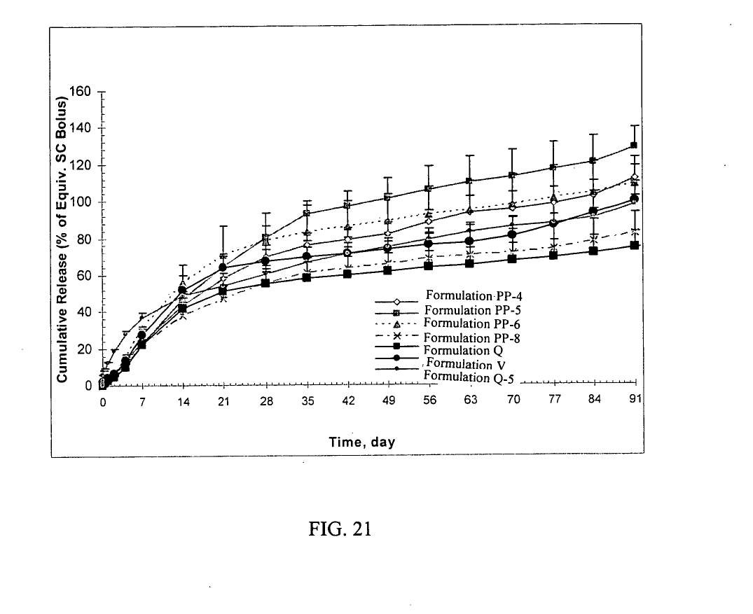

FIG. 21 shows a cumulative release profile in rat for Microparticle Formulations PP-4, PP-6, Q, PP-5, PP-8, V, and Q, each containing 7525 HIGH IV polymer.

Within groups of microparticles containing a particular polymer type, levels of risedronate and profile shapes were similar regardless of the microparticle sizes. There appeared to be a trend toward slightly higher cumulative AUC during the first week after administration of microparticle batches having smaller particle sizes (e.g., DV₅₀ of about 30 microns).

### EXAMPLE 17

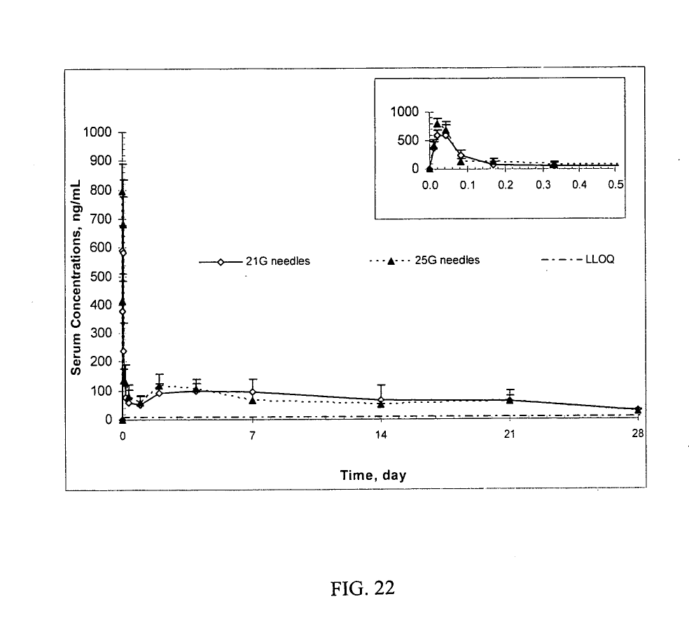

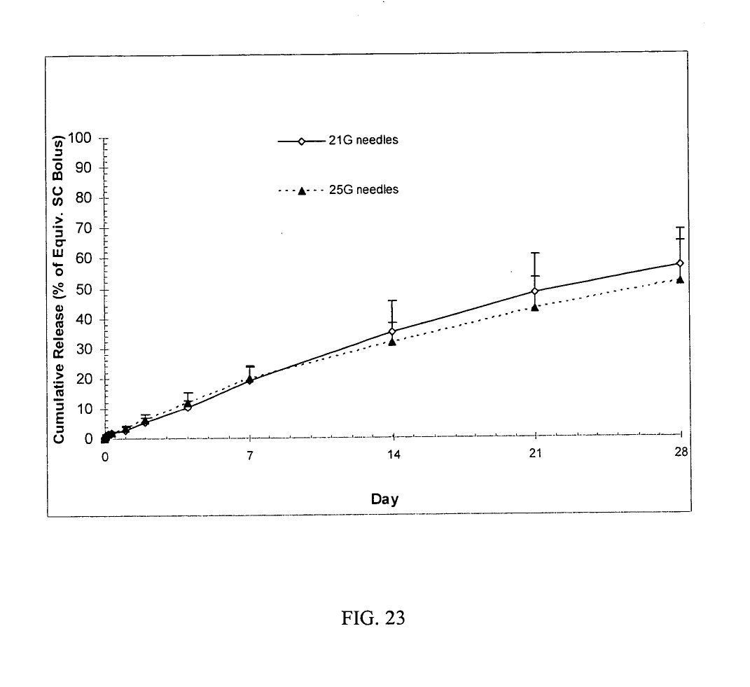

This example describes a study of the effect of needle gauge on PK profiles. Microparticles were made using Formulation PP-8 as described in Example 15. The microparticles were then injected into rats using 21 gauge and 25 gauge needles. In all cases, the microparticles were easily aspirated and injected with no abnormalities noted. As shown in FIGS. 22 and 23, there was no measurable impact on rat PK during four weeks following dose administration. For each animal treatment group, n=6. Each test subject received a normalized dose of microparticles having about 10 milligrams risedronate sodium per kilogram of body mass. In each case, the administered dose was normalized based on the measured risedronate sodium load in the microparticles as determined by nitrogen analysis, described supra.

### EXAMPLE 18

The following example describes the formation of microparticles that include alendronate monosodium and a biocompatible polymer using a water-in-oil-in-water (W/O/W) emulsion process.

Either 1.25 mL or 2.5 mL of reverse osmosis deionized (RODI) water was added to a 20 mL glass scintillation vial containing 301 mg of alendronate sodium bulk drug substance (BDS) (Sodium Nendronate BP, SaiQuest, San Diego, Calif.). The vial was then placed in an 80° C. water bath. The BDS was dissolved by swirling while the temperature was maintained at 80° C., thereby forming an aqueous solution of alendronate at a concentration of either about 200 or about 100 mg risedronate sodium/mL solution.

In some instances, the 1.25 or 2.5 mL of RODI water was mixed with a surfactant (i.e., polyvinyl alcohol (PVA), Pluronic F68 or TWEEN® 20) to form a 0.02%, 0.1%, or 0.5% (w/v) surfactant solution. The surfactant solution was then added the 20 mL glass scintillation vial containing 30 mg of alendronate sodium BDS as described above. 4.75 grams (g) of 100 4M polymer were mixed with 21.6 g of ethyl acetate, thereby forming a polymer/ethyl acetate combination with about 18 weight percent polymer. The polymer/ethyl acetate combination was poured into a stainless steel funnel with a valve on the bottom. A sonication microtip probe (Model No. CV17; Sonics and Materials, Inc., Danbury, Conn.) was placed about 1 cm below the surface of the polymer/ethyl acetate combination. 2.5 mL of the 80° C. aqueous solution of alendronate was drawn into a sterile 3 mL syringe with a 1.5 in (about 3.8 cm), 18 gauge needle. The sonication was turned on at 40% amplitude. The 80° C. aqueous solution of risedronate was then injected near the microtip probe over an injection time of 14 sec+/−5 sec. Sonication of the resulting mixture was continued for about 1 minute after the end of injection. Thus, a primary, or inner, emulsion (W/O) was formed.

A 0.25 in (about 0.64 cm) outside diameter, 34 element, static mixer constructed of 316 stainless steel (Model No. 04669-60; Cole-Parmer Instrument Co., Vernon Hills, Ill.) was primed for about 5 seconds by pumping a polyvinyl alcohol (PVA) solution, containing 1 weight percent PVA with 6.5 weight percent ethyl acetate, through a T-junction and into the mixer at about 700 to 1000 mL/min. Following priming, the flow rate of the PVA solution was maintained at about 700 to 1000 mL/min. The primary emulsion was then pumped through the other branch of the T-junction at a flow rate of 70 to 100 mL/min to combine with the PVA solution stream. The combined streams were directed into the static mixer. The primary emulsion was shunted into the PVA solution for about 30+/−5 seconds. Five seconds after the addition of the primary emulsion, the PVA solution stream was turned off.

The static mixer outlet was joined to a dip-tube which emptied into a quench tank. As the mixture of the primary emulsion and PVA solution left the static mixer, the combined stream flowed into the quench tank. The quench tank was initially charged with 3.5 liters of RODI water at room temperature. The quench tank was equipped with an impeller stirring at about 300 to about 400 rpm. Following addition of the combined primary emulsion and PVA solution streams, the quench tank was stirred for 30 minutes.

After 30 minutes of residence in the quench tank, a valve on the bottom of the quench tank was opened and the contents of the tank were directed into a 25 micron stainless steel sieve to collect the microparticles. The microparticles were washed in the sieve with a continuous flow of RODI water for about 3 to 5 minutes. The microparticles were transferred to a sterile glass dish and the dish was covered with a Kimwipe. The glass dish was placed in a freezer at −80° C. for at least about 30 minutes. The glass dish was then placed in a pre-chilled lyophilizer (Model No. TD-2C-MP; FTS Systems, Inc., Stone Ridge, N.Y.) with a shelf temperature of about −40° C. The following lyophilization program was then performed: started at −40° C., 150 millitorr (mT); ramped 2.5° C./min to −10° C.; held 5 hours at 300 mT; ramped 2.5° C./min to 30° C.; and held for 2 days at 300 mT. The microparticles were then poured into a sterilized 150 micron stainless steel sieve (6 cm diameter) and the microparticles were sieved by banging and breaking microparticle masses with a spatula. The fraction of the microparticles that passed through the sieve were collected as the microparticle product.

Table 24 shows conditions used to produce several batches of microparticles containing alendronate.

TABLE 24
Microparticle Formulations

| | Target | | | Alendronate |
|---|---|---|---|---|
| | Alendronate | Water in | Surfactant | Concentration |
| | Sodium | Aqueous | in Aqueous | in Aqueous |
| Formu- | Loading | Solution | Solution | Solution |
| lation | (wt %) | (mL) | (w/v) | (mg/ml) |
| SS | 5 | 2.5 | none | 100 |
| SS-1 | 5 | 2.5 | none | 100 |
| SS-2 | 5 | 2.5 | 0.02% TWEEN 20 | 100 |
| SS-3 | 5 | 2.5 | 0.1% TWEEN 20 | 100 |
| SS-4 | 5 | 2.5 | 0.5% TWEEN 20 | 100 |
| TT | 10 | 2.5 | none | 100 |
| TT-1 | 10 | 2.5 | none | 100 |
| TT-2 | 10 | 2.5 | 0.1% TWEEN 20 | 100 |
| TT-3 | 5 | 1.25 | none | 200 |
| TT-4 | 5 | 1.25 | none | 200 |
| TT-5 | 5 | 1.25 | none | 200 |
| TT-6 | 5 | 1.25 | 0.02% TWEEN 20 | 200 |
| TT-7 | 5 | 1.25 | 0.1% TWEEN 20 | 200 |
| TT-8 | 5 | 1.25 | 0.5% TWEEN 20 | 200 |
| UU-1 | 5 | 1.25 | none | 200 |
| UU-2 | 5 | 1.25 | none | 200 |
| UU-3 | 5 | 1.25 | 0.1% PVA | 200 |
| UU-4 | 5 | 1.25 | 0.1% Pluronic F68 | 200 |
| VV-1 | 5 | 2.5 | none | 100 |
| VV-2 | 5 | 2.5 | none | 100 |
| VV-3 | 5 | 2.5 | none | 100 |

Formulations VV-2 and VV-3 were made using a 1/8 inch ISG (Ross Engineering, Savannah, Ga.) and a 1/16 inch ISG (Ross Engineering), respectively. For Formulation VV-2, the flow rate of the PVA solution was about 1300 mL/min and the flow rate of the primary emulsion was about 130 mL/min to combine with the PVA solution stream. The combined streams were directed into the ISG static mixer. For Formulation VV-3, the flow rate of the PVA solution was about 800 mL/min and the flow rate of the primary emulsion was about 80 mL/min to combine with the PVA solution stream. The combined streams were directed into the ISG static mixer.

Table 25 shows the effect of microparticle particle size and surfactant on initial in vitro release of alendronate from the microparticles.

TABLE 25
Microparticle Characteristics

| | | | Alendronate | Alendronate | 24 hour In |
|---|---|---|---|---|---|
| | Microparticle | Presence | Loading | Incorporation | Vitro Release |
| | Size, DV₅₀ | of | (wt %, by | Efficiency | (Cumulative |
| Formulation | (microns) | Surfactant | N₂ analysis) | (%) | %) |
| SS | 78 | No | 5.1 | 102 | 9 |
| SS-1 | 60 | No | 5.4 | 108 | 23 |
| SS-2 | 60 | Yes | 5.3 | 106 | 14 |
| SS-3 | 61 | Yes | 5.3 | 107 | 13 |
| SS-4 | 59 | Yes | 5.3 | 106 | 5 |
| TT | 82 | No | 7.2 | 72 | 10 |
| TT-1 | 59 | No | 7.0 | 70 | 29 |
| TT-2 | 62 | Yes | 8.6 | 86 | 22 |

Table 26 shows the effect of alendronate concentration in the aqueous solution and surfactant on alendronate loading and on initial in vitro release of alendronate from the microparticles.

TABLE 26
Microparticle Characteristics

| | Alendronate | | | | |
|---|---|---|---|---|---|
| | Concentration | | | | 24 hour In |
| | in Aqueous | Presence | Microparticle | Alendronate | Vitro Release |
| | Solution | of | Size, DV₅₀ | Loading | (Cumulative |
| Formulation | (mg/ml) | Surfactant | (microns) | (wt %) | %) |
| SS | 100 | No | 78 | 5.1 | 9 |
| SS-1 | 100 | No | 60 | 5.4 | 23 |
| SS-2 | 100 | Yes | 60 | 5.3 | 14 |
| SS-3 | 100 | Yes | 61 | 5.3 | 13 |
| SS-4 | 100 | Yes | 59 | 5.3 | 5 |
| TT-3 | 200 | No | 72 | 3.02 | 11 |
| TT-4 | 200 | No | 56 | 2.4 | 2 |
| TT-5 | 200 | No | 54 | 2.6 | 11 |
| TT-6 | 200 | Yes | 56 | 3.2 | 12 |
| TT-7 | 200 | Yes | 56 | 3.1 | 11 |
| TT-8 | 200 | Yes | 55 | 4.5 | 9 |

Table 27 shows the effect of surfactant type in the aqueous solution (0.1% w/v) on alendronate loading and on initial in vitro release of alendronate from the microparticles.

TABLE 27
Microparticle Characteristics

| | | Alendronate | 24 hour In Vitro Release |
|---|---|---|---|
| Formulation | Surfactant | Loading (wt %) | (Cumulative %) |
| UU-1 | none | 2.6 | 11 |
| UU-2 | none | 2.4 | 2 |
| TT-7 | TWEEN 20 | 3.1 | 11 |
| UU-3 | PVA | 3.5 | 10 |
| UU-4 | Pluronic F68 | 3.4 | 11 |

Table 28 shows the effect of static mixer configuration on alendronate loading and on initial in vitro release of alendronate from the microparticles (DV₅₀ of approximately 60 microns).

TABLE 28
Microparticle Characteristics

| | | Alendronate | Alendronate | 24 hour In Vitro |
|---|---|---|---|---|
| Formu- | | Loading | Incorporation | Release |
| lation | Static Mixer | (wt %) | Efficiency (%) | (Cumulative %) |
| VV-1 | ¼ inch | 5.4 | 108 | 24 |
| | Cole-Parmer | | | |
| VV-2 | ⅛ inch ISG | 5.0 | 100 | 20 |
| VV-3 | 1/16 inch | 5.5 | 109 | 15 |
| | ISG | | | |

The results indicated that batches with surfactant had an improved load. Formulation VV-3, made using a 1/16 inch ISG static mixer, produced the lowest initial burst of alendronate.

### EXAMPLE 19

The following example describes a study of terminal sterilization of microparticles containing risedronate sodium by gamma-irradiation. Gamma-irradiation of microparticles could eliminate the need for aseptic validation, eliminate batch rejection due to an aseptic process breech, provide additional assurance of final product sterility, provide additional assurance of final product sterility, and could enable parametric lot release of commercial product without sterility testing.

Microparticles were produced using a method similar to that used to produce Formulation Q-5, described supra. Microparticles were vialed either at approximately 130 mg/vial or 1 to 2 grams/vial. Microparticle vials were then exposed to either 16 KiloGrays (kGy) or 26 kGy of Cobalt 60 gamma radiation at Steris, Inc. (Morton Grove, Ill.) for either 105 minutes or 159 minutes, respectively. Control vials (not exposed to radiation) were also prepared. The vials were then stored in at either 5° C. or 25° C. for three months.

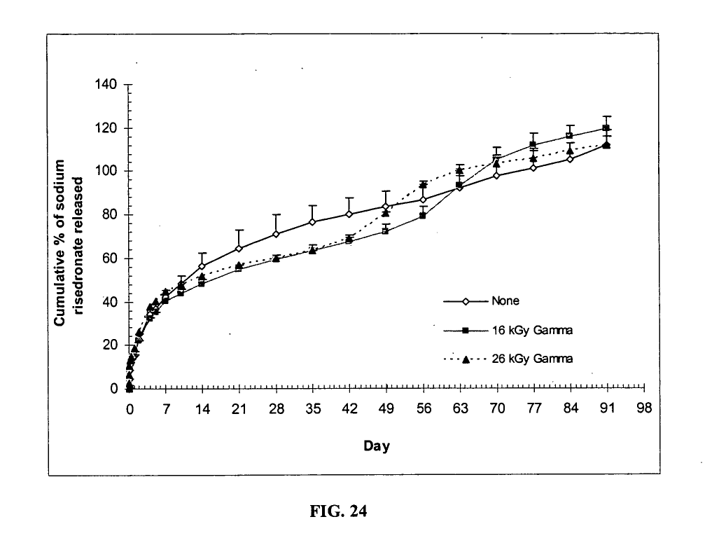

Samples of each of the microparticles before storage were set aside prior to storage for an in vivo study in rat. Rats (n=4) were injected with a normalized dose of microparticles (20 mg/kg). FIG. 24 shows a plot of mean (n=4) cumulative AUC, as a percentage of equivalent subcutaneous bolus injection, versus time (in days) post subcutaneous administration of gamma-irradiated and non-irradiated (control) microparticles, Formulation Q-5, described above.

Storage stability results through three months showed no significant impact of gamma-irradiation on the microparticles with respect to aspect (e.g., appearance, suspension, aspiration, and injection); incorporated risedronate sodium content and integrity; and microparticle size. There was detected a post-irradiation decrease in molecular weight that was dependent on the radiation dose. There was no significant impact on microparticle stability through 3 months. The data and observations suggest that sterilization by gamma-irradiation is feasible for microparticles which include a biocompatible polymer and a bisphosphonate such as risedronate sodium.

While this invention has been particularly shown and described with references to preferred embodiments thereof, it will be understood by those skilled in the art that various changes in form and details may be made therein without departing from the scope of the invention encompassed by the appended claims.

1. A method of forming microparticles that include a bisphosphonate and a polymer, comprising the steps of:
a) forming a water-in-oil emulsion by mixing an aqueous solution of the bisphosphonate with a combination of a poly(lactide) or a poly(lactide-co-glycolide) polymer and a polymer solvent, wherein the molar ratio of the lactide component to the glycolide component in the polymer is at least about 65:35; and
b) mixing at least one aqueous liquid with the water-in-oil emulsion to form a water-in-oil-in-water emulsion and to extract the polymer solvent from the polymer, thereby forming the microparticles.

2. The method of claim 1 wherein the molar ratio of the lactide component to the glycolide component of the polymer is about 65:35 to about 85:15.

3. The method of claim 1 wherein the inherent viscosity of the polymer measured in chloroform at 25° C. is no more than about 0.65 deciliters/gram (dL/g).

4. The method of claim 1 wherein the inherent viscosity of the polymer measured in chloroform at 25° C. is about 0.8 to about 0.85 deciliters/gram (dL/g).

5. The method of claim 1 wherein the poly(lactide) or the poly(lactide-co-glycolide) polymer includes an ester end group.

6. The method of claim 5 wherein the ester end group is selected from the group consisting of a methyl ester and a lauryl ester.

7. The method of claim 1 wherein the poly(lactide) or the poly(lactide-co-glycolide) polymer includes an acid end group.

8. The method of claim 7 wherein the acid end group is a free carboxyl end group.

9. The method of claim 1 wherein the bisphosphonate is a compound represented by the following chemical structure: 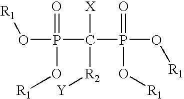
wherein,
R₁ is, independently, H, alkyl, aryl or heteroaryl;
X is H, —OR, or halogen;
R₂ is H, O, S, N, (CH₂)ₙ, branched alkylene, branched or straight alkenylene or alkynylene;
n is an integer from about 0 to about 18;
Y is H, R₁, halogen, amino, cyano or amido group;
or a pharmaceutically acceptable salt thereof.

10. The method of claim 9 wherein the bisphosphonate is selected from the group consisting of alendronate, risedronate, pamidronate, etidronate, tiludronate, ibandronate, pharmaceutically acceptable salts thereof and combinations thereof.

11. The method of claim 9 wherein the bisphosphonate is a compound represented by the following chemical structure: 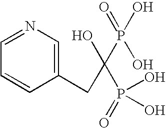
or a pharmaceutically acceptable salt thereof.

12. The method of claim 11 wherein the bisphosphonate is (1-hydroxy-2-(-3-pyridinyl)ethylidene)bis(phosphonic acid) monosodium salt.

13. The method of claim 1 wherein forming the water-in-oil emulsion includes mixing the aqueous solution with the combination of the polymer and the polymer solvent using rotor-stator mixing.

14. The method of claim 1 wherein forming the water-in-oil emulsion includes mixing the aqueous solution with the combination of the polymer and the polymer solvent using sonication.

15. The method of claim 1 wherein forming the water-in-oil emulsion includes mixing the aqueous solution with the combination of the polymer and the solvent using a high pressure homogenizer.

16. The method of claim 1 wherein the step of mixing at least one aqueous liquid with the water-in-oil emulsion to form a water-in-oil-in-water emulsion includes mixing the water-in-oil emulsion with an aqueous liquid in a static mixer.

17. The method of claim 16 wherein the water-in-oil emulsion is mixed in the static mixer at a water-in-oil emulsion flow rate of about 20 mL/min to about 1500 mL/min.

18. The method of claim 1 wherein the step of mixing at least one aqueous liquid with the water-in-oil emulsion to form a water-in-oil-in-water emulsion includes mixing the water-in-oil emulsion with an aqueous liquid that includes a surfactant.

19. The method of claim 18 wherein the surfactant is selected from the group consisting of polyvinyl alcohol, poloxamers, and polysorbates.

20. The method of claim 1 wherein the step of mixing at least one aqueous liquid with the water-in-oil emulsion includes mixing an aqueous liquid with the water-in-oil emulsion to form a water-in-oil-in-water emulsion and then mixing the water-in-oil-in-water emulsion with an aqueous liquid extraction medium.

21. The method of claim 20 wherein the aqueous liquid extraction medium is water.

22. The method of claim 1 further comprising the step of isolating the microparticles.

23. The method of claim 22 wherein isolating the microparticles includes filtering the microparticles from the at least one aqueous liquid.

24. The method of claim 22 wherein isolating the microparticles includes lyophilizing the microparticles.

25. A method of forming microparticles that include a bisphosphonate and a polymer, comprising the steps of:
a) forming a water-in-oil emulsion by mixing an aqueous solution of the bisphosphonate with a combination of a biocompatible polymer and a polymer solvent, wherein the concentration of the bisphosphonate in the aqueous solution is greater than the room temperature solubility limit of the bisphosphonate; and
b) mixing at least one aqueous liquid with the water-in-oil emulsion to form a water-in-oil-in-water emulsion and to extract the polymer solvent from the polymer, thereby forming the microparticles.

26. The method of claim 25 wherein the concentration of the bisphosphonate in the aqueous solution is at least about 75 mg/mL.

27. The method of claim 26 wherein the concentration of the bisphosphonate in the aqueous solution is at least about 100 mg/mL.

28. The method of claim 25 wherein the concentration of the bisphosphonate in the aqueous solution is at least about twice the room temperature solubility limit of the bisphosphonate.

29. The method of claim 26 wherein the aqueous solution of the bisphosphonate is prepared by heating a mixture of the bisphosphonate and water.

30. The method of claim 26 wherein the temperature of the aqueous solution is higher than room temperature.

31. The method of claim 30 wherein the temperature of the aqueous solution is at least about 50° C.

32. The method of claim 31 wherein the temperature of the aqueous solution is at least about 75° C.

33. The method of claim 30 wherein the temperature of the aqueous solution is about 75° C. to about 85° C.

34. The method of claim 30 wherein the concentration of the bisphosphonate in the aqueous solution is less than the solubility limit of the bisphosphonate at the temperature of the aqueous solution.

35. The method of claim 25 wherein the aqueous solution is a supersaturated solution of the bisphosphonate.

36. The method of claim 25 wherein the temperature of the aqueous solution is higher than the temperature of the combination of polymer and polymer solvent.

37. The method of claim 25 wherein the temperature of the combination of polymer and polymer solvent is about room temperature.

38. A method of forming microparticles that include a bisphosphonate and a polymer, comprising the steps of:
a) preparing an aqueous mixture of the bisphosphonate and a surfactant;
b) forming a water-in-oil emulsion by mixing the aqueous mixture with a combination of a biocompatible polymer and a polymer solvent;
c) forming a water-in-oil-in-water emulsion by mixing the water-in-oil emulsion with an aqueous liquid; and
d) removing the polymer solvent from the polymer, thereby forming the microparticles.

39. The method of claim 38 wherein the surfactant is selected from the group consisting of polyvinyl alcohol, poloxamers and polysorbates.

40. The method of claim 39 wherein the surfactant is poloxamer 188.

41. The method of claim 39 wherein the surfactant is polysorbate 20.

42. The method of claim 38 wherein the concentration of the bisphosphonate in the aqueous mixture is greater than the room temperature solubility limit of the bisphosphonate.

43. The method of claim 38 wherein the temperature of the aqueous mixture is higher than room temperature.

44. A method of forming microparticles that include a bisphosphonate and a polymer, comprising the steps of:
a) forming a water-in-oil emulsion by mixing an aqueous solution consisting essentially of water and the bisphosphonate with a combination of a biocompatible polymer and a polymer solvent; and
b) mixing at least one aqueous liquid with the water-in-oil emulsion to form a water-in-oil-in-water emulsion and to extract the polymer solvent from the polymer, thereby forming the microparticles.

45. A method of forming microparticles that include a bisphosphonate and a polymer, comprising the steps of:
a) forming a bisphosphonate suspension in a combination consisting essentially of a biocompatible polymer and a polymer solvent; and
b) mixing at least one aqueous liquid with the bisphosphonate suspension to form a solid-in-oil-in-water emulsion and to extract the polymer solvent from the polymer, thereby forming the microparticles.

46. A method of forming microparticles that include a bisphosphonate and a polymer, comprising the steps of:
a) forming a water-in-oil emulsion by mixing an aqueous solution of the bisphosphonate with a combination of a biocompatible polymer and a polymer solvent; and
b) mixing at least one aqueous liquid with the water-in-oil emulsion to form a water-in-oil-in-water emulsion and to extract the polymer solvent from the polymer, thereby forming the microparticles.

47. The method of claim 46 wherein the step of mixing at least one aqueous liquid with the water-in-oil emulsion includes mixing the water-in-oil emulsion with an aqueous liquid in a static mixer.

48. The method of claim 47 wherein the water-in-oil emulsion is mixed in the static mixer at a water-in-oil emulsion flow rate of about 20 mL/min to about 1500 mL/min.

49. The method of claim 46 wherein the step of mixing at least one aqueous liquid with the water-in-oil emulsion includes mixing the water-in-oil emulsion with an aqueous liquid that includes a surfactant.

50. The method of claim 49 wherein the surfactant includes polyvinyl alcohol.

51. The method of claim 46 wherein the step of mixing at least one aqueous liquid with the water-in-oil emulsion includes mixing an aqueous liquid extraction medium with the water-in-oil-in-water emulsion.

52. The method of claim 51 wherein the aqueous liquid extraction medium is water.

53. The method of claim 51 wherein the aqueous liquid extraction medium has a room temperature capacity for the polymer solvent of at least about 5 weight percent.

54. The method of claim 53 wherein the aqueous liquid extraction medium has a room temperature capacity for the polymer solvent of at least about 7 weight percent.

55. The method of claim 46 wherein the polymer solvent is represented by the chemical structure, R₃COOR₄, wherein R₃ and R₄ are, independently, alkyl groups having from about 1 to about 4 carbon atoms.

56. The method of claim 55 wherein the polymer solvent is ethyl acetate.

57. A method of forming microparticles that include a bisphosphonate and a polymer, comprising the steps of:
a) forming a water-in-oil emulsion by mixing an aqueous solution of the bisphosphonate with a combination of a biocompatible polymer and a polymer solvent, wherein the concentration of the bisphosphonate in the aqueous solution is greater than the room temperature solubility limit of the bisphosphonate;
b) forming a water-in-oil-in-water emulsion by mixing a first aqueous liquid with the water-in-oil emulsion; and
c) extracting the polymer solvent from the polymer into a second aqueous liquid, thereby forming the microparticles.

58. The method of claim 57 wherein the bisphosphonate is (1-hydroxy-2-(-3-pyridinyl)ethylidene)bis(phosphonic acid) monosodium salt.

59. The method of claim 57 wherein the concentration of the bisphosphonate in the aqueous solution is at least about 100 mg/mL.

60. The method of claim 57 wherein the temperature of the aqueous solution is about 75° C. to about 85° C.

61. The method of claim 57 wherein the temperature of the combination of the biocompatible polymer and the polymer solvent is about room temperature.

62. The method of claim 57 wherein the biocompatible polymer is a poly(lactide) or a poly(lactide-co-glycolide).

63. The method of claim 62 wherein the molar ratio of the lactide component to the glycolide component in the biocompatible polymer is about 65:35 to about 100:0.

64. The method of claim 57 wherein the polymer solvent is ethyl acetate.

65. The method of claim 57 wherein the aqueous liquid contains a surfactant.

66. The method of claim 65 wherein the surfactant is selected from the group consisting of polyvinyl alcohol, poloxamers and polysorbates.

67. The method of claim 66 wherein the surfactant is polyvinyl alcohol.

68. The method of claim 57 wherein forming the water-in-oil-in-water emulsion includes mixing the water-in-oil emulsion with the first aqueous liquid in a static mixer.

69. The method of claim 57 wherein the second aqueous liquid is water.

70. The method of claim 57 further comprising the step of isolating the microparticles.

71. The method of claim 70 wherein isolating the microparticles includes filtering the microparticles from the first and second aqueous liquids.

72. The method of claim 70 wherein isolating the microparticles includes lyophilizing the microparticles.

73. Microparticles prepared by the method of claim 57.

74. A pharmaceutical composition for the sustained release of a bisphosphonate, comprising the microparticles prepared by the method of claim 57.

75. A method for treating a patient in need of therapy, comprising the step of administering to the patient a therapeutically effective amount of the microparticles made by the method of claim 57.

76. The method of claim 75 wherein administering the microparticles to the patient includes intramuscular injection of the microparticles.

77. The method of claim 75 wherein administering the microparticles to the patient includes subcutaneous injection of the microparticles.

78. Microparticles consisting essentially of a biocompatible polymer and at least about 3 weight percent of risedronate or a salt thereof.

79. The microparticles of claim 78 wherein the biocompatible polymer is a poly(lactide) or a poly(lactide-co-glycolide).

80. The microparticles of claim 79 wherein the molar ratio of the lactide component to the glycolide component in the biocompatible polymer is about 65:35 to about 100:0.

81. The microparticles of claim 78 wherein the microparticles have been gamma-irradiated.

82. The microparticles of claim 81 wherein the microparticles have been gamma-irradiated with about 15 to about 45 kGy of gamma radiation.

83. The microparticles of claim 82 wherein the microparticles have been gamma-irradiated with about 16 kGy of gamma radiation.

84. The microparticles of claim 82 wherein the microparticles have been gamma-irradiated with about 26 kGy of gamma radiation.

85. The microparticles of claim 78 wherein the microparticles have an in vitro 24-hour cumulative risedronate release of less than about 10 weight percent from the microparticles.

86. The microparticles of claim 85 wherein the in vitro 24-hour cumulative risedronate release is in a phosphate buffered saline composition at 37° C.

87. The microparticles of claim 78 wherein the microparticles, upon administration to a patient, have an in vivo duration of risedronate release from the microparticles of at least about 60 days.

88. Microparticles consisting essentially of a bisphosphonate and a biocompatible polymer wherein the microparticles have an in vitro 24-hour cumulative bisphosphonate release of less than about 15 weight percent.

89. The microparticles of claim 88 having an in vitro 24-hour cumulative bisphosphonate release of less than about 10 weight percent.

90. The microparticles of claim 89 having an in vitro 24-hour cumulative bisphosphonate release of less than about 5 weight percent.

91. The microparticles of claim 88 wherein the in vitro 24-hour bisphosphonate release is in a phosphate buffered saline composition at 37° C. containing 0.02 weight percent polysorbate 20.

92. The microparticles of claim 88 wherein the bisphosphonate is selected from the group consisting of alendronate, risedronate, pamidronate, etidronate, tiludronate, ibandronate, pharmaceutically acceptable salts thereof and combinations thereof.

93. The microparticles of claim 88 wherein the bisphosphonate is (1-hydroxy-2-(-3-pyridinyl)ethylidene)bis(phosphonic acid) monosodium salt.

94. The microparticles of claim 88 wherein the biocompatible polymer is a poly(lactide) or a poly(lactide-co-glycolide).

95. The microparticles of claim 88 wherein the molar ratio of the lactide component to the glycolide component in the biocompatible polymer is about 65:35 to about 100:0.

96. The microparticles of claim 88 wherein the microparticles, upon administration to a patient, have an in vivo duration of bisphosphonate release from the microparticles of at least about 30 days.

97. The microparticles of claim 96 wherein the microparticles, upon administration to a patient, have an in vivo duration of bisphosphonate release from the microparticles of at least about 60 days.

98. Microparticles consisting essentially of a bisphosphonate and a biocompatible polymer wherein the microparticles cause a local site reaction in vivo upon parenteral administration to a patient that is substantially similar to a local site reaction caused by placebo microparticles that include the biocompatible polymer.

99. Microparticles consisting essentially of a bisphosphonate and a biocompatible polymer wherein the microparticles have clinically acceptable local tolerability in vivo upon administration to a patient.

100. The microparticles of claim 99 wherein the microparticles cause a local site reaction in vivo upon parenteral administration to a patient that is substantially similar to a local site reaction caused by placebo microparticles that include the biocompatible polymer.

101. The microparticles of claim 99 wherein the microparticles cause a local site reaction in vivo upon parenteral administration to a patient that is substantially reduced as compared to a local site reaction caused by a parenteral administration to the patient of a bisphosphonate not formed into microparticles with a biocompatible polymer.

102. Microparticles comprising:
a) a poly(d,l-lactide-co-gylcolide) polymer having about 75 mol % d,l-lactide, about 25 mol % glycolide, and a lauryl ester end group; and
b) risedronate or a salt thereof;
wherein the volume median diameter of the microparticles is about 20 to about 60 microns.

103. The microparticles of claim 102 wherein the volume median diameter of the microparticles is about 45 to about 55 microns

104. The microparticles of claim 102 wherein the volume median diameter of the microparticles is about 35 to about 45 microns.

105. The microparticles of claim 102 wherein the volume median diameter of the microparticles is about 25 to about 35 microns.

106. The microparticles of claim 102 wherein the polymer has an inherent viscosity measured in chloroform at 25° C. of about 0.8 to about 0.9 dL/g.

107. The microparticles of claim 102 wherein the risedronate or the salt thereof is present in the microparticles at a concentration of about 3 to about 6 percent by weight.

108. Microparticles comprising:
a) a poly(d,l-lactide-co-gylcolide) polymer having about 65 mol % d,l-lactide, about 35 mol % glycolide, and a lauryl ester end group; and
b) risedronate or a salt thereof;
wherein the volume median diameter of the microparticles is about 40 to about 60 microns.

109. The microparticles of claim 108 wherein the volume median diameter of the microparticles is about 45 to about 55 microns.

110. The microparticles of claim 108 wherein the polymer has an inherent viscosity measured in chloroform at 25° C. of about 0.5 to about 0.65 dL/g.

111. The microparticles of claim 108 wherein the risedronate or the salt thereof is present in the microparticles at a concentration of about 3 to about 6 percent by weight.

112. Microparticles comprising:
a) a poly(d,l-lactide) polymer having a methyl ester end group; and
b) risedronate or a salt thereof;
wherein the volume median diameter of the microparticles is about 40 to about 60 microns.

113. The microparticles of claim 112 wherein the volume median diameter of the microparticles is about 45 to about 55 microns.

114. The microparticles of claim 112 wherein the polymer has an inherent viscosity measured in chloroform at 25° C. of about 0.48 dL/g.

115. The microparticles of claim 112 wherein the risedronate or the salt thereof is present in the microparticles at a concentration of about 3 to about 6 percent by weight.

116. A method for treating a patient in need of therapy, comprising:
administering to the patient a therapeutically effective amount of microparticles consisting essentially of a biocompatible polymer and risedronate or a salt thereof; wherein the microparticles have an in vitro 24-hour cumulative risedronate release from the microparticles of less than about 15 weight percent.

117. The method of claim 116 wherein the in vitro 24-hour cumulative risedronate release from the microparticles is less than about 10 weight percent.

118. The method of claim 116 wherein the in vitro 24-hour cumulative risedronate release is in a phosphate buffered saline composition at 37° C.

119. A method for treating a patient in need of therapy, comprising:
administering to the patient a therapeutically effective amount of microparticles consisting essentially of a biocompatible polymer and risedronate or a salt thereof; wherein the microparticles have an in vivo duration of risedronate release from the microparticles of at least about 60 days.
